(19) United States
(12) Patent Application Publication
(10) Pub. No.: US 2023/0332249 A1
(43) Pub. Date: Oct. 19, 2023

(54) IDENTIFYING THE PRESENCE OF METASTATIC CANCER AND TISSUE OF ORIGIN WITH MICROBIAL NUCLEIC ACIDS

(71) Applicant: THE REGENTS OF THE UNIVERSITY OF CALIFORNIA, Oakland, CA (US)

(72) Inventor: Gregory D. Poore, La Jolla, CA (US)

(21) Appl. No.: 18/044,541

(22) PCT Filed: Sep. 21, 2021
(86) PCT No.: PCT/US2021/051261

§ 371 Date: Mar. 8, 2023

Related U.S. Application Data
(60) Provisional application No. 63/105,624, filed on Oct. 26, 2020.
(60) Provisional application No. 63/081,075, filed on Sep. 21, 2020.

(51) Int. Cl. C12Q 1/689; C12Q 1/6886; G16H 50/20; G16B 30/00

(52) U.S. Cl. CPC C12Q 1/689; C12Q 1/6886; G16H 50/20; G16B 30/00; C12Q 2600/112

(57) ABSTRACT

Methods for the detection of metastatic cancer and determination of its tissue of origin on the basis of non-human, microbial nucleic acids in tissue or blood.

### CROSS REFERENCE TO RELATED APPLICATIONS

This application claims the priority benefit of U.S. Provisional Application Nos. 63/081,075 and 63/105,624, filed Sep. 21, 2020 and Oct. 26, 2020, respectively, which applications are incorporated herein by reference.

### GOVERNMENT SPONSORSHIP

This invention was made with government support under Grant No. F30 CA243480 awarded by the National Institutes of Health. The government has certain rights to in the invention.

### TECHNICAL FIELD

The present invention relates to identifying the presence of a metastatic cancer and/or its tissue of origin with non-human, microbial nucleic acids present in tissue and liquid biopsies. In at least one embodiment of the present invention, a machine-learning (ML) model is trained as a diagnostic model to discriminate between and within types of metastatic cancer.

### BACKGROUND

Increasing evidence indicates a key role for the bacterial, viral, fungal, archaeal, and phage microbiota in carcinogenesis. In fact, as much as 20% of the global cancer burden has been estimated to be directly caused by microbial agents. Many researchers believe the potential mechanism is through microbial influence on the immune system, with their abilities to dial up or dampen down inflammation as well as to manipulate the capabilities of the subject's immune cells among other mechanisms.

Based on data from studies using gnotobiotic mouse models colonized with one or more specific bacteria, it appears that microbiota can alter cancer susceptibility and progression by diverse mechanisms, such as modulating inflammation, inducing DNA damage, and producing metabolites involved in oncogenesis or tumor suppression. In addition to carcinogenesis and cancer progression, emerging evidence suggests that microbiota can predict response to cancer treatment or be manipulated for improving cancer treatment, including “traditional” chemotherapies (e.g. gemcitabine) and more “innovative” immunotherapies (e.g. PD-1 blockade).

While much of the literature has focused on examining compositions or functions of the host gut microbiome and its influence on cancer, recent examples in the literature have explored cancer-associated microbiota within primary tumor tissues or within the blood of patients bearing primary tumors (PMID: 32214244, 32467386, 29567829, 31578522). Primary tumor-associated microbiota have often been of research interest because of their potentially causal relationship with tumor formation and because of the ease of accessing single primary tumors in comparison to their multiple metastatic counterparts.

However, the majority of cancer deaths do not derive from primary tumors but rather from metastases, and very little remains known about the relationship between cancer-associated microbiota and metastatic cancers. If this gap in the field could be addressed, it could lead to new kinds of cancer diagnostics that prevent substantial patient morbidity and mortality through early detection of the presence and/or tissue of origin of metastatic cancers. Moreover, the accurate identification of the tissue of origin of a metastatic cancer is critical for guiding which clinical treatment should be given to a patient. As a contrived example, a metastatic lung cancer found in the brain of a patient will have different clinical management than a brain cancer that originated in the brain (i.e., a primary tumor) of a patient. Thus, methods that improve the tissue of origin diagnosis of a metastatic cancer also influence the optimal type or dosing of a given treatment and the prognosis of the patient.

Historically, the process of identifying the tissue of origin of a metastatic cancer has relied on obtaining human molecular information from a metastatic tissue biopsy: immunohistochemistry (IHC) protein staining, sequencing human DNA (e.g., to identify mutations known to be associated with a particular primary tumor type), sequencing modifications of DNA (e.g., the epigenome), or sequencing human RNA (e.g., to identify gene expression patterns associated with a particular primary tumor type). Yet, the accuracy of these methods to localize the tissue of origin of a metastatic tumor has been limited. For example, Weiss et al. (PMID: 23287002) reported an accuracy level of just 69% using IHC methods and only 79% when using a 92-gene expression signature on the same samples. These results imply a failure rate when identifying the tissue of origin for >20% of patients' metastatic cancers, which is striking given that the vast majority of all cancer deaths are due to metastases. These low accuracy rates are reflect how many metastatic tumors lose the original cellular markers of their primary tumor tissue, making their source difficult to identify confidently and quickly with human information, which can spur clinically invasive, expensive, and urgent hunts for patients' primary tumors.

For the current scientific state of the art regarding cancer-associated microbes, the following is known: (i) many cancer-associated microbes are located intracellularly inside primary tumor cancer cells and adjacent immune cells (PMID: 32467386), (ii) virtually all primary tumors harbor cancer type-specific microbiota (PMID: 32214244), and (iii) intracellular microbes may travel within the cancer cells as they metastasize from a primary tumor in the case of colon cancer (PMID: 29170280).

However, what is unknown and of critical importance is the following: (i) whether the microbiota of metastases faithfully reflect their tissue of origin or whether the new body site of the metastases (compared to the primary tumor) disrupts the microbial composition or function; (ii) whether all cancer types, particularly those excluding colon cancer, share intracellular (or extracellular) microbes between primary tumors and their metastases, which would affect the viability of a pan-cancer diagnostic approach for metastases that relies on microbial information; (iii) whether the microbiota of metastases can be detected in the blood, and, if so, if such information would be informative of the cancer's tissue of origin.

Previously, WO2020093040A1 focused on developing new cancer diagnostics for primary tumors using non-human, microbial nucleic acids in patient tissue and blood. Additionally, US20180291463A1, WO2018200813A1, and WO2018031545A1 describe a microarray-based technology for detecting pre-selected (“biased”) populations of microbes in primary tumor samples (NOT metastases and NOT blood or other bodily fluids). US20180223338 describes using the primary tumor tissue microbiome or saliva microbiome in identifying and diagnosing head and neck cancer. US20180258495A1 describes using the primary tumor tissue microbiome or fecal microbiome to detect colon cancer, some kinds of mutations associated with colon cancer, and a kit to collect and amplify the corresponding microbes.

### SUMMARY OF THE INVENTION

The disclosure of the present invention provides, according to at least one embodiment, a method to accurately diagnose or determine the presence or lack thereof metastatic cancer, its tissue of origin, and its likelihood to response to certain therapies solely using nucleic acids of non-human origin from a human tissue biopsy or blood-derived sample.

In embodiments, the invention provides a method for broadly creating patterns of microbial presence or abundance (‘signatures’) that are associated with the presence and/or type of metastatic cancer using blood-derived tissues. These signatures can then be deployed to diagnose the presence and/or tissue of origin of metastatic cancer in a human.

In embodiments, the invention provides a method for broadly creating patterns of microbial presence or abundance that are associated with the tissue of origin of metastatic cancers using metastatic tumor tissues. These signatures can then be deployed to diagnose the presence and/or tissue of origin of metastatic cancer in a human.

In embodiments, the invention provides a method for determining a presence or lack thereof metastatic cancer of a subject, comprising: detecting a microbial presence in a biological sample of a subject with cancer; removing contaminated microbial features from the microbial presence, thereby producing a decontaminated microbial presence; comparing the decontaminated microbial presence to a microbial presence of one or more biological samples from one or more subjects with cancer, thereby generating a microbial-cancer comparison dataset; and determining the presence or lack thereof metastatic cancer of the subject from the microbial cancer comparison dataset.

In embodiments, the invention provides a method for determining a presence or lack thereof metastatic cancer of a subject as described above/below, wherein determining the presence or lack thereof metastatic cancer of the subject from the microbial cancer comparison dataset comprises identifying a tissue of origin of the metastatic cancer.

In embodiments, the invention provides a method for determining a presence or lack thereof metastatic cancer of a subject as described above/below, wherein the microbial presence further comprises a microbial abundance. The microbial presence or abundance may, for example, comprises the following non-mammalian domains of life: bacteria, fungi, viruses, archaea, protozoa, bacteriophages, or any combination thereof.

In embodiments, the invention provides a method for determining a presence or lack thereof metastatic cancer of a subject as described above/below, wherein the microbial presence or abundance is measured by ecological shotgun sequencing, quantitative polymerase chain reaction, immunohistochemistry, in situ hybridization, flow cytometry, host whole genome sequencing, host transcriptomic sequencing, cancer whole genome sequencing, cancer transcriptomic sequencing, or any combination thereof.

In embodiments, the invention provides a method for determining a presence or lack thereof metastatic cancer of a subject as described above/below, wherein the microbial presence or abundance is measured by amplification of the following nucleic acid regions of microbial origin: V1, V2, V3, V4, V5, V6, V7, V8, V9 variable domain region of 16S rRNA, the internal transcribed spacer (ITS) region of the 18S rRNA, or any combination thereof.

In embodiments, the invention provides a method for determining a presence or lack thereof metastatic cancer of a subject as described above/below, wherein the microbial presence or abundance is detected by nucleic acid measurement that targets microbial DNA, RNA, or any combination thereof, wherein the nucleic acid measurement that targets microbial DNA, RNA, or any combination thereof, occurs simultaneously with a measurement of the subject's mammalian DNA, RNA, or any combination thereof.

In embodiments, the invention provides a method for determining a presence or lack thereof metastatic cancer of a subject as described above/below, wherein the metastatic cancer comprises: Acute Myeloid Leukemia, Adrenocortical Carcinoma, Bladder Urothelial Carcinoma, Brain Lower Grade Glioma, Breast Invasive Carcinoma, Cervical Squamous Cell Carcinoma and Endocervical Adenocarcinoma, Cholangiocarcinoma, Colon Adenocarcinoma, Lymphoid Neoplasm Diffuse Large B-cell Lymphoma, Esophageal Carcinoma, Glioblastoma Multiforme, Head and Neck Squamous Cell Carcinoma, Kidney Chromophobe, Kidney Renal Clear Cell Carcinoma, Kidney Renal Papillary Cell Carcinoma, Liver Hepatocellular Carcinoma, Lung Adenocarcinoma, Lung Squamous Cell Carcinoma, Mesothelioma, Ovarian Serous Cystadenocarcinoma, Pancreatic Adenocarcinoma, Pheochromocytoma and Paraganglioma, Prostate Adenocarcinoma, Rectum Adenocarcinoma, Sarcoma, Skin Cutaneous Melanoma, Stomach Adenocarcinoma, Testicular Germ Cell Tumors, Thyroid Carcinoma, Thymoma, Uterine Carcinosarcoma, Uterine Corpus Endometrial Carcinoma, Uveal Melanoma, or any combination thereof.

In embodiments, the invention provides a method for determining a presence or lack thereof metastatic cancer of a subject as described above/below, wherein the metastatic cancer comprises a cancer type, wherein the cancer type comprises: lung cancer, prostate cancer, melanoma cancer, breast cancer, thyroid cancer, or any combination thereof.

In embodiments, the invention provides a method for determining a presence or lack thereof metastatic cancer of a subject as described above/below, wherein the contaminated microbial features comprise taxonomic assignment of the microbial presence.

In embodiments, the invention provides a method for determining a presence or lack thereof metastatic cancer of a subject as described above/below, wherein removing contaminated microbial features is optional and not necessarily required.

In embodiments, the invention provides a method for determining a presence or lack thereof metastatic cancer of a subject as described above/below, wherein the biological samples of comparison used to form the microbial-cancer comparison dataset derive from subjects with one or more primary tumors, metastatic tumors, or any combination thereof.

In embodiments, the invention provides a method for determining a presence or lack thereof metastatic cancer of a subject as described above/below, wherein the microbial-cancer comparison dataset further comprises mammalian features, wherein the mammalian features comprise: immunohistochemistry protein markers of tumor tissue, tumor tissue DNA, tumor tissue RNA, tumor tissue methylation patterns, cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, methylation patterns of circulating tumor cell derived RNA, or any combination thereof.

In embodiments, the invention provides a method for determining a presence or lack thereof metastatic cancer of a subject as described above/below, wherein the biological sample comprises a tissue sample, liquid biopsy, whole blood biopsy, or any combination thereof. The biological sample may further comprise one or more constituents of whole blood comprising: plasma, white blood cells, red blood cells, platelets, or any combination thereof.

In embodiments, the invention provides a method of administering a treatment to treat metastatic cancer of a subject based on microbial presence, comprising: detecting a microbial presence in a biological sample from the subject with cancer; removing contaminated microbial features of the microbial presence, thereby producing a decontaminated microbial presence; generating an association between the decontaminated microbial presence and the metastatic cancer present in the subject; and administering to the subject the treatment determined by the association between the decontaminated microbial presence and the metastatic cancer.

In embodiments, the invention provides a method of administering a treatment to treat metastatic cancer of a subject based on microbial presence as described above/below, wherein the microbial presence further comprises a microbial abundance, wherein the microbial presence or abundance comprise the following non-mammalian domains of life: bacteria, fungi, viruses, archaea, protozoa, bacteriophages, or any combination thereof.

In embodiments, the invention provides a method of administering a treatment to treat metastatic cancer of a subject based on microbial presence as described above/below, wherein the contaminated microbial features comprise taxonomic assignment of the microbial presence.

In embodiments, the invention provides a method of administering a treatment to treat metastatic cancer of a subject based on microbial presence as described above/below, wherein removing contaminated microbial features of the microbial presence is an optional step and the association may be generated between the detected microbial presence and the metastatic cancer present in the subject.

In embodiments, the invention provides a method of administering a treatment to treat metastatic cancer of a subject based on microbial presence as described above/below, wherein the biological sample comprises a tissue sample, liquid biopsy, whole blood biopsy, or any combination thereof. The biological sample may further comprise one or more constituents of whole blood comprising: plasma, white blood cells, red blood cells, platelets, or any combination thereof.

In embodiments, the invention provides a method of administering a treatment to treat metastatic cancer of a subject based on microbial presence as described above/below, wherein the treatment is not metabolized or rendered inactive by the decontaminated microbial presence.

In embodiments, the invention provides a method of administering a treatment to treat metastatic cancer of a subject based on microbial presence as described above/below, wherein the treatment comprises: a small molecule, a hormone therapy, a biologic, an engineered host-derived cell type or types, a probiotic, an engineered bacterium, a natural-but-selective virus, an engineered virus, a bacteriophage, or any combination thereof.

In embodiments, the invention provides a method of administering a treatment to treat metastatic cancer of a subject based on microbial presence as described above/below, wherein the metastatic cancer comprises: Acute Myeloid Leukemia, Adrenocortical Carcinoma, Bladder Urothelial Carcinoma, Brain Lower Grade Glioma, Breast Invasive Carcinoma, Cervical Squamous Cell Carcinoma and Endocervical Adenocarcinoma, Cholangiocarcinoma, Colon Adenocarcinoma, Lymphoid Neoplasm Diffuse Large B-cell Lymphoma, Esophageal Carcinoma, Glioblastoma Multiforme, Head and Neck Squamous Cell Carcinoma, Kidney Chromophobe, Kidney Renal Clear Cell Carcinoma, Kidney Renal Papillary Cell Carcinoma, Liver Hepatocellular Carcinoma, Lung to Adenocarcinoma, Lung Squamous Cell Carcinoma, Mesothelioma, Ovarian Serous Cystadenocarcinoma, Pancreatic Adenocarcinoma, Pheochromocytoma and Paraganglioma, Prostate Adenocarcinoma, Rectum Adenocarcinoma, Sarcoma, Skin Cutaneous Melanoma, Stomach Adenocarcinoma, Testicular Germ Cell Tumors, Thyroid Carcinoma, Thymoma, Uterine Carcinosarcoma, Uterine Corpus Endometrial Carcinoma, Uveal Melanoma, or any combination thereof.

In embodiments, the invention provides a method of administering a treatment to treat metastatic cancer of a subject based on microbial presence as described above/below, wherein the treatment comprises an adjuvant given in combination with a primary treatment against the metastatic cancer to improve efficacy of the primary treatment. The adjuvant may, for example, be an antibiotic or an anti-microbial

In embodiments, the invention provides a method of administering a treatment to treat metastatic cancer of a subject based on microbial presence as described above/below, wherein, the treatment is based on microbial constituents or antigens associated with the metastatic cancer or the metastatic cancer's environment. The treatment may comprise an adoptive cell transfer to target microbial antigens, a cancer vaccine against microbial antigens, a monoclonal antibody against microbial antigens, an antibody-drug-conjugate designed to at least partially target microbial antigens, a multi-valent antibody, antibody fragment, antibody derivative thereof designed to at least partially target one or more microbial antigens, or any combination thereof.

In embodiments, the invention provides a method of administering a treatment to treat metastatic cancer of a subject based on microbial presence as described above/below, wherein the treatment comprises an antibiotic targeted against a class of functionally or biologically similar microbes of the microbial presence. The treatment may further comprise two or more treatment types, wherein the two or more treatment types are combined such that at least one type of the two or more treatment types exploits the microbial presence or abundance associated with the metastatic cancer or the metastatic cancer environment to enhance therapeutic efficacy.

In embodiments, the invention provides a method of administering a to treatment to treat metastatic cancer of a subject based on microbial presence as described above/below, wherein the association between the decontaminated microbial presence and the metastatic cancer further comprises the origin, type, or any combination thereof the metastatic cancer.

In embodiments, the invention provides a system configured to determine a presence or absence of metastatic cancer of a subject, comprising: one or more processors; and a non-transient computer readable storage medium including software, wherein the software comprises executable instructions that, as a result of execution, cause the one or more processors of the computer system to: obtain first data associated with one or more nucleic acid molecules of a biological sample from the subject with cancer; separate microbial nucleic acids from non-microbial nucleic acids of the first data associated with the one or more nucleic acids of the biological sample, thereby determining second data; identify, based on the second data, a microbial presence of the microbial nucleic acids; remove contaminated microbial features of the microbial presence from the second data, thereby producing a table of decontaminated microbial presence; input the table of decontaminated microbial presence into a machine-learning model; and receive from the machine-learning model, an output that indicates the presence or the absence of the metastatic cancer. In embodiments, the invention provides a system configured to determine a presence or absence of metastatic cancer of a subject, wherein the system comprises an Illumina NovaSeq 6000 instrument. The Illumina NovaSeq 6000 instrument may be communicatively coupled (e.g., via a network connection) to a network storage location that is accessible to one or more computer system that are able to access and process data generated by the Illumina NovaSeq 6000 instrument.

In embodiments, the invention provides a system configured to determine a presence or absence of metastatic cancer of a subject as described above/below, wherein the microbial presence further comprises a microbial abundance, wherein the microbial presence or abundance comprise the following non-mammalian domains of life: bacteria, fungi, viruses, archaea, protozoa, bacteriophages, or any combination thereof.

In embodiments, the invention provides a system configured to determine a presence or absence of metastatic cancer of a subject as described above/below, wherein the system further determines the tissue of origin of the metastatic cancer.

In embodiments, the invention provides a system configured to determine a presence or absence of metastatic cancer of a subject as described above/below, wherein the decontaminated microbial features comprise taxonomic assignment of the microbial presence.

In embodiments, the invention provides a system configured to determine a presence or absence of metastatic cancer of a subject as described above/below, wherein remove contaminated microbial features is optional.

In embodiments, the invention provides a system configured to determine a presence or absence of metastatic cancer of a subject as described above/below, wherein the microbial and non-microbial nucleic acids are separated by aligning the one or more nucleic acid molecules against a reference database of microbial and non-microbial genomes.

In embodiments, the invention provides a system configured to determine a presence or absence of metastatic cancer of a subject as described above/below, wherein the microbial and non-microbial nucleic acids are separated without aligning the one or more nucleic acid molecules against a reference genome database.

In embodiments, the invention provides a system configured to determine a presence or absence of metastatic cancer of a subject as described above/below, wherein the table of decontaminated microbial presence further comprise mammalian features, wherein the mammalian features comprise: immunohistochemistry protein markers of tumor tissue, tumor tissue DNA, tumor tissue RNA, tumor tissue methylation patterns, cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, methylation patterns of circulating tumor cell derived RNA, or any combination thereof.

In embodiments, the invention provides a system configured to determine a presence or absence of metastatic cancer of a subject as described above/below, wherein the metastatic cancer comprises: Acute Myeloid Leukemia, Adrenocortical Carcinoma, Bladder Urothelial Carcinoma, Brain Lower Grade Glioma, Breast Invasive Carcinoma, Cervical Squamous Cell Carcinoma and Endocervical Adenocarcinoma, Cholangiocarcinoma, Colon Adenocarcinoma, Lymphoid Neoplasm Diffuse Large B-cell Lymphoma, Esophageal Carcinoma, Glioblastoma Multiforme, Head and Neck Squamous Cell Carcinoma, Kidney Chromophobe, Kidney Renal Clear Cell Carcinoma, Kidney Renal Papillary Cell Carcinoma, Liver Hepatocellular Carcinoma, Lung Adenocarcinoma, Lung Squamous Cell Carcinoma, Mesothelioma, Ovarian Serous Cystadenocarcinoma, Pancreatic Adenocarcinoma, Pheochromocytoma and Paraganglioma, Prostate Adenocarcinoma, Rectum Adenocarcinoma, Sarcoma, Skin Cutaneous Melanoma, Stomach Adenocarcinoma, Testicular Germ Cell Tumors, Thyroid Carcinoma, Thymoma, Uterine Carcinosarcoma, Uterine Corpus Endometrial Carcinoma, Uveal Melanoma, or any combination thereof.

In embodiments, the invention provides a system configured to determine a presence or absence of metastatic cancer of a subject as described above/below, wherein the metastatic cancer comprises a cancer type, wherein the cancer type comprises: lung cancer, prostate cancer, melanoma cancer, breast cancer, thyroid cancer, or any combination thereof.

In embodiments, the invention provides a system configured to determine a presence or absence of metastatic cancer of a subject as described above/below, wherein the biological sample comprises a tissue sample, liquid biopsy, whole blood biopsy, or any combination thereof.

In embodiments, the invention provides a system configured to determine a presence or absence of metastatic cancer of a subject as described above/below, wherein the biological sample comprises constituents of whole blood comprising: plasma, white blood cells, red blood cells, platelets, or any combination thereof.

In embodiments, the invention provides a system configured to determine to a presence or absence of metastatic cancer of a subject as described above/below, wherein the machine-learning model is trained to discriminate between non-metastatic and metastatic cancerous tissue or blood samples.

In embodiments, the invention provides a system configured to determine a presence or absence of metastatic cancer of a subject as described above/below, wherein the machine-learning model is trained to differentiate one or more cancer types. The one or more cancer types may comprise: Acute Myeloid Leukemia, Adrenocortical Carcinoma, Bladder Urothelial Carcinoma, Brain Lower Grade Glioma, Breast Invasive Carcinoma, Cervical Squamous Cell Carcinoma and Endocervical Adenocarcinoma, Cholangiocarcinoma, Colon Adenocarcinoma, Lymphoid Neoplasm Diffuse Large B-cell Lymphoma, Esophageal Carcinoma, Glioblastoma Multiforme, Head and Neck Squamous Cell Carcinoma, Kidney Chromophobe, Kidney Renal Clear Cell Carcinoma, Kidney Renal Papillary Cell Carcinoma, Liver Hepatocellular Carcinoma, Lung Adenocarcinoma, Lung Squamous Cell Carcinoma, Mesothelioma, Ovarian Serous Cystadenocarcinoma, Pancreatic Adenocarcinoma, Pheochromocytoma and Paraganglioma, Prostate Adenocarcinoma, Rectum Adenocarcinoma, Sarcoma, Skin Cutaneous Melanoma, Stomach Adenocarcinoma, Testicular Germ Cell Tumors, Thyroid Carcinoma, Thymoma, Uterine Carcinosarcoma, Uterine Corpus Endometrial Carcinoma, Uveal Melanoma, or any combination thereof.

In embodiments, the invention provides a system configured to determine a presence or absence of metastatic cancer of a subject as described above/below, wherein the output further comprises an indication of type, tissue of origin, or any combination thereof the metastatic cancer.

In embodiments, the invention provides a method of broadly diagnosing metastatic cancer in a subject comprising: detecting microbial presence or abundance in a tissue or blood sample from the subject; determining that the detected microbial presence or abundance is different than microbial presence or abundance from one or more normal tissue sample(s) taken in the absence of a metastasis; and correlating the detected microbial to presence or abundance with a known microbial presence or abundance for a metastatic cancer, thereby diagnosing the metastatic cancer.

In embodiments, the invention provides a method of broadly diagnosing the tissue of origin of metastatic cancer in a subject comprising: detecting microbial presence or abundance in a tissue or blood sample from the subject with metastatic cancer; determining that the detected microbial presence or abundance is similar or different to the microbial presence or abundance in a population of previously studied subjects with primary tumors; and correlating the detected microbial presence or abundance of the metastatic cancer with the most similar primary tumor type, thereby diagnosing the tissue of origin of metastatic cancer.

In embodiments, the invention provides a method of diagnosing the tissue of origin of metastatic cancer in a subject comprising: detecting microbial presence or abundance in a liquid biopsy from the subject; determining that the detected microbial presence or abundance is similar or different to the microbial presence or abundance in one or more liquid biopsies from a population of healthy subjects and/or subjects with primary tumors; and correlating the detected microbial presence or abundance with the most similar liquid biopsies in this cohort, thereby diagnosing the presence or absence of the metastatic cancer, and, if present, its tissue of origin.

In embodiments, the invention provides a method of diagnosing the bodily location of metastatic cancer, wherein the location of origin is the bone (sarcoma), the adrenal glands, the bladder, the brain, the breast, the cervix, the gallbladder, the colon, the esophagus, the neck (head and neck squamous cell carcinoma), the kidney, the liver, the lung, the lymph nodes (diffuse large B-cell lymphoma), the skin, the ovary, the prostate, the rectum, the stomach, the thyroid, and the uterus, and wherein the subject is human.

In embodiments, the invention provides a method of diagnosing metastatic cancer, wherein the cancer is adrenocortical cancer, bladder cancer, brain cancer (lower grade glioma; glioblastoma), breast cancer, cervical cancer, cholangiocarcinoma, colon cancer, esophageal cancer, head and neck cancer, kidney cancer (chromophobe; renal clear cell carcinoma; papillary cell carcinoma), liver cancer, lung cancer (adenocarcinoma; squamous cell carcinoma), lymphoid neoplasm diffuse large B-cell lymphoma, melanoma (skin cutaneous melanoma, uveal melanoma), ovarian cancer, prostate cancer, rectum cancer, sarcoma, stomach cancer, thyroid cancer (thyroid carcinoma, thymoma), and uterine cancer, and wherein the subject is human.

In embodiments, the invention provides a method of predicting the molecular features of the human metastatic cancer using non-human features, wherein the molecular features are human mutations, wherein the non-human features are microbial presence or abundance.

In embodiments, the invention provides a method of predicting which subjects will respond or will not respond to a particular treatment for metastatic cancer, wherein the subject is human, wherein the treatment is immunotherapy, wherein the immunotherapy is a PD-1 blockade (e.g. nivolumab, pembrolizumab).

In embodiments, the invention provides a method of diagnosing metastatic cancer, further comprising treating the metastatic cancer in the subject based on the identified non-human features of the disease or the identified tissue of origin of the metastatic cancer, wherein the subject is human, wherein the non-human features are microbial presence or abundance.

In embodiments, the invention provides a method of diagnosing metastatic cancer, further comprising designing a new treatment to treat the metastatic cancer in the subject based on its non-human features, wherein the non-human features are microbial, wherein the subject is human.

In embodiments, the invention provides a method of diagnosing metastatic cancer, further distinguishing it from earlier stages of cancer in the subject based on its non-human features, wherein the non-human features are microbial, wherein the subject is human.

In embodiments, new treatments may be designed to target and exploit the non-human features associated with the metastatic cancer using one or more of the following modalities: small molecules, hormone therapies, biologics, engineered host-derived cell types, probiotics, engineered bacteria, natural-but-selective viruses, engineered viruses, and bacteriophages.

In embodiments, the invention provides a method of diagnosing metastatic cancer, further comprising longitudinal monitoring of its non-human features to indicate when a primary tumor metastasizes and/or when the disease responds to treatment, wherein the subject is human.

In embodiments, the invention provides a kit to measure the microbial presence or abundance in the metastatic cancer tissue or blood samples, thereby permitting diagnosis of the metastatic cancer and/or its tissue of origin.

In embodiments, the invention provides a computer system to analyze the microbial presence or abundance in the metastatic cancer tissue or blood samples and apply machine learning on this microbial presence or abundance, thereby making a diagnosis of the metastatic cancer and/or its tissue of origin.

In embodiments, the invention utilizes a diagnostic model based on a machine learning architecture.

In embodiments, the invention utilizes a diagnostic model based on a regularized machine learning architecture.

In embodiments, the invention utilizes a diagnostic model based on an ensemble of machine learning architectures.

In embodiments, the invention identifies and selectively removes certain non-human features as contaminants (“noise”) while selectively retaining other non-human features as non-contaminants (“signal”), wherein non-human features are microbial.

In embodiments, the invention provides a method of diagnosing metastatic cancer wherein the microbes are of bacterial, fungal, viral, archaeal, protozoal, and/or phage origin, or any combination thereof.

In embodiments, the invention provides a method of diagnosing metastatic cancer, wherein microbial presence or abundance information is combined with information about the subject and/or the subject's metastatic cancer to create a diagnostic model that has greater predictive performance than only having microbial presence or abundance information alone, wherein the subject is human.

In embodiments, the diagnostic model utilizes subject information in combination with microbial presence or abundance information from one or more of the following sources: immunohistochemistry protein markers of tumor tissue, tumor tissue DNA, tumor tissue RNA, tumor tissue methylation patterns, cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, or methylation patterns of circulating tumor cell derived RNA, methylation patterns of circulating tumor cell derived RNA.

In embodiments, microbial presence or abundance is detected by ecological shotgun sequencing, quantitative polymerase chain reaction, immunohistochemistry, in situ hybridization, flow cytometry, host whole genome sequencing, host transcriptomic sequencing, cancer whole genome sequencing, cancer transcriptomic sequencing, or any combination thereof, and/or wherein the microbial presence or abundance is detected using amplification of one or more of the following nucleic acid regions of microbial origin: V1, V2, V3, V4, V5, V6, V7, V8, or V9 variable domain region of 16S rRNA; or the internal transcribed spacer (ITS) region of the 18S rRNA, and/or wherein the microbial presence or abundance is detected by nucleic acid measurement that targets microbial DNA, RNA, or any combination thereof, wherein the measurement that targets microbial DNA, RNA, or any combination thereof occurs simultaneously with the measurement of host DNA, RNA, or any combination thereof.

In embodiments, the geospatial distribution of microbial presence or absence is measured in the metastatic cancer tissue of the host by one or more of the following methods: multisampling of the tumor tissue and/or its microenvironment, immunohistochemistry, in situ hybridization, digital spatial genomics, digital spatial to transcriptomics, or any combination thereof.

In embodiments, the microbial nucleic acids are detected simultaneously with nucleic acids from the host and subsequently distinguished.

In embodiments, the subject's nucleic acids are selectively depleted and the microbial nucleic acids are selectively retained prior to measurement (e.g., sequencing) of a combined nucleic acid pool, wherein the subject is human.

In embodiments, the microbial nucleic acids are selectively prior to measurement (e.g., sequencing) of a combined nucleic acid pool with the subject, wherein the subject is human.

In embodiments, the microbial and non-microbial nucleic acids are separated by aligning the nucleic acids against a reference database of microbial and non-microbial genomes.

In embodiments, microbial and non-microbial nucleic acids are separated without aligning the nucleic acids against a reference genome database.

In embodiments, the invention provides that the biological sample is blood, a constituent of blood (e.g., plasma), or a tissue biopsy, wherein the metastatic tissue biopsy is malignant or non-malignant, or any combination thereof.

In embodiments, the invention provides that the biological sample is a liquid biopsy, including but not limited to plasma, urine, saliva, or tears, or any combination thereof.

In embodiments, the microbial presence or abundance of the metastatic cancer is inferred by measuring microbial presence or abundance in other bodily locations of the subject's microbiome, wherein the subject is human.

In embodiments, the microbial presence or abundance in the biological sample of the subject is simultaneously informative of the presence and tissue of origin of the metastatic cancer.

In some embodiments, the disclosure describes a method of determining a treatment with at least 70% treatment efficacy of treating metastatic cancer of a subject, comprising: (a) detecting a microbial presence in a biological sample from the subject with metastatic cancer; (b) removing contaminated microbial features of the microbial presence, thereby producing a decontaminated microbial presence; (c) generating an association between the decontaminated microbial presence and the metastatic cancer of the subject; and (d) determining the treatment with at least 70% treatment efficacy of treating the metastatic cancer of the subject based on the association between the decontaminated microbial presence and the metastatic cancer. In some embodiments, the treatment comprises at least 80% or at least 90% treatment efficacy. In some embodiments, the treatment response comprises positive responder, non-responder, adverse responder, or any combination thereof. In some embodiments, the microbial presence further comprises a microbial abundance, wherein the microbial presence or abundance comprise the following non-mammalian domains of life: bacteria, fungi, viruses, archaea, protozoa, bacteriophages, or any combination thereof. In some embodiments, the contaminated microbial features comprise taxonomic assignment of the microbial presence. In some embodiments, step (b) is omitted. In some embodiments, the biological sample comprises a tissue sample, liquid biopsy, whole blood biopsy, or any combination thereof. In some embodiments, the biological sample comprises one or more constituents of whole blood comprising: plasma, white blood cells, red blood cells, platelets, or any combination thereof. In some embodiments, the treatment is not metabolized or rendered inactive by the decontaminated microbial presence. In some embodiments, the treatment comprises: a small molecule, a hormone therapy, a biologic, an engineered host-derived cell type or types, a probiotic, an engineered bacterium, a natural-but-selective virus, an engineered virus, a bacteriophage, or any combination thereof. In some embodiments, the metastatic cancer comprises: Acute Myeloid Leukemia, Adrenocortical Carcinoma, Bladder Urothelial Carcinoma, Brain Lower Grade Glioma, Breast Invasive Carcinoma, Cervical Squamous Cell Carcinoma and Endocervical Adenocarcinoma, Cholangiocarcinoma, Colon Adenocarcinoma, Lymphoid Neoplasm Diffuse Large B-cell Lymphoma, Esophageal Carcinoma, Glioblastoma Multiforme, Head and Neck Squamous Cell Carcinoma, Kidney Chromophobe, Kidney Renal Clear Cell Carcinoma, Kidney Renal Papillary Cell Carcinoma, Liver Hepatocellular Carcinoma, Lung Adenocarcinoma, Lung Squamous Cell Carcinoma, Mesothelioma, Ovarian Serous Cystadenocarcinoma, Pancreatic Adenocarcinoma, Pheochromocytoma and Paraganglioma, Prostate Adenocarcinoma, Rectum Adenocarcinoma, Sarcoma, Skin Cutaneous Melanoma, Stomach Adenocarcinoma, Testicular Germ Cell Tumors, Thyroid Carcinoma, Thymoma, Uterine Carcinosarcoma, Uterine Corpus Endometrial Carcinoma, Uveal Melanoma, or any combination thereof. In some embodiments, the treatment comprises an adjuvant given in combination with a primary treatment against the metastatic cancer to improve efficacy of the primary treatment. In some embodiments, the adjuvant is an antibiotic or an anti-microbial. In some embodiments, the treatment is based on microbial constituents or antigens associated with the metastatic cancer or the metastatic cancer's environment. In some embodiments, the treatment comprises an adoptive cell transfer to target microbial antigens, a cancer vaccine against microbial antigens, a monoclonal antibody against microbial antigens, an antibody-drug-conjugate designed to at least partially target microbial antigens, a multi-valent antibody, antibody fragment, antibody derivative thereof designed to at least partially target one or more microbial antigens, or any combination thereof. In some embodiments, the treatment comprises an antibiotic targeted against a class of functionally or biologically similar microbes of the microbial presence. In some embodiments, the treatment comprises two or more treatment types, wherein the two or more treatment types are combined such that at least one type of the two or more treatment types exploits the microbial presence or abundance associated with the metastatic cancer or the metastatic cancer environment to enhance therapeutic efficacy. In some embodiments, the association between the decontaminated microbial presence and the metastatic cancer further comprises the origin, type, or any combination thereof the metastatic cancer.

In some embodiments, the disclosure describes a method of predicting a treatment response of a metastatic cancer of a subject, comprising: (a) detecting a microbial presence in a biological sample from the subject with metastatic cancer; (b) removing contaminated microbial features of the microbial presence, thereby producing a decontaminated microbial presence; (c) generating an association between the decontaminated microbial presence and the metastatic cancer of the subject; and (d) predicting the treatment response of the metastatic cancer of the subject based the association between the decontaminated microbial presence and the metastatic cancer. In some embodiments, the treatment response comprises positive responder, non-responder, adverse responder, or any combination thereof. In some embodiments, the microbial presence further comprises a microbial abundance, wherein the microbial presence or abundance comprise the following non-mammalian domains of life: bacteria, fungi, viruses, archaea, protozoa, bacteriophages, or any combination thereof. In some embodiments, the contaminated microbial features comprise taxonomic assignment of the microbial presence. In some embodiments, step (b) is omitted. In some embodiments, the biological sample comprises a tissue sample, liquid biopsy, whole blood biopsy, or any combination thereof. In some embodiments, the biological sample comprises one or more constituents of whole blood comprising: plasma, white blood cells, red blood cells, platelets, or any combination thereof. In some embodiments, the treatment is not metabolized or rendered inactive by the decontaminated microbial presence. In some embodiments, the treatment comprises: a small molecule, a hormone therapy, a biologic, an engineered host-derived cell type or types, a probiotic, an engineered bacterium, a natural-but-selective virus, an engineered virus, a bacteriophage, or any combination thereof. In some embodiments, the metastatic cancer comprises: Acute Myeloid Leukemia, Adrenocortical Carcinoma, Bladder Urothelial Carcinoma, Brain Lower Grade Glioma, Breast Invasive Carcinoma, Cervical Squamous Cell Carcinoma and Endocervical Adenocarcinoma, Cholangiocarcinoma, Colon Adenocarcinoma, Lymphoid Neoplasm Diffuse Large B-cell Lymphoma, Esophageal Carcinoma, Glioblastoma Multiforme, Head and Neck Squamous Cell Carcinoma, Kidney Chromophobe, Kidney Renal Clear Cell Carcinoma, Kidney Renal Papillary Cell Carcinoma, Liver Hepatocellular Carcinoma, Lung Adenocarcinoma, Lung Squamous Cell Carcinoma, Mesothelioma, Ovarian Serous Cystadenocarcinoma, Pancreatic Adenocarcinoma, Pheochromocytoma and Paraganglioma, Prostate Adenocarcinoma, Rectum Adenocarcinoma, Sarcoma, Skin Cutaneous Melanoma, Stomach Adenocarcinoma, Testicular Germ Cell Tumors, Thyroid Carcinoma, Thymoma, Uterine Carcinosarcoma, Uterine Corpus Endometrial Carcinoma, Uveal Melanoma, or any combination thereof. In some embodiments, the treatment comprises an adjuvant given in combination with a primary treatment against the metastatic cancer to improve efficacy of the primary treatment. In some embodiments, the adjuvant is an antibiotic or an anti-microbial. In some embodiments, the treatment is based on microbial constituents or antigens associated with the metastatic cancer or the metastatic cancer's environment. In some embodiments, the treatment comprises an adoptive cell transfer to target microbial antigens, a cancer vaccine against microbial antigens, a monoclonal antibody against microbial antigens, an antibody-drug-conjugate designed to at least partially target microbial antigens, a multi-valent antibody, antibody fragment, antibody derivative thereof designed to at least partially target one or more microbial antigens, or any combination thereof. In some embodiments, the treatment comprises an antibiotic targeted against a class of functionally or biologically similar microbes of the microbial presence. In some embodiments, the treatment comprises two or more treatment types, wherein the two or more treatment types are combined such that at least one type of the two or more treatment types exploits the microbial presence or abundance associated with the metastatic cancer or the metastatic cancer environment to enhance therapeutic efficacy. In some embodiments, the association between the decontaminated microbial presence and the metastatic cancer further comprises the origin, type, or any combination thereof the metastatic cancer.

In some embodiments, the disclosure describes a method of determining an action during a course of treatment of a subject's metastatic cancer of a subject, comprising: (a) detecting a microbial presence in a biological sample from the subject with metastatic cancer; (b) removing contaminated microbial features of the microbial presence, thereby producing a decontaminated microbial presence; (c) generating an association between the decontaminated microbial presence and the metastatic cancer of the subject; and (d) determining the action during the course of the treatment of the subject's metastatic cancer based on the association between the decontaminated microbial presence and the metastatic cancer. In some embodiments, the action comprises discontinuing, beginning, or pausing the treatment of the subject's metastatic cancer. In some embodiments, the microbial presence further comprises a microbial abundance, wherein the microbial presence or abundance comprise the following non-mammalian domains of life: bacteria, fungi, viruses, archaea, protozoa, bacteriophages, or any combination thereof. In some embodiments, the contaminated microbial features comprise taxonomic assignment of the microbial presence. In some embodiments, step (b) is omitted. In some embodiments, the biological sample comprises a tissue sample, liquid biopsy, whole blood biopsy, or any combination thereof. In some embodiments, the biological sample comprises one or more constituents of whole blood comprising: plasma, white blood cells, red blood cells, platelets, or any combination thereof. In some embodiments, the treatment is not metabolized or rendered inactive by the decontaminated microbial presence. In some embodiments, the treatment comprises: a small molecule, a hormone therapy, a biologic, an engineered host-derived cell type or types, a probiotic, an engineered bacterium, a natural-but-selective virus, an engineered virus, a bacteriophage, or any combination thereof. In some embodiments, the metastatic cancer comprises: Acute Myeloid Leukemia, Adrenocortical Carcinoma, Bladder Urothelial Carcinoma, Brain Lower Grade Glioma, Breast Invasive Carcinoma, Cervical Squamous Cell Carcinoma and Endocervical Adenocarcinoma, Cholangiocarcinoma, Colon Adenocarcinoma, Lymphoid Neoplasm Diffuse Large B-cell Lymphoma, Esophageal Carcinoma, Glioblastoma Multiforme, Head and Neck Squamous Cell Carcinoma, Kidney Chromophobe, Kidney Renal Clear Cell Carcinoma, Kidney Renal Papillary Cell Carcinoma, Liver Hepatocellular Carcinoma, Lung Adenocarcinoma, Lung Squamous Cell Carcinoma, Mesothelioma, Ovarian Serous Cystadenocarcinoma, Pancreatic Adenocarcinoma, Pheochromocytoma and Paraganglioma, Prostate Adenocarcinoma, Rectum Adenocarcinoma, Sarcoma, Skin Cutaneous Melanoma, Stomach Adenocarcinoma, Testicular Germ Cell Tumors, Thyroid Carcinoma, Thymoma, Uterine Carcinosarcoma, Uterine Corpus Endometrial Carcinoma, Uveal Melanoma, or any combination thereof. In some embodiments, the treatment comprises an adjuvant given in combination with a primary treatment against the metastatic cancer to improve efficacy of the primary treatment. In some embodiments, the adjuvant is an antibiotic or an anti-microbial. In some embodiments, the treatment is based on microbial constituents or antigens associated with the metastatic cancer or the metastatic cancer's environment. In some embodiments, the treatment comprises an adoptive cell transfer to target microbial antigens, a cancer vaccine against microbial antigens, a monoclonal antibody against microbial antigens, an antibody-drug-conjugate designed to at least partially target microbial antigens, a multi-valent antibody, antibody fragment, antibody derivative thereof designed to at least partially target one or more microbial antigens, or any combination thereof. In some embodiments, the treatment comprises an antibiotic targeted against a class of functionally or biologically similar microbes of the microbial presence. In some embodiments, the treatment comprises two or more treatment types, wherein the two or more treatment types are combined such that at least one type of the two or more treatment types exploits the microbial presence or abundance associated with the metastatic cancer or the metastatic cancer environment to enhance therapeutic efficacy. In some embodiments, the association between the decontaminated microbial presence and the metastatic cancer further comprises the origin, type, or any combination thereof the metastatic cancer.

In some embodiments, the disclosure describes a method of creating a treatment to treat a subject's metastatic cancer, comprising: (a) detecting a microbial presence in a biological sample from the subject with metastatic cancer; (b) removing contaminated microbial features of the microbial presence, thereby producing a decontaminated microbial presence; (c) generating an association between the decontaminated microbial presence and the metastatic cancer of the subject; and (d) creating the treatment to treat the subject's metastatic cancer based on the association between the decontaminated microbial presence and the metastatic cancer. In some embodiments, the microbial presence further comprises a microbial abundance, wherein the microbial presence or abundance comprise the following non-mammalian domains of life: bacteria, fungi, viruses, archaea, protozoa, bacteriophages, or any combination thereof. In some embodiments, the contaminated microbial features comprise taxonomic assignment of the microbial presence. In some embodiments, step (b) is omitted. In some embodiments, the biological sample comprises a tissue sample, liquid biopsy, whole blood biopsy, or any combination thereof. In some embodiments, the biological sample comprises one or more constituents of whole blood comprising: plasma, white blood cells, red blood cells, platelets, or any combination thereof. In some embodiments, the treatment is not metabolized or rendered inactive by the decontaminated microbial presence. In some embodiments, the treatment comprises: a small molecule, a hormone therapy, a biologic, an engineered host-derived cell type or types, a probiotic, an engineered bacterium, a natural-but-selective virus, an engineered virus, a bacteriophage, or any combination thereof. In some embodiments, the metastatic cancer comprises: Acute Myeloid Leukemia, Adrenocortical Carcinoma, Bladder Urothelial Carcinoma, Brain Lower Grade Glioma, Breast Invasive Carcinoma, Cervical Squamous Cell Carcinoma and Endocervical Adenocarcinoma, Cholangiocarcinoma, Colon Adenocarcinoma, Lymphoid Neoplasm Diffuse Large B-cell Lymphoma, Esophageal Carcinoma, Glioblastoma Multiforme, Head and Neck Squamous Cell Carcinoma, Kidney Chromophobe, Kidney Renal Clear Cell Carcinoma, Kidney Renal Papillary Cell Carcinoma, Liver Hepatocellular Carcinoma, Lung Adenocarcinoma, Lung Squamous Cell Carcinoma, Mesothelioma, Ovarian Serous Cystadenocarcinoma, Pancreatic Adenocarcinoma, Pheochromocytoma and Paraganglioma, Prostate Adenocarcinoma, Rectum Adenocarcinoma, Sarcoma, Skin Cutaneous Melanoma, Stomach Adenocarcinoma, Testicular Germ Cell Tumors, Thyroid Carcinoma, Thymoma, Uterine Carcinosarcoma, Uterine Corpus Endometrial Carcinoma, Uveal Melanoma, or any combination thereof. In some embodiments, the treatment comprises an adjuvant given in combination with a primary treatment against the metastatic cancer to improve efficacy of the primary treatment. In some embodiments, the adjuvant is an antibiotic or an anti-microbial. In some embodiments, the treatment is based on microbial constituents or antigens associated with the metastatic cancer or the metastatic cancer's environment. In some embodiments, the treatment comprises an adoptive cell transfer to target microbial antigens, a cancer vaccine against microbial antigens, a monoclonal antibody against microbial antigens, an antibody-drug-conjugate designed to at least partially target microbial antigens, a multi-valent antibody, antibody fragment, antibody derivative thereof designed to at least partially target one or more microbial antigens, or any combination thereof. In some embodiments, the treatment comprises an antibiotic targeted against a class of functionally or biologically similar microbes of the microbial presence. In some embodiments, the treatment comprises two or more treatment types, wherein the two or more treatment types are combined such that at least one type of the two or more treatment types exploits the microbial presence or abundance associated with the metastatic cancer or the metastatic cancer environment to enhance therapeutic efficacy. In some embodiments, the association between the decontaminated microbial presence and the metastatic cancer further comprises the origin, type, or any combination thereof the metastatic cancer.

### BRIEF DESCRIPTION OF THE DRAWINGS

FIG. 1 shows leave-one-out-cross-validation (LOOCV) machine learning results that discriminate metastatic breast cancer and metastatic thyroid carcinoma tissue samples, thereby diagnosing the primary tumor of origin, by its tissue microbiome in 18 subjects (since metastatic cancers are named by their tissue of origin).

FIG. 2 shows an analysis predicting metastatic cancers vs. non-metastatic cancers using blood-borne microbial DNA from 559 subjects.

FIG. 3 shows discrimination between metastatic melanoma and other metastatic cancer types using blood-based microbial DNA from 15 subjects. Samples labeled “Other metastatic cancer types” depicted in FIG. 3 include breast cancer (2 samples), metastatic thyroid cancer (2 samples), and metastatic esophageal cancer (1 sample). In various embodiments, these cancer types and/or other combinations may be combined to provide sufficient numbers to test.

FIG. 4a illustrates a lollipop plot showing Lollipop plot showing the percentage of total sequencing reads identified by the microbial-detection pipeline, and those resolved at the genus level in TCGA data set by Kraken. LAML, acute myeloid leukemia; PAAD, pancreatic adenocarcinoma; GBM, glioblastoma multiforme; PRAD, prostate adenocarcinoma; ESCA, esophageal carcinoma; TCGT, testicular germ cell tumors; BRCA, breast invasive carcinoma; THCA, thyroid carcinoma; KICH, kidney chromophobe; THYM, thymoma; READ, rectum adenocarcinoma; SARC, sarcoma; UVM, uveal melanoma; CHOL, cholangiocarcinoma; ACC, adrenocortical carcinoma; UCEC, uterine corpus endometrial carcinoma; LUSC, lung squamous cell carcinoma; PCPG, pheochromocytoma and paraganglioma; BLCA, bladder urothelial carcinoma; UCS, uterine carcinosarcoma; LGG, brain lower grade glioma (FIG. 4a). The total number of samples included across all cancer types is 17,625. FIG. 4b illustrates a CONSORT-style diagram showing quality control processing and the number of remaining samples. FFPE, fixed-formalin paraffin-embedded. FIG. 4c illustrates principal components analysis (PCA) of Voom-normalized data, with cancer microbiome samples colored by sequencing center. FIG. 4d illustrates PCA of Voom-SNM data. FIG. 4e illustrates principal variance components analysis of raw taxonomical count data, Voom-normalized data, and Voom-SNM data. FIGS. 4f-h illustrate heatmaps of classifier performance metrics (AUROC (ROC) and AUPR (PR)) from greyscale-red (high) to greyscale-blue (low) for distinguishing between TCGA primary tumors (FIG. 4f), between tumor and normal samples (FIG. 4g), and between stage I and stage IV cancers (FIG. 4h). “NA” may indicate that not enough samples (e.g., fewer than 20) were available in any ML class for model training.

FIGS. 5a-g illustrates ecological validation of viral and bacterial reads within the TCGA cancer microbiome data set, according to at least one embodiment. FIG. 5a illustrates average body site attribution for solid-tissue normal samples from patients with COAD (n=70) using Source Tracker2 trained on the HMP2 data set. FIG. 5b illustrates differential abundances of the Fusobacterium genus for common gastrointestinal (GI) cancers associated with Fusobacterium spp. BDN, blood derived normal; STN, solid tissue normal; PT, primary tumor. FIG. 5c illustrates differential abundances of Fusobacterium among grouped GI cancers (n=8: COAD, READ, CHOL, LIHC, PAAD, HNSC, ESCA, STAD; for abbreviations see FIG. 8a) and non-GI cancers (n=24) (see Methods). FIGS. 5d-e illustrate normalized HPV abundances for HPV infected patients with CESC (FIG. 5d) or HNSC (FIG. 5e), as clinically denoted in TCGA. ISH, in situ hybridization; IHC, immunohistochemistry. FIG. 5f illustrates normalized Orthohepadnavirus abundance in patients with LIHC with clinically adjudicated risk factors: HepB, prior hepatitis B infection; EtOH, heavy alcohol consumption; HepC, prior hepatitis C infection. FIG. 5g illustrates Normalized EBV abundance in STAD integrative molecular subtypes: CIN, chromosomal instability; GS, genome stable; MSI, microsatellite unstable; EBV, EBV-infected samples. In all panels, blood-derived normal and/or solid-tissue normal data are shown as comparative negative controls; two-sided Mann-Whitney U-tests were used with multiple testing correction for more than two comparisons; box plots show median (line), 25th and 75th percentiles (box), and 1.5× the interquartile range (IQR, whiskers). Greyscale-blue numbers show sample sizes for each group.

FIGS. 6a-d illustrates classifier performance for cancer discrimination using mbDNA in blood and as a complementary diagnostic approach for cancer ‘liquid’ biopsies. FIG. 6a illustrates model performance heatmap analogous to FIGS. 4f-h to predict one cancer type versus all others using blood mbDNA with TCGA study IDs on the right (FIG. 8a); at least 20 samples were required in each ML minority class to be eligible.

FIG. 6b illustrates ML model performances predicting one cancer type versus all others using blood mbDNA for stage Ia-IIc cancers. FIGS. 6c-d illustrate ML model performances using blood mbDNA from patients without detectable primary tumor genomic alterations, per Guardant360 (FIG. 6c) and FoundationOne Liquid (FIG. 6d) ctDNA assays. FD, full data; LCR, likely contaminants removed by sequencing center; APCR, all putative contaminants removed by sequencing center; PCCR, plate-center contaminants removed; MSF, most stringent filtering by sequencing center. The number of samples included to evaluate the performance of each comparison can be found in the data browser confusion matrices at cancermicrobiome.ucsd.edu/CancerMicrobiome_DataBrowser.

FIGS. 7a-k illustrates performance of ML models to discriminate between types of cancer and healthy controls using plasma-derived, cell-free mbDNA. FIG. 7a, Demographics of samples analyzed in the validation study. All patients had high-grade (stage III-IV) cancers of multiple subtypes and were aggregated into PC, LC, and SKCM groups. FIG. 7b illustrates bootstrapped performance estimates for distinguishing grouped cancer samples (n=100) from non-cancer healthy controls (n=69). Rasterized density plot of ROC (top) and PR (bottom) curve data from 500 iterations with different training—testing splits (70% training-30% testing). FIGS. 7c-h illustrate leave-one-out (LOO) iterative ML performances between two classes: prostate cancer (PC) versus control (Ctrl; FIG. 7c), lung cancer (LC) versus control (FIG. 7d), melanoma (SKCM) versus control (FIG. 7e), PC versus LC (FIG. 7f), LC versus SKCM (FIG. 7g), and PC versus SKCM (FIG. 7h). FIGS. 7i-k illustrate multi-class (n=3 or 4), LOO iterative ML performances to distinguish among types of cancer (FIG. 7i) and between mixed patients with cancer and healthy control individuals (FIG. 7j and FIG. 7k respectively). Overall LOO ML performance was calculated as the mean of performances when comparing one versus all others (shown below the confusion matrices).

FIGS. 8a-g illustrates continued overview of the TCGA cancer microbiome. FIG. 8a illustrates a table of TCGA study abbreviations. FIG. 8b illustrates PCA of Voom-normalized data, where greyscale-colors represent sequencing platform of the sample and each dot denotes a cancer microbiome sample. FIG. 8c illustrates PCA of the data following consecutive Voom-SNM supervised normalization, as labelled by sequencing platform. FIG. 8d illustrates PCA of Voom-normalized data, where greyscale-colors represent experimental strategy of the sample and each dot denotes a cancer microbiome sample. FIG. 8e illustrates PCA of the data following consecutive Voom-SNM supervised normalization, as labelled by experimental strategy. FIGS. 8f-g illustrate microbial reads counts as normalized by the quantity of samples within a given sample type across all types of cancer in TCGA after metadata quality control (FIG. 4b), including the three major sample types analyzed in the paper (FIG. 8f) and the remaining sample types (FIG. 8g). ANP, additional, new primary; AM, additional metastatic; MM, metastatic; RT, recurrent tumor. For PCAs of raw and normalized data, n=17,625.

FIGS. 9a-h illustrate performance metrics discriminating between and within TCGA types of cancer using microbial abundances. FIGS. 9a-f illustrate examples from the heatmaps in FIGS. 4f-h. A greyscale-color gradient (top) denotes the probability threshold at any point along the ROC and PR curves. An inset confusion matrix is shown using a 50% probability threshold cutoff, which can be used to calculate sensitivity, specificity, precision, recall, positive predictive value, negative predictive values, and so forth at the corresponding point on the ROC and PR curves. FIGS. 9g-h illustrate linear regressions of model performance, specifically AUROC (FIG. 9g) and AUPR (FIG. 9h), for discriminating between types of cancer in a one-cancer-type-versus-all-others manner, as a function of minority class size. Performances are shown for models using microorganisms detected in primary tumors, with the greatest number of samples (n=13,883) and types of cancer (n=32) to compare. As AUROC and AUPR have domains of [0,1] and the minority class size varied from 20 to 1,238 samples, the latter is regressed on a log₁₀ scale. Inset hypothesis tests and associated P values are based on the null hypothesis of there being no relationship between the dependent and independent variables (two-sided hypothesis test of slope). The number of samples included to evaluate performance of each comparison can be found in the data browser confusion matrices at cancermicrobiome.ucsd.edu/CancerMicrobiome_DataBrowser.

FIGS. 10a-i illustrate internal validation of a ML model pipeline. FIG. 10a illustrates two independent halves of TCGA raw microbial count data were normalized and used for model training to predict one cancer type versus all others using tumor microbial DNA and RNA; each model was then applied to the other half's normalized data. This heatmap compares the performances of these models compared to training and testing on 50-50% splits of the full data set (split 1: n=8,814 samples; split 2: n=8,811 samples; total samples: n=17,625). FIGS. 10b-c illustrate model performance comparison when subsetting the full Voom-SNM data by primary tumor RNA samples (n=11,741) across multiple sequencing centers to predict one cancer type versus all others (FIG. 10b, AUROC; FIG. 10c, AUPR). FIGS. 10d-e illustrate model performance comparison when subsetting the full Voom-SNM data by primary tumor DNA samples (n=2,142) across multiple sequencing centers to predict one cancer type versus all others (FIG. 10d, AUROC; FIG. 10e, AUPR). FIGS. 10f-g illustrate model performance comparison when subsetting the full Voom-SNM data by samples from the UNC (n=9,726), which only did RNA-seq, to predict one cancer type versus all others using primary tumor RNA samples (FIG. 10f, AUROC; FIG. 10g, AUPR). FIGS. 10h-i illustrate model performance comparison when subsetting the full Voom-SNM data by samples from HMS (n=898), which only did WGS, to predict one cancer type versus all others using primary tumor DNA samples (FIG. 10h, AUROC; FIG. 10i, AUPR). FIGS. 10b-i, illustrate generalized linear models with s.e. are shown in grey; dotted diagonal line denotes a perfect linear relationship; for sample size comparison, the full Voom-SNM data set contained 13,883 primary tumor samples.

FIGS. 11a-t illustrate orthogonal validation of Kraken-derived TCGA cancer microbiome profiles and their ML performances. FIGS. 11a-h illustrate four TCGA types of cancer (CESC, n=142 (DNA) and n=309 (RNA); STAD, n=322 (DNA) and n=770 (RNA); LUAD, n=351 (DNA) and n=600 (RNA); and OV, n=189 (DNA) and n=850 (RNA)) underwent additional filtering after Kraken-based taxonomy assignments via direct genome alignments (BWA) using tumor microbial DNA and RNA. ML performances are compared between the normalized, BWA filtered data and matched, independently normalized Kraken data for one cancer type versus all others using primary tumor microorganisms (FIG. 11a, AUROC; Figure lib, AUPR), tumor-versus-normal discriminations (FIG. 11c, AUROC; Figure lid, AUPR), stage I versus stage IV tumor discriminations using primary tumor microorganisms (FIG. 11e, AUROC; FIG. 11f, AUPR), and one cancer type versus all others using blood-derived microorganisms (FIG. 11g, AUROC; FIG. 11h, AUPR) (see Methods). FIG. 11i illustrates Venn diagram of the taxon count between the BWA filtered data and the Kraken full data. FIGS. 11j-t illustrate an orthogonal microbial-detection pipeline called SHOGUN and a separate database were run on a subset of TCGA samples (n=13,517 total samples), normalized via Voom-SNM, analogous to its Kraken counterpart, and used for downstream ML analyses.

FIG. 11j, Venn diagram of the SHOGUN-derived microbial taxa (S) and the Kraken-derived microbial taxa (K). Note that SHOGUN's database does not include viruses whereas the Kraken database does. FIGS. 11k-l illustrate PCA of Voom (FIG. 11k) and Voom-SNM (FIG. 11l) normalized SHOGUN data, greyscale-colored by sequencing center. FIGS. 11m-t illustrate ML performance comparisons between models trained and tested on SHOGUN data and matched Kraken data, using the same 70%-30% splits, for one cancer type versus all others using primary tumor microorganisms (FIG. 11m, AUROC; FIG. 11n, AUPR), tumor-versus-normal discriminations (FIG. 11o, AUROC; FIG. 11p, AUPR), stage I versus stage IV tumor discriminations using primary tumor microorganisms (FIG. 11q, AUROC; FIG. 11r, AUPR), and one cancer type versus all others using blood-derived microorganisms (FIG. 11s, AUROC; FIG. 11t, AUPR). For fair comparison, matched Kraken data were derived by removing all virus assignments in the raw Kraken count data and subsetting to the same 13,517 TCGA samples analyzed by SHOGUN; these matched Kraken data were then normalized independently via Voom-SNM in the same way as the SHOGUN data (see Methods) and fed into downstream ML pipelines. For all ML performances, ≥20 samples in each class was required to be eligible. For regression subfigures, the dotted diagonal line denotes perfect performance correspondence; generalized linear models with s.e. ribbons are shown.

FIGS. 12a-e illustrate pan-cancer microbial abundances and an interactive website for TCGA cancer microbiome profiling and ML model inspection. FIG. 12a illustrate pan-cancer normalized abundances of Fusobacterium with a one-way ANOVA (Kruskal-Wallis) test for microbial abundances across types of cancer for each sample type. Sample sizes are inset in greyscale-blue and box plots show median (line), 25th and 75th percentiles (box), and 1.5×IQR (whiskers); TCGA study abbreviations are listed below and defined in FIG. 8a. FIG. 12b illustrates SourceTracker2 results for fecal contribution, as based on HMP2 data, for TCGA-COAD solid-tissue normal samples (n=70) and TCGASKCM primary tumor samples (n=122). Only one solid tissue normal sample was available for TCGA-SKCM (Supplementary Table 4), so primary tumors were used instead as the best proxy of expected skin flora. It is expected that colon samples should have higher fecal contribution than skin, so a one-sided Mann-Whitney U-test was used. As SourceTracker2 outputs the mean fractional contributions of each source (that is, HMP2) to each sink (that is, COAD, SKCM samples), the center value of each bar plot is the mean of these values and the error bars denote the s.e.m. The sample sizes are shown below in grey scale-blue. FIG. 12c illustrates pan-cancer normalized abundances of Alphapapillomavirus with a one-way ANOVA (Kruskal-Wallis) test for microbial abundances across types of cancer for each sample type. Sample sizes are inset in greyscale-blue, and box plots show median (line), 25th and 75th percentiles (box), and 1.5×IQR (whiskers); TCGA study abbreviations are listed below and defined in FIG. 8a. TCGA studies that clinically tested patients for HPV infection are divided into negative and positive groups. FIG. 12d illustrates screenshot of interactive website showing plotting of Alphapapillomavirus normalized microbial abundances using Kraken-derived data. Plotting using SHOGUN-derived normalized microbial abundances is available on another tab of the website (left-hand side). FIG. 12e illustrates screenshot of interactive website of ML model inspection. Selecting the data type (for example, all likely contaminants removed), cancer type (for example, invasive breast carcinoma), and comparison of interest (for example, tumor versus normal) will automatically update the ROC and PR curves, as well as the confusion matrix (using a probability cutoff threshold of 50%) and the ranked model feature list. Website is accessible at cancermicrobiome.ucsd.edu/CancerMicrobiome_DataBrowser.

FIGS. 13a-l illustrate decontamination approach along with its results, benefits, and limitations on cancer microbiome data. FIG. 13a illustrates various approaches used to evaluate, mitigate, remove and/or simulate sources of contamination. FIG. 13b illustrates the proportion of remaining taxa or microbial reads in TCGA after varying levels of decontamination. Decontamination by sequencing center removed all taxa identified as a contaminant at any one sequencing center (n=8 batches); decontamination by plate-center combinations removed all taxa identified as a contaminant on any single sequencing plate with more than ten TCGA samples on it (n=351 batches). FIGS. 13c-f illustrate body-site attribution prediction on the likely contaminants removed data set (FIG. 13c), the plate-center decontaminated data set (FIG. 13d), the all putative contaminants removed data set (FIG. 13e), and the most stringent filtering data set (FIG. 13f). FIGS. 13g-l illustrate models and concomitant performance values (AUROC and AUPR) were re-generated using the four decontaminated data sets described above (each labelled with a different greyscale-color as shown above). The AUROC and AUPR values obtained from models trained and tested on the decontaminated data sets are plotted against the AUROC or AUPR values from the full data set (FIGS. 4f-h). The dashed diagonal line denotes a perfect linear relationship. Generalized linear models have been fitted to the AUROC and AUPR values of the corresponding data sets; s.e. of the linear fits are shown by the associated shaded regions. COAD (n=1,006 total samples) model performances are identified throughout the Figures.

FIGS. 14a-c illustrate decontamination effects on proportion of average reads per sample type. The total read count (DNA and RNA) of each major sample type (primary tumor (FIG. 14a), solid-tissue normal (FIG. 14b), blood-derived normal (FIG. 14c)) was summed and divided by the total number of samples within each sample type. This normalized read count (per sample type) was then divided by the summed normalized read count across all sample types for each cancer type, thereby providing an estimate of the proportion of average reads per sample type per cancer type. This was repeated for all five data sets, as shown by the legend, to assess whether decontamination differentially impacted certain types of sample and/or cancer; relative stability in the percentages shown would suggest a lack of differential contamination. Minor sample types that were not further analyzed in this paper by decontamination or ML (for example, additional metastatic lesions; n=4 sample types; FIG. 8g) are not shown here. Note, in the special case that only one sample type existed for a given cancer type (primary tumor in ACC, MESO, UCS), then all bars will show that 100% of the normalized reads came from that one sample type. The number of total cancer samples examined is 17,625.

FIGS. 15a-e illustrates measuring spiked pseudo-contaminant contribution in downstream ML models and theoretical sensitivities of commercially available, host-based, ctDNA assays in patients from TCGA. FIGS. 15a-b illustrate feature importance scores were calculated for all taxa used in models trained to discriminate one cancer type versus all others in all four decontaminated data sets (FIG. 13b) using primary tumor microbial DNA or RNA (FIG. 15a), or using blood-derived mbDNA (FIG. 15b). These decontaminated data sets were spiked with pseudo-contaminants before the decontamination and normalization pipelines to evaluate their performance (see Methods), and the test set performances of the models shown are given in FIGS. 13g-h and FIG. 6a, respectively. Any spiked pseudo-contaminant(s) used by a model had their feature importance score(s) divided by the sum total of all feature importance scores in that model to estimate their percentage contribution towards making accurate predictions; the higher the score (out of 100), the less biologically reliable the model is. Note, zero means that no spiked pseudocontaminants were used for making predictions by the model; none of the models generated on the plate-center decontaminated data included spiked pseudo-contaminants as features. The number of samples included to evaluate performance of each comparison can be found in the data browser confusion matrices at cancermicrobiome.ucsd.edu/CancerMicrobiome_DataBrowser. FIGS. 15c-d illustrate percentage distribution among TCGA studies of patients with one or more genomic alterations on FoundationOne Liquid ctDNA coding genes (FIG. 15c) or on Guardant360 ctDNA coding genes (FIG. 15d). The number of samples examined and raw data are available at cbioportal.org. FIG. 15e illustrates a table comprising a list of coding genes for the FoundationOne and Guardant360 ctDNA assays and their examined alterations (source listed in the Methods).

FIGS. 16a-k illustrate analysis for real-world, plasma-derived, cell-free microbial DNA analysis between and among healthy individuals and multiple types of cancer. FIG. 16a illustrates discriminatory simulations in TCGA used to empirically power the real-world validation study (FIG. 7; see Methods). Center values for each stratified sample size are the means of the performances across ten iterations; error bars denote s.e.m. FIG. 16b illustrates evaluation of Aliivibrio genus abundance values (raw read counts) among positive control bacterial (Aliivibrio) monocultures, negative control blanks, and human sample types using Kraken and SHOGUN-derived data. FIG. 16c illustrates Aliivibrio genus abundance (raw read counts) across bacterial monoculture dilutions. FIG. 16d illustrates age distribution among cancer-free healthy control individuals (Ctrl) and grouped patients with lung cancer (LC), prostate cancer (PC), or melanoma (SKCM). FIG. 16e illustrates gender distribution among patients with inset Pearson's χ² test (one-sided critical region). FIG. 16f illustrates Venn diagram of taxon assignments between Kraken and SHOGUN, which used different databases. FIG. 16g illustrates iterative LOO ML regression of host age using Kraken (greyscale-pink) or SHOGUN (greyscale-aqua) raw microbial count data in healthy cancer-free individuals. Mean absolute errors (MAE) evaluated across all samples are shown. FIGS. 16h-j illustrate the effects of permuted age (FIG. 16h), sex (FIG. 16i), and age and sex (FIG. 16j) before Voom-SNM on ML performance to discriminate healthy individuals versus grouped patients with cancer using cell-free microbial DNA. One hundred permutations were used for each comparison (see Methods). FIG. 16k illustrates iterative subsampling of prostate cancer (PC), lung cancer (LC), melanoma (SKCM), and control groups to match SKCM cohort size (n=16 samples), followed by leave-one-out (LOO) pairwise ML of each subsampled cancer type against subsampled healthy controls. One hundred permuted iterations were used to estimate discriminatory performance distributions and standard errors (see Methods). Relating to FIGS. 16b-c, note the log₁₀ scale and 0.5 pseudo-count lower limit (dotted line). Relating to FIGS. 16b-d, h-k, hypothesis tests are two-sided Mann-Whitney U-tests with multiple testing correction when testing more than two comparisons; box plots show median (line), 25th and 75th percentiles (box), and 1.5× IQR (whiskers). For all box plots and bar plots, sample sizes are shown in greyscale-blue below.

FIGS. 17a-j illustrates SHOGUN-derived ML performances to discriminate between types of cancer and healthy, cancer-free individuals using cell-free microbial DNA. FIG. 17a illustrates bootstrapped performance estimates for distinguishing grouped patients with cancer (n=100) from cancer-free healthy control individuals (n=69). ROC and PR curve data from 500 iterations with different training— testing splits (70% training-30% testing) are shown on the rasterized density plot; mean values and 95% CI estimates are shown. FIG. 17b-g illustrate LOO iterative ML performance between two classes: prostate cancer (PC) versus control (FIG. 17b), lung cancer (LC) versus control (FIG. 17c), melanoma (SKCM) versus control (FIG. 17d), PC versus LC (FIG. 17e), LC versus SKCM (FIG. 17f), and PC versus SKCM (FIG. 17g). FIGS. 17h-j illustrate multi-class (n=3 or 4), leave-one-out (LOO) iterative ML performances to distinguish between types of cancer, as well as between patients with cancer and healthy cancer-free control individuals. Mean AUROC and AUPR, as calculated from one-versus-all-others AUROC and AUPR values, are shown below the confusion matrices. FIG. 17h illustrates LOO ML performance between the three types of cancer under study. FIG. 17i illustrates LOO ML performance between the three sample types with at least 20 samples in the minority class (that is, the cutoff used in the TCGA analysis, FIGS. 4f-h). FIG. 17j illustrates LOO ML performance between all four sample types under study. For all subfigures with confusion matrix plots: LOO ML was used instead of single or bootstrapped training—testing splits because of small sample sizes; these confusion matrices also reflect the number of samples used for each comparison.

FIG. 18 is a block diagram illustrating an example of a computing device or computer system upon which any of one or more techniques (e.g., methods) may be performed, in accordance with one or more example embodiments of the present disclosure.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of various embodiments will employ, unless otherwise indicated, conventional techniques of molecular biology (including diagnostic techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams to & Wilkins 2003), and Remington, The Science and Practice of Pharmacy, 22th ed., (Pharmaceutical Press and Philadelphia College of Pharmacy at University of the Sciences 2012).

At least one embodiment provides methods for the detection and determination of a metastasis tissue of origin on the basis of microbiota in tissue or blood of a subject with metastatic cancer. In embodiments, the invention provides a method for determining a metastasis's tissue of origin on the basis of microbiota in tissue or blood using microbial nucleic acids comprising:
(a) obtaining a sample of metastatic cancer tissue from a patient biopsy, including solid tissues or blood;
(b) extract nucleic acid from the sample of cancer tissue, for example with the ZymoBIOMICS DNA Miniprep Kit;
(c) preparing nucleic acid sequencing libraries from the extracted nucleic acid, such as using the KAPA HyperPlus Kit;
(d) sequencing the nucleic acid libraries using next generation sequencing (NGS), such as on an Illumina NovaSeq 6000 instrument;
(e) aligning outputted nucleic acid sequencing reads against known microbial genomes to obtain a table of microbial abundances for the sample; such as using the SHOGUN algorithm (PMID: 30443602); and
(f) inputting the table of microbial abundances into a machine learning algorithm in order to obtain a determination or prediction of the metastatic cancer's tissue of origin, such as using gradient boosting classification trees.

At least one embodiment provides that the nucleic acid can be DNA or RNA. In embodiments, the steps can be used with a focus on microbial DNA or RNA. Other alternatives include combinations of microbial DNA and RNA with host DNA and RNA to make a more accurate diagnosis of the metastasis's tissue of origin.

At least one embodiment provides that non-microbial nucleic acids are removed prior to aligning nucleic acid sequencing reads against known microbial genomes.

At least one embodiment provides that contaminating microbial nucleic acids are removed prior to aligning nucleic acid sequencing reads against known microbial genomes.

At least one embodiment provides that contaminating microbial nucleic acids are removed after aligning nucleic acid sequencing reads against known microbial genomes but before inputting the table of microbial abundances into a machine learning algorithm.

At least one embodiment generates microbial presence or absence information when aligning outputted nucleic acid sequencing reads against known microbial genomes, wherein the microbial presence or absence information is later used for machine learning.

At least one embodiment provides that the nucleic acid can be extracted from any tissues of the subject, including solid tissue, tumors, blood, a liquid biopsy, or any combination thereof. The nucleic acids therefore may be extracted from circulating blood, constituents of circulating blood (e.g., plasma, white blood cells, platelets), or any combination thereof.

At least one embodiment further provides methods of prognosing, preventing a procedure, and/or treating a subject based on the determination of the tissue of origin of the metastatic cancer, comprising administering to the subject an effective amount of a therapeutic composition or treatment protocol indicated for the metastasis.

### Definitions

To facilitate understanding of the invention, a number of terms and abbreviations as used herein are defined below as follows:

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of to the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or animal subject to be diagnosed or treated.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfate; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

“Amplification” refers to any known procedure for obtaining multiple copies of a target nucleic acid or its complement, or fragments thereof. The multiple copies may be referred to as amplicons or amplification products. Amplification, in the context of fragments, refers to production of an amplified nucleic acid that contains less than the complete target nucleic acid or its complement, e.g., produced by using an amplification oligonucleotide that hybridizes to, and initiates polymerization from, an internal position of the target nucleic acid. Known amplification methods include, for example, replicase-mediated amplification, polymerase chain reaction (PCR), reverse transcription polymerase chain reaction (RT-PCR), ligase chain reaction (LCR), strand-displacement amplification (SDA), and transcription-mediated or transcription-associated amplification. Amplification is not limited to the strict duplication of the starting molecule. For example, the generation of multiple cDNA molecules from RNA in a sample using reverse transcription (RT)-PCR is a form of amplification. Furthermore, the generation of multiple RNA molecules from a single DNA molecule during the process of transcription is also a form of amplification. During amplification, the amplified products can be labeled using, for example, labeled primers or by incorporating labeled nucleotides.

“Amplicon” or “amplification product” refers to the nucleic acid molecule generated during an amplification procedure that is complementary or homologous to a target nucleic acid or a region thereof. Amplicons can be double stranded or single stranded and can include DNA, RNA or both. Methods for generating amplicons are known to those skilled in the art.

“Codon” refers to a sequence of three nucleotides that together form a unit of genetic code in a nucleic acid.

“Codon of interest” refers to a specific codon in a target nucleic acid that has diagnostic or therapeutic significance (e.g. an allele associated with viral genotype/subtype or drug resistance).

“Complementary” or “complement thereof” means that a contiguous nucleic acid base sequence is capable of hybridizing to another base sequence by standard base pairing (hydrogen bonding) between a series of complementary bases. Complementary sequences may be completely complementary (i.e. no mismatches in the nucleic acid duplex) at each position in an oligomer sequence relative to its target sequence by using standard base pairing (e.g., G:C, A:T or A:U pairing) or sequences may contain one or more positions that are not complementary by base pairing (e.g., there exists at least one mismatch or unmatched base in the nucleic acid duplex), but such sequences are sufficiently complementary because the entire oligomer sequence is capable of specifically hybridizing with its target sequence in appropriate hybridization conditions (i.e. partially complementary). Contiguous bases in an oligomer are typically at least 80%, preferably at least 90%, and more preferably completely complementary to the intended target sequence.

“Configured to” or “designed to” denotes an actual arrangement of a nucleic acid sequence configuration of a referenced oligonucleotide. For example, a primer that is configured to generate a specified amplicon from a target nucleic acid has a nucleic acid sequence that hybridizes to the target nucleic acid or a region thereof and can be used in an amplification reaction to generate the amplicon. Also as an example, an oligonucleotide that is configured to specifically hybridize to a target nucleic acid or a region thereof has a nucleic acid sequence that specifically hybridizes to the referenced sequence under stringent hybridization conditions.

“Downstream” means further along a nucleic acid sequence in the direction of sequence transcription or read out.

“Upstream” means further along a nucleic acid sequence in the direction opposite to the direction of sequence transcription or read out.

“Polymerase chain reaction” (PCR) generally refers to a process that uses multiple cycles of nucleic acid denaturation, annealing of primer pairs to opposite strands (forward and reverse), and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. There are many permutations of PCR known to those of ordinary skill in the art.

“Position” refers to a particular amino acid or amino acids in a nucleic acid sequence.

“Primer” refers to an enzymatically extendable oligonucleotide, generally with a defined sequence that is designed to hybridize in an antiparallel manner with a complementary, primer-specific portion of a target nucleic acid. A primer can initiate the polymerization of nucleotides in a template-dependent manner to yield a nucleic acid that is complementary to the target nucleic acid when placed under suitable nucleic acid synthesis conditions (e.g. a primer annealed to a target can be extended in the presence of nucleotides and a DNA/RNA polymerase at a suitable temperature and pH). Suitable reaction conditions and reagents are known to those of ordinary skill in the art. A primer is typically single stranded for maximum efficiency in amplification, but may alternatively be double stranded. If double stranded, the primer is generally first treated to separate its strands before being used to prepare extension products. The primer generally is sufficiently long to prime the synthesis of extension products in the presence of the inducing agent (e.g. polymerase). Specific length and sequence will be dependent on the complexity of the required DNA or RNA targets, as well as on the conditions of primer use such as temperature and ionic strength. Preferably, the primer is about 5-100 nucleotides. Thus, a primer can be, e.g., 5, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95 or 100 nucleotides in length. A primer does not need to have 100% complementarity with its template for primer elongation to occur; primers with less than 100% complementarity can be sufficient for hybridization and polymerase elongation to occur. A primer can be labeled if desired. The label used on a primer can be any suitable label, and can be detected by, for example, spectroscopic, photochemical, biochemical, immunochemical, chemical, or other detection means. A labeled primer therefore refers to an oligomer that hybridizes specifically to a target sequence in a nucleic acid, or in an amplified nucleic acid, under conditions that promote hybridization to allow selective detection of the target sequence.

A primer nucleic acid can be labeled, if desired, by incorporating a label detectable by, e.g., spectroscopic, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or are otherwise known in the art. One of skill in the art will recognize that, in certain embodiments, primer nucleic acids can also be used as probe nucleic acids.

“Region” refers to a portion of a nucleic acid wherein said portion is smaller than the entire nucleic acid.

“Region of interest” refers to a specific sequence of a target nucleic acid that includes all codon positions having at least one single nucleotide substitution mutation associated with a genotype and/or subtype that are to be amplified and detected, and all marker positions that are to be amplified and detected, if any.

“RNA-dependent DNA polymerase” or “reverse transcriptase” (“RT”) refers to an enzyme that synthesizes a complementary DNA copy from an RNA template. All known reverse transcriptases also have the ability to make a complementary DNA copy from a DNA template; thus, they are both RNA- and DNA-dependent DNA polymerases. RTs may also have an RNAse H activity. A primer is required to initiate synthesis with both RNA and DNA templates.

“DNA-dependent DNA polymerase” is an enzyme that synthesizes a complementary DNA copy from a DNA template. Examples are DNA polymerase I from E. coli, bacteriophage T7 DNA polymerase, or DNA polymerases from bacteriophages T4, Phi-29, M2, or T5. DNA-dependent DNA polymerases may be the naturally occurring enzymes isolated from bacteria or bacteriophages or expressed recombinantly, or may be modified or “evolved” forms which have been engineered to possess certain desirable characteristics, e.g., thermostability, or the ability to recognize or synthesize a DNA strand from various modified templates. All known DNA-dependent DNA polymerases require a complementary primer to initiate synthesis. It is known that under suitable conditions a DNA-dependent DNA polymerase may synthesize a complementary DNA copy from an RNA template. RNA-dependent DNA polymerases typically also have DNA-dependent DNA polymerase activity.

“DNA-dependent RNA polymerase” or “transcriptase” is an enzyme that synthesizes multiple RNA copies from a double-stranded or partially double-stranded DNA molecule having a promoter sequence that is usually double-stranded. The RNA molecules (“transcripts”) are synthesized in the 5′-to-3′ direction beginning at a specific position just downstream of the promoter. Examples of transcriptases are the DNA-dependent RNA polymerase from E. coli and bacteriophages T7, T3, and SP6.

A “sequence” of a nucleic acid refers to the order and identity of nucleotides in the nucleic acid. A sequence is typically read in the 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997)″ “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

A “label” refers to a moiety attached (covalently or non-covalently), or capable of being attached, to a molecule, which moiety provides or is capable of providing information about the molecule (e.g., descriptive, identifying, etc. information about the molecule) or another molecule with which the labeled molecule interacts (e.g., hybridizes, etc.). Exemplary labels include fluorescent labels (including, e.g., quenchers or absorbers), weakly fluorescent labels, non-fluorescent labels, colorimetric labels, chemiluminescent labels, bioluminescent labels, radioactive labels, mass-modifying groups, antibodies, antigens, biotin, haptens, enzymes (including, e.g., peroxidase, phosphatase, etc.), and the like.

A “linker” refers to a chemical moiety that covalently or non-covalently attaches a compound or substituent group to another moiety, e.g., a nucleic acid, an oligonucleotide probe, a primer nucleic acid, an amplicon, a solid support, or the like. For example, linkers are optionally used to attach oligonucleotide probes to a solid support (e.g., in a linear or other logic probe array). To further illustrate, a linker optionally attaches a label (e.g., a fluorescent dye, a radioisotope, etc.) to an oligonucleotide probe, a primer nucleic acid, or the like. Linkers are typically at least bifunctional chemical moieties and in certain embodiments, they comprise cleavable attachments, which can be cleaved by, e.g., heat, an enzyme, a chemical agent, electromagnetic radiation, etc. to release materials or compounds from, e.g., a solid support. A careful choice of linker allows cleavage to be performed under appropriate conditions compatible with the stability of the compound and assay method. Generally a linker has no specific biological activity other than to, e.g., join chemical species together or to preserve some minimum distance or other spatial relationship between such species. However, the constituents of a linker may be selected to influence some property of the linked chemical species such as three-dimensional conformation, net charge, hydrophobicity, etc. Exemplary linkers include, e.g., oligopeptides, oligonucleotides, oligopolyamides, oligoethyleneglycerols, oligoacrylamides, alkyl chains, or the like. Additional description of linker molecules is provided in, e.g., Hermanson, Bioconjugate Techniques, Elsevier Science (1996), Lyttle et al. (1996) Nucleic Acids Res. 24(14):2793, Shchepino et al. (2001) Nucleosides, Nucleotides, & Nucleic Acids 20:369, Doronina et al (2001) Nucleosides, Nucleotides, & Nucleic Acids 20:1007, Trawick et al. (2001) Bioconjugate Chem. 12:900, Olejnik et al. (1998) Methods in Enzymology 291:135, and Pljevaljcic et al. (2003) J. Am. Chem. Soc. 125(12):3486, all of which are incorporated by reference.

“Fragment” refers to a piece of contiguous nucleic acid that contains fewer nucleotides than the complete nucleic acid.

“Hybridization,” “annealing,” “selectively bind,” or “selective binding” refers to the base-pairing interaction of one nucleic acid with another nucleic acid (typically an antiparallel nucleic acid) that results in formation of a duplex or other higher-ordered structure (i.e. a hybridization complex). The primary interaction between the antiparallel nucleic acid molecules is typically base specific, e.g., A/T and G/C. It is not a requirement to that two nucleic acids have 100% complementarity over their full length to achieve hybridization. Nucleic acids hybridize due to a variety of well characterized physio-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel (Ed.) Current Protocols in Molecular Biology, Volumes I, II, and III, 1997, which is incorporated by reference.

### EXAMPLES

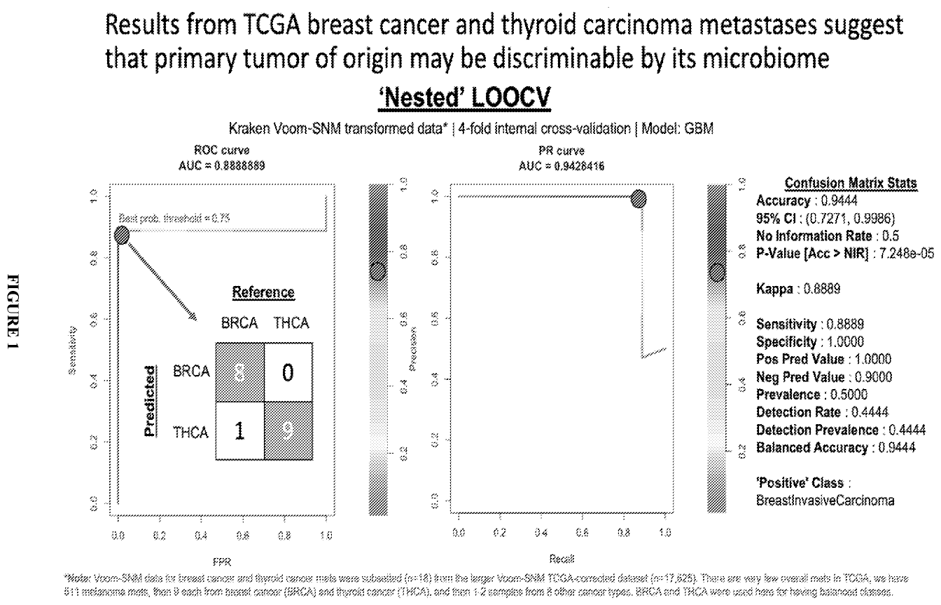

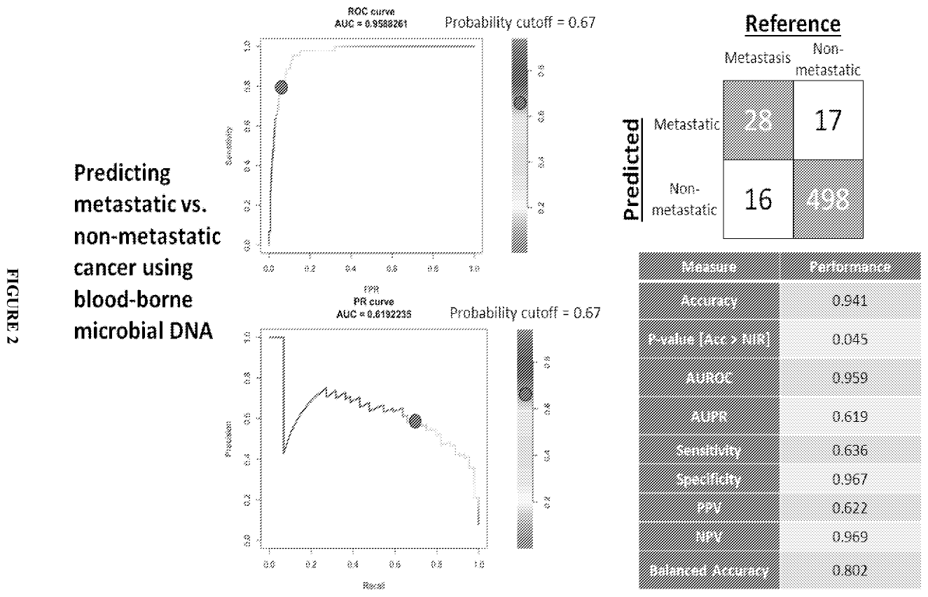

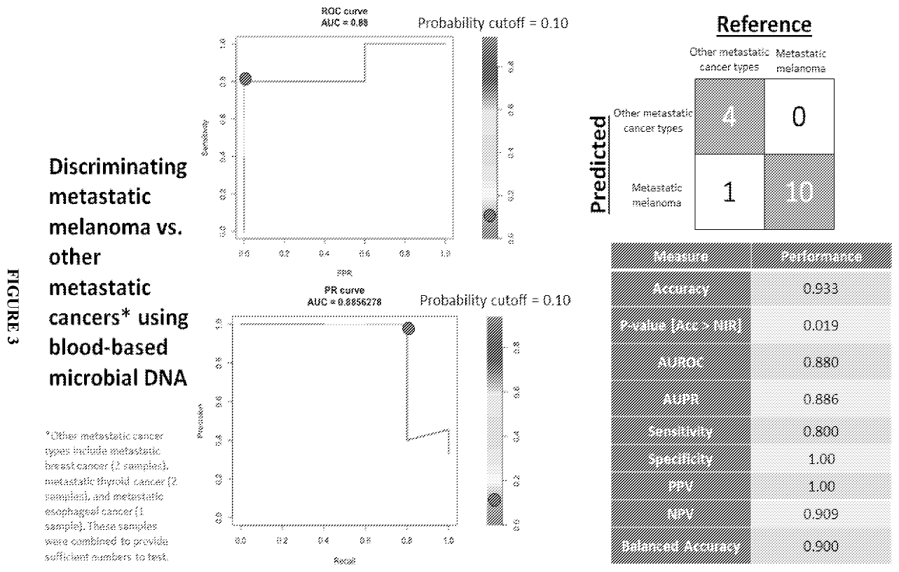

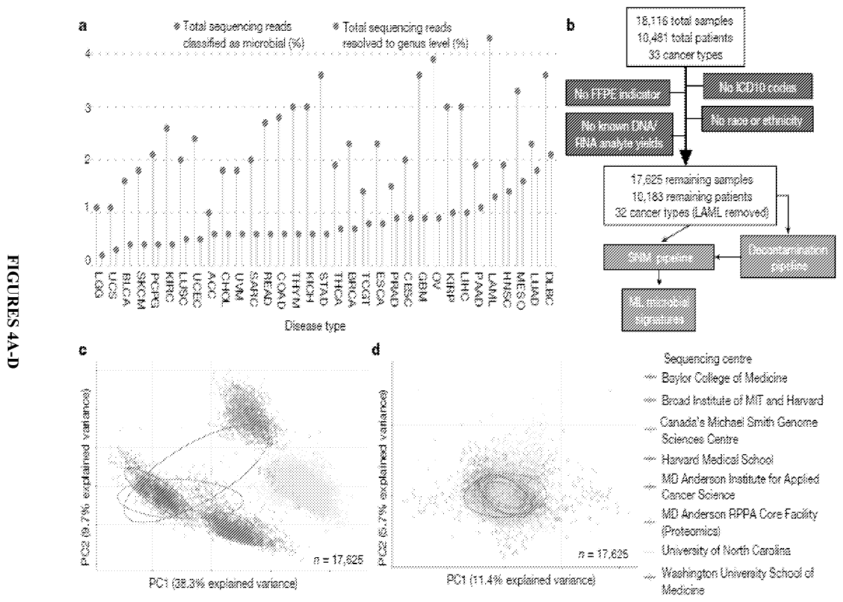

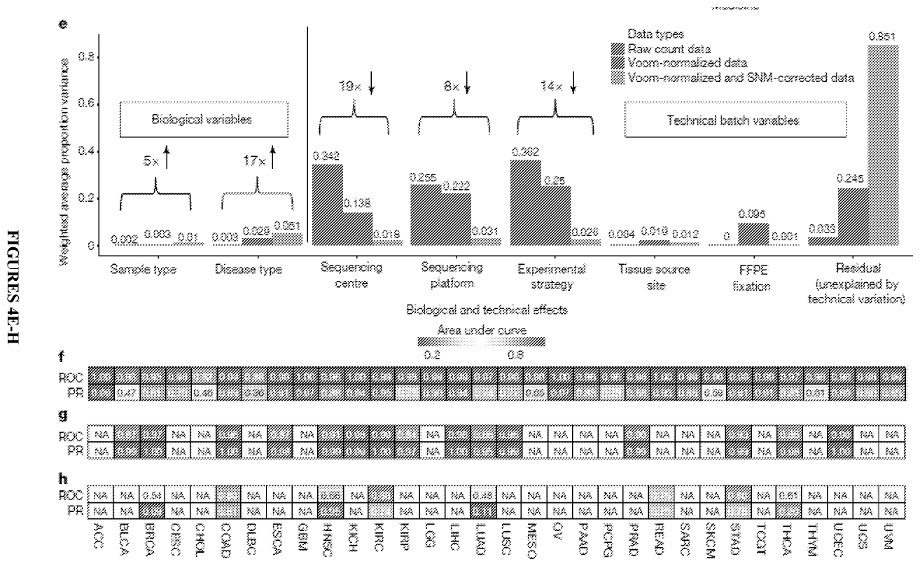

The present study was based on a preliminary analysis of more than 500 metastatic cancer tissue samples from 11 cancer types. FIG. 1 shows machine learning results that discriminate metastatic breast cancer and metastatic thyroid carcinoma by its tissue microbiomes, suggesting that primary tumor of origin may be discriminable by microbial features (since metastatic cancers are named on the basis of their tissue of origin). In at least one embodiment, Kraken Voom-SNM transformed data for breast cancer and thyroid cancer metastases were subsetted (n=18) from the larger TCGA Voom-SNM-corrected dataset (n=17,625). TCGA comprised 511 melanoma metastases, then 9 each from breast cancer (BRCA) and thyroid cancer (THCA), and then 1-2 samples from 8 other cancer types. BRCA and THCA were used here, as an illustrative example, for having balanced classes.

In at least one embodiment, a machine-learning model or algorithm as described herein is not required to determine microbial abundances; rather, that step is done prior using a taxonomy assignment algorithm. Then, in such embodiments, the machine-learning algorithm ranks importance of the microbes for determining which sample belongs to a certain cancer type. In various embodiments, Kraken is the taxonomy assignment algorithm (PMID: 24580807), and the machine learning algorithm is gradient boosting (Friedman, Jerome H. “Stochastic gradient boosting.” Computational statistics & data analysis 38.4 (2002): 367-378.), each of which are hereby incorporated by reference herein in its entirety.

Systematic characterization of cancer microbiome provides the opportunity to develop techniques that exploit non-human microorganism-derived molecules in the diagnosis of a major human disease. Following recent demonstrations that some types of cancer show substantial microbial contributions, whole-genome and whole-transcriptome sequencing studies in TCGA of 33 types of cancer from treatment-naive patients (a total of 18,116 samples) were re-examined for microbial reads, and unique microbial signatures in tissue and blood within and between major types of cancer were found using techniques described herein. These TCGA blood signatures remained predictive when applied to patients with stage Ia-IIc cancer and cancers lacking any genomic alterations currently measured on two commercial-grade cell-free tumor DNA platforms, despite the use of very stringent decontamination analyses that discarded up to 92.3% of total sequence data. In addition, using techniques described herein, could discriminate among samples from healthy, cancer-free individuals (n=69) and those from patients with multiple types of cancer (prostate, lung, and melanoma; 100 samples in total) solely using plasma-derived, cell-free microbial nucleic acids. This potential microbiome-based oncology diagnostic tool warrants further exploration.

Cancer is classically considered a disease of the human genome. However, recent studies have shown that the microbiome makes substantial contributions to some types of cancer. In particular, contributions of the fecal microbiome to gastrointestinal cancers. However, the extent and diagnostic implications of microbial contributions to different types of cancers remain unknown. The possibility of sample contamination during collection, processing, and sequencing limits these investigations, as procedural controls have rarely been implemented in cancer genomics projects. The use of recently developed tools to minimize the contributions of contaminants to microbial signatures may be utilized to enable the rational development of microbiome-based diagnostics, in various embodiments.

To characterize the cancer-associated microbiome, microbial reads from 18,116 samples across 10 k patients and 33 types of cancer from the TCGA compendium of whole-genome sequencing (WGS; n=4, 831) and whole transcriptome sequencing (RNA-seq; n=13,285) studies were examined. Other suitable datasets may be used and are contemplated within the scope of this disclosure. Microbial reads were previously identified in ad hoc analyses (including EBV in stomach adenocarcinoma and HPV in cervical cancer) and have been systematically studied in small subsets of samples (e.g., the viromes of 4,433 TCGA samples from 19 types of cancer and the bacteriomes of 1,880 TCGA samples across 9 types of cancer. Most TCGA sequencing data remain unexplored for microorganisms. As presented herein, comprehensive cancer microbiome data sets were created using two orthogonal microbial-detection pipelines, systematically measuring and mitigating technical variation and contamination. Machine-learning (ML) techniques were utilized to identify microbial signatures that discriminate among types and/or stages of cancer, and compare their performance.

A non-exhaustive list of cancer types and/or stages that may be identified using machine-learning models described herein include the following: Acute Myeloid Leukemia (LAML); Adrenocortical Carcinoma (ACC); Bladder Urothelial Carcinoma (BLCA); Brain Lower Grade Glioma (LGG); Breast Invasive Carcinoma (BRCA); Cervical Squamous Cell Carcinoma and Endocervical Adenocarcinoma (CESC); Cholangiocarcinoma (CHOL); Colon Adenocarcinoma (COAD); Lymphoid Neoplasm Diffuse Large B-cell Lymphoma (DLBC); Esophageal Carcinoma (ESCA); Glioblastoma Multiforme (GBM); Head and Neck Squamous Cell Carcinoma (HNSC); Kidney Chromophobe (KICH); Kidney Renal Clear Cell Carcinoma (KIRC); Kidney Renal Papillary Cell Carcinoma (KIRP); Liver Hepatocellular Carcinoma (LIHC); Lung Adenocarcinoma (LUAD); Lung Squamous Cell Carcinoma (LUSC); Mesothelioma (MESO); Ovarian Serous Cystadenocarcinoma (OV); Pancreatic Adenocarcinoma (PAAD); Pheochromocytoma and Paraganglioma (PCPG); Prostate Adenocarcinoma (PRAD); Rectum Adenocarcinoma (READ); Sarcoma (SARC); Skin Cutaneous Melanoma (SKCM); Stomach Adenocarcinoma (STAD); Testicular Germ Cell Tumors (TGCT); Thyroid Carcinoma (THCA); Thymoma (THYM); Uterine Carcinosarcoma (UCEC); Uterine Corpus Endometrial Carcinoma (UCS); Uveal Melanoma (UVM).

Because TCGA processing did not control for microbial contamination and excluded healthy individuals, an additional analysis was performed on blood, the TCGA sample type most likely to contain adventitious microbial contamination, using gold-standard microbiology protocols. Various embodiments focused on commensurably benchmarking signatures from plasma-derived microbial DNA against clinically available cell-free tumor DNA (ctDNA) assays. Deep metagenomic sequencing on plasma samples from individuals with prostate, lung, or skin cancers (n=100 total) and healthy, cancer, and HIV-free control participants (n=69) suggested that cell-free microbial profiles could be used to achieve healthy-versus cancer and caner-versus cancer discriminations. These findings suggest a new class of microbiome-based cancer diagnostic tools that may complement existing ctDNA assays for detecting and monitoring cancer.

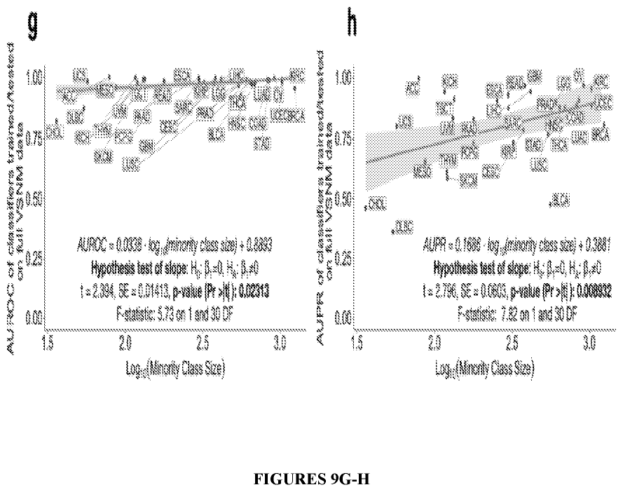

Using normalized data, stochastic gradient-boosting ML models were trained to discriminate between and within types and stages of cancer, according to various embodiments. The performance of these models was strong for discriminating (i) one cancer type versus all others (n=32 types of cancer) and (ii) tumor versus normal (n=15 types of cancer) (FIGS. 4f-g, FIGS. 9a-f; all performance metrics found at cancermicrobiome.ucsd.edu/CancerMicrobiome_DataBrowser). Differences in sensitivities and specificities between types of cancer may be partially due to differences in class sizes, as there was a significant linear relationship in one-cancer-type-versus-all-others comparisons between the minority class and AUROC (area under the receiver operating characteristics curve; P=0.0231) values (two-sided hypothesis test of slop; FIGS. 9g-h). Cancer microbial heterogeneity may also contribute to this differential performance. Tissue-based microbial models performed well for discriminating between stage I and stage IV tumors (n=8 types of cancer) for colon adenocarcinoma (COAD), STAD, and kidney renal clear cell carcinoma (KIRC), but not for the other five cancers tested (FIG. 4h), nor for discriminating intermediate stages (data not shown). These results suggest that microbial community structure dynamics may not correlate with cancer stages as defined by host tissue for all types of cancer.

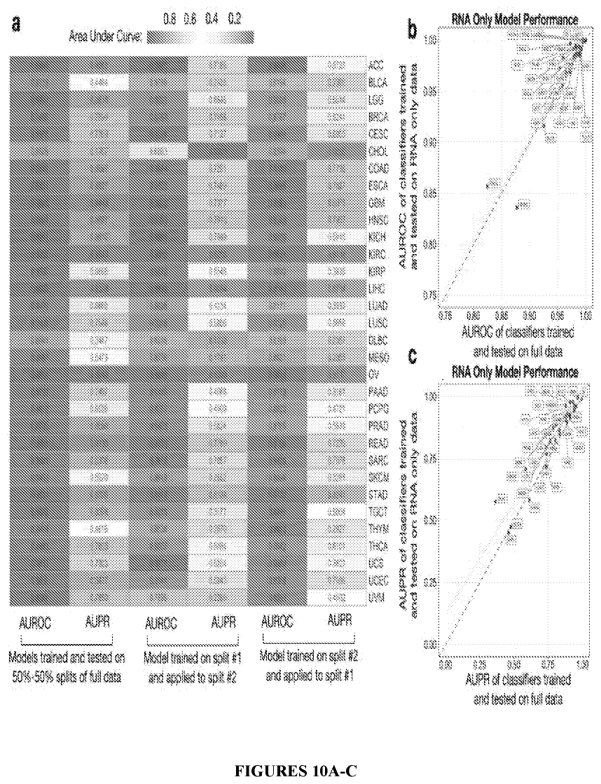

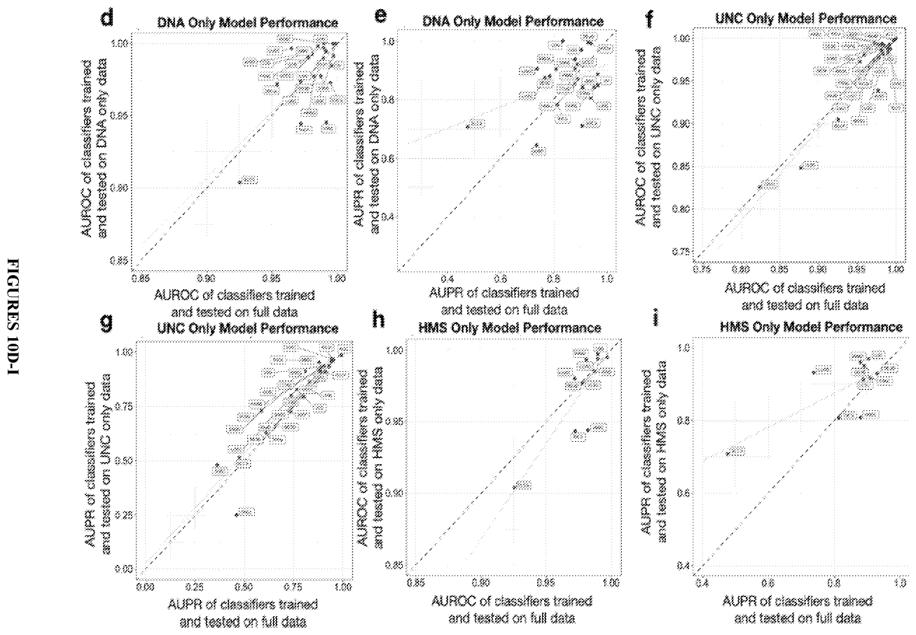

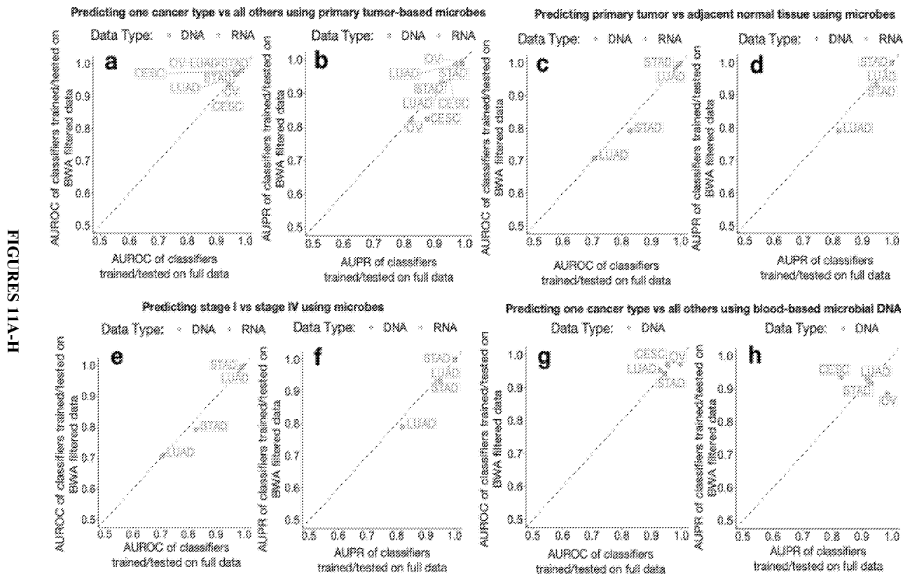

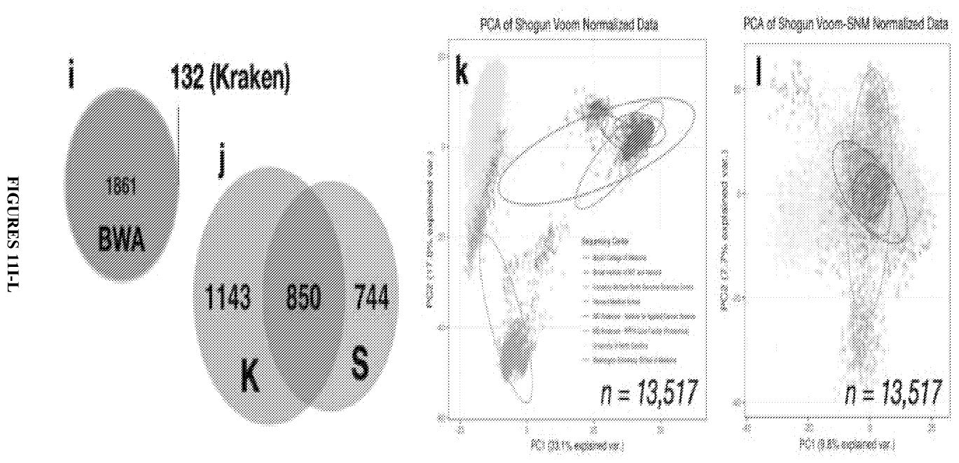

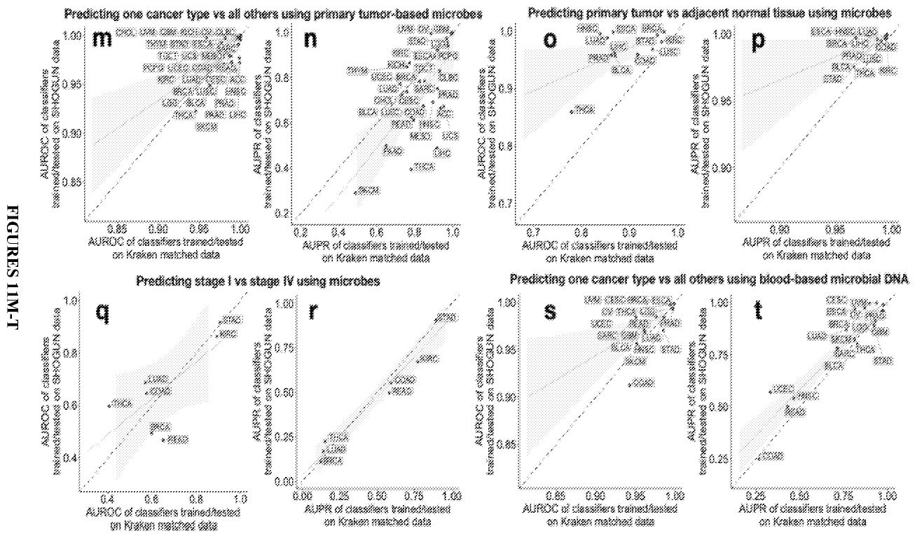

To evaluate the generalizability of such techniques across data sets, randomly raw TCGA microbial counts were sorted into two batches, all procedures on each independently were repeated, each independently trained model was tested on the other half of the data, and highly similar performance was found (FIG. 10a). Discriminatory microbial signatures held when examining singular methodologies (WGS or RNA-seq) or sequencing centers that performed either WGS or RNA-seq or using only genomic alignment-filtered Kraken data.

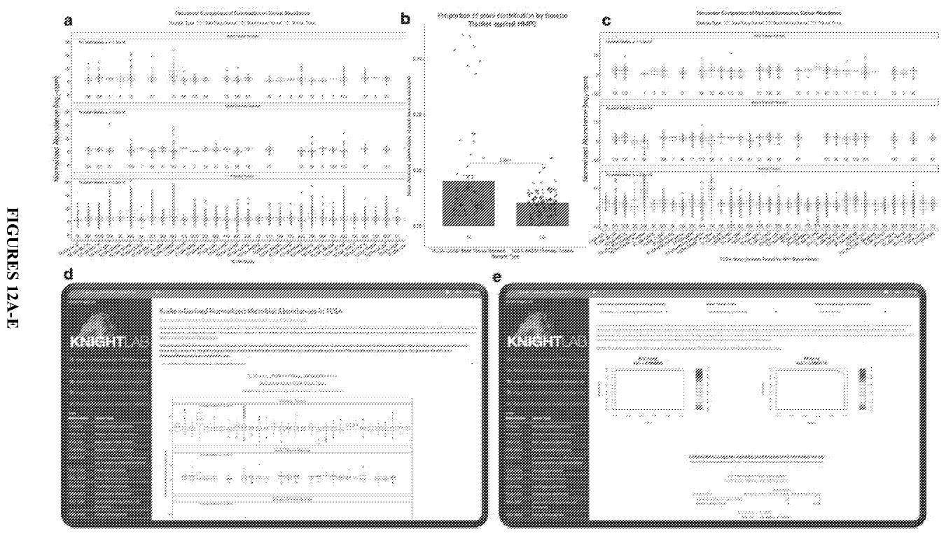

For further validation, SHOGUN was applied, an alignment-based microbial taxonomic pipeline using a reduced, phylogenetically based, bacteria-only database on 13,517 TCGA samples (WGS, n=3,434; RNA-seq, n=10,083) covering every analysis type of cancer (n=32), type of sample (n=7), sequencing platform (n=6), and sequencing center (n=8) in the Kraken-based analysis. The SHOGUN-derived data replicated the batch effects that have been identified in Kraken-derived data despite the use of a smaller, non-identical underlying database (FIGS. 11j-l). This data and a corresponding subset of Kraken-derived data (see Methods) was inputted independently into normalization and ML pipelines and found no major differences in discriminatory performance between the data sets (FIG. 11m-t). Together, the results imply that microbial communities are unique to each cancer type and that approaches of normalization and model training to distinguish cancers based on microbial profiles alone can be applied more broadly.

### Biological Relevance of Microorganism Profiles

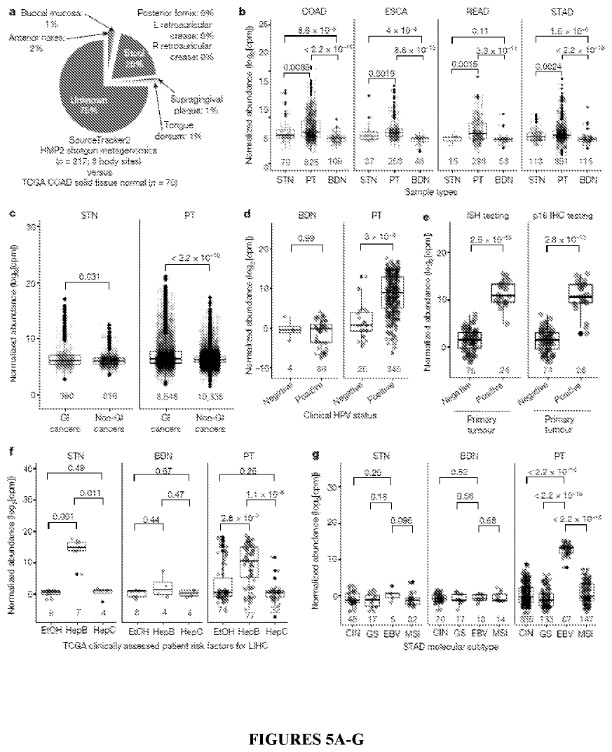

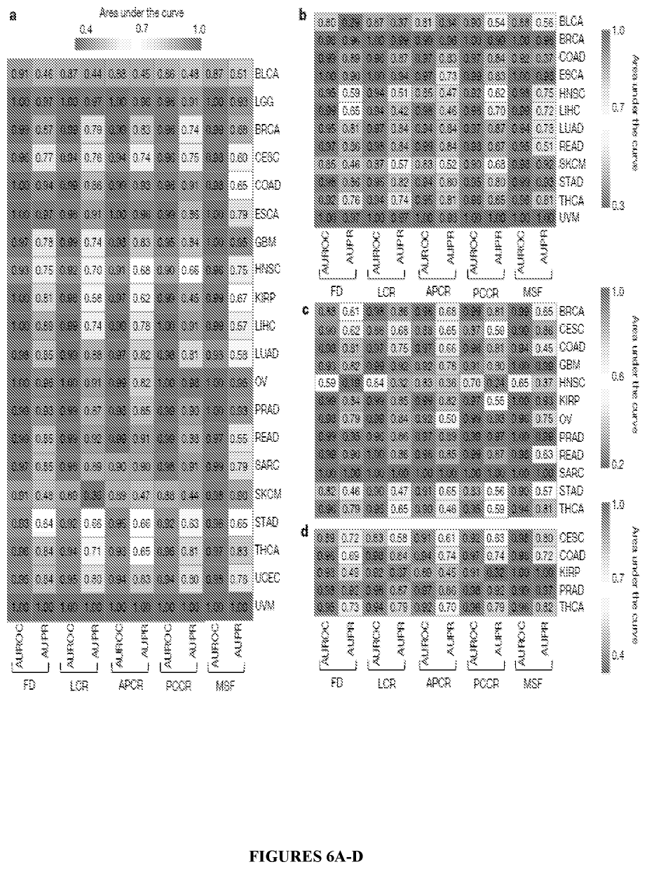

Given the strong discrimination of microbial signatures, evidence for their biological relevance using ecologically expected and/or clinically tested outcomes was examined. To assess whether cancer-associated microorganisms are ecologically expected (that is, part of the ‘native’ organ-specific commensal community), a Bayesian microbial-source tracking algorithm was trained on data from 217 samples across 8 body sites in the Human Microbiome Project 2 (HMP2) project that had been processed with microbial-detection and normalization pipelines described herein to estimate the body-site contribution from 70 solid-tissue normal samples in the COAD cohort and 122 skin cutaneous melanoma (SKCM) primary tumors (see Methods). Stool was the primary known body-site contributor only to COAD profiles (average mean±s.e.m. fractional contribution, 20.17±2.55%; FIG. 5a), but not SKCM profiles (one-tailed Mann-Whitney U-test, P=0.0014; FIG. 12b), suggesting that part of the community had a local source.

Fusobacterium spp. are important in the development and progression of gastrointestinal tumors and Fusobacterium was overabundant in primary tumors compared to solid-tissue normal samples (all P<8.5×10⁻³) and especially to blood-derived normal samples (all P<3.3×10⁻¹¹; FIG. 5b). Pan-cancer analyses also showed an overabundance of Fusobacterium when comparing all broadly defined gastrointestinal (GI) cancers in TCGA (n=8) against non-GI cancers (n=24) in both primary tumor tissue (P<2.2×10⁻¹⁶) and adjacent solid-tissue normal samples (P=0.031; FIG. 5c, FIG. 12a). Similar to previous investigations of STAD in TCGA, no differences were found in Helicobacter pylori between primary tumors and adjacent solid-tissue normal samples (P=0.72, data not shown; all tests were two-sided Mann-Whitney U-tests).

Clinically annotated TCGA viral infections were confirmed and microorganism-detection pipeline compared to studies that examined the TCGA virome using two different bioinformatic pipelines: (i) de novo metagenome assembly methods and (ii) read-based methods (PathSeq algorithm). There was differential abundance of the Alphapapillomavirus genus between primary tumors in individuals who were clinically tested as ‘positive’ or ‘negative’ for HPV infection in CESC and head and neck squamous cell carcinoma (HNSC) samples (all P<3×10⁻⁹, two-sided Mann-Whitney U-test; FIGS. 5d-e). Blood-derived normal samples from patients with CESC were used as negative controls and were not statistically different (P=0.99, two-sided Mann-Whitney U-test), and selective overabundance for Alphapapillomavirus held when comparing across all other types of cancer and sample types (FIG. 12c). Patients with liver hepatocellular carcinoma (LIHC) and a prior history of hepatitis B had selective overabundance of the HBV genus (Orthohepadnavirus) in both primary tumors and adjacent solid-tissue normal samples compared to patients with LIHC and a prior history of alcohol consumption and hepatitis C (Hepacivirus genus) (FIG. 5f; primary tumor P≤2.8×10⁻⁷; solid-tissue normal P<0.011); blood-derived normal samples were used as negative controls and were not statistically different (P≥0.44; all tests were two-sided Mann-Whitney U-tests). Also consistent with the previous reports, the genus for EBV (Lymphocryptovirus) was selectively overabundant in EBV-infected primary tumors compared to patients assigned to other STAD molecular subtypes (FIG. 5g; P≤2.2×10⁻¹⁶). Solid-tissue normal and blood-derived normal samples were used as negative controls and were not statistically different (blood, P≥0.52; tissue, P≥0.096; all tests were two-sided Mann-Whitney U-tests).

These data are consistent with information about feature importance provided by models in one-cancer-type-versus-all-others distinctions. Namely, cancers with known microbial ‘drivers’ or ‘commensals’ provided initial evidence that the models were ecologically relevant; for example, Alphapapillomavirus genus was the most important feature for identifying CESC tumors; for COAD tumors, the Faecalibacterium genus; for LIHC tumors, the Orthohepadnavirus genus was the second most important feature (after the hepatotoxic Microcystis genus). Collectively, the findings provide ecological validation of bioinformatic and normalization approaches for viral and bacterial data while extending the results to many more samples and microorganisms.

### Measuring and Mitigating Contamination

In various embodiments, it may be important to measure and mitigate the potential effects of contamination, in order to best characterize putative cancer-associated microorganisms. Previous work identified just six contaminants in TCGA (Staphylococcus epidermidis, Propionibacterium acnes, Ralstonia spp., Mycobacterium, Pseudomonas, and Acinetobacter) based on common low-read abundances across types of cancer, but recent studies have shown that external contaminants more consistently have frequencies that are inversely correlated with sample analyte concentration and can be detected using a robust statistical framework.

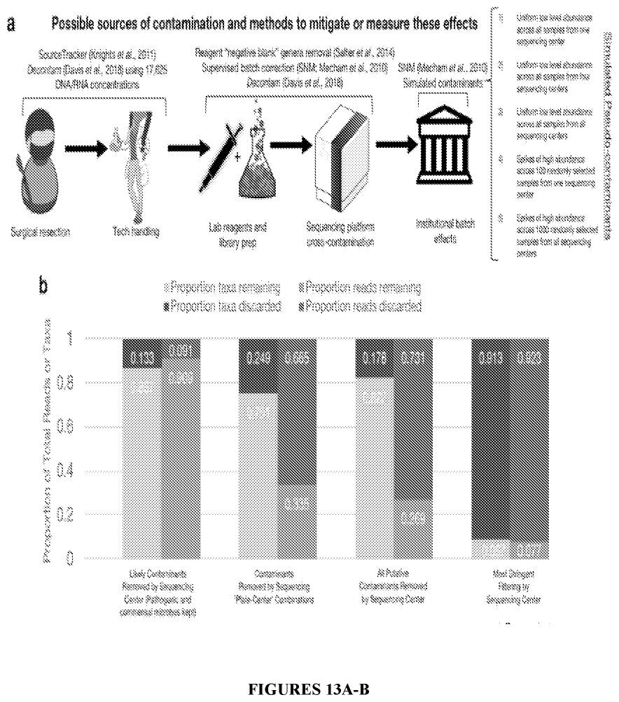

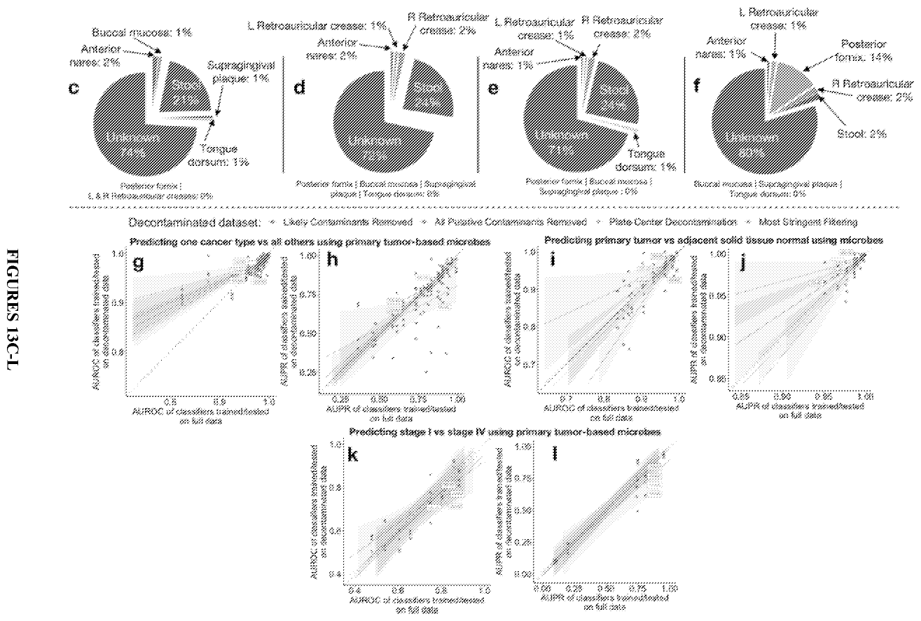

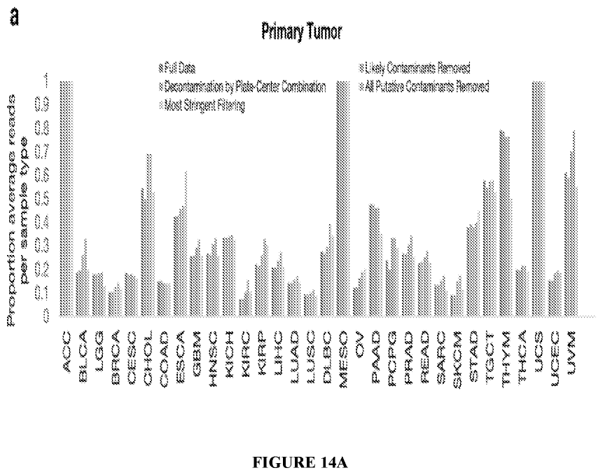

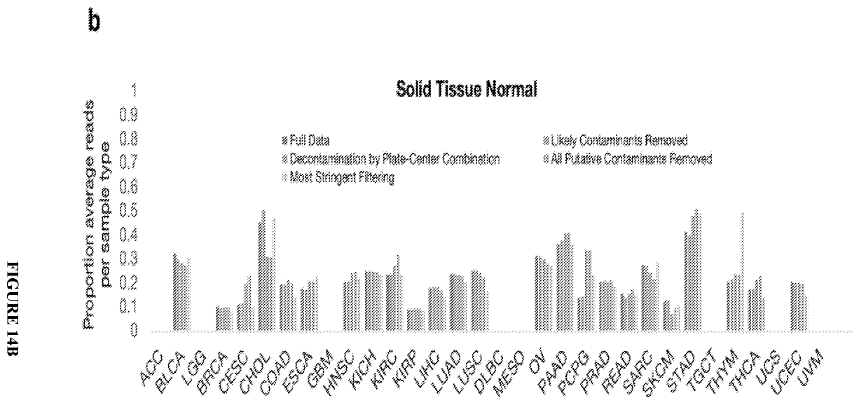

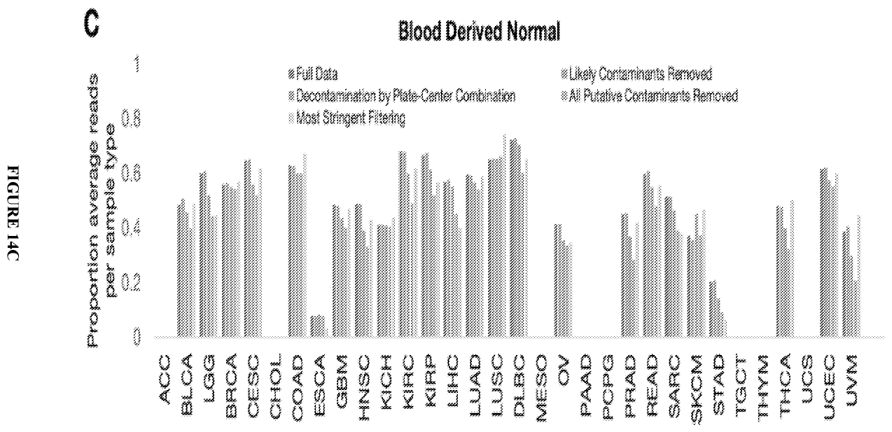

Based on the latter approach, DNA and RNA concentrations calculated during TCGA sample processing (n=17,625) and taxon read fractions (n=1,993) were used to identify putative contaminants, and also removed genera typically found in ‘negative blank’ reagents (n=94 genera; see Methods). FIG. 13a outlines the approaches taken from surgical resection to bioinformatic processing; five types of pseudo-contaminants were spiked into the raw data set to track through decontamination, supervised normalization, and ML. Given known technical variation (FIGS. 4c-e), samples were processed in batches by sequencing center (n=8) and removed taxa found to be a contaminant at any center. This identified 283 putative contaminants, including 19.1% (n=18 genera) of the reagent ‘blacklist’. After combining these two lists (n=377 genera), the literature was manually reviewed to re-allow pathobiont genera or mixed-evidence genera (both a pathogen and common contaminant; for example, Mycobacterium). This resulted in two data sets, one with likely contaminants removed and another with all putative contaminants removed. A third ‘most stringent filtering’ data set was created, that discarded about 92% of the total reads using a stricter filtering schema (see Methods; FIG. 13b). Finally, samples were grouped into individual sequencing plates at each center and removed all putative contaminants identified in any one ‘plate-center’ batch (n=351; see Methods), in addition to the aforementioned reagent blacklist (497 genera in total). Decontamination did not appear to differentially affect the types of sample or cancer under study (FIGS. 14a-c).

In at least some embodiments, in silico decontamination methods are not substitutes for implementing gold-standard microbiology practices on cancer samples, including sterile processing, sterile-certified reagents, negative blanks of reagents processed from start to finish, and multiple-sample pooling as ‘positive’ controls. The in silico tools described here reflect the state of the art, but are not designed to detect abundant ‘spikes’ of contaminants or cross-contaminants. These latter contaminants should not drive uniform discriminatory signals between and within types of cancer collected over many centers and years, but may limit biological conclusions, particularly in small studies, if not controlled.

In at least some embodiments, a risk with stringent decontamination is that real signals that reflect commensal, tissue-specific microbial communities and concomitant cancer-predictive microbial profiles may be discarded. To evaluate this concern, the body-site attribution percentages may be re-calculated for COAD solid-tissue normal samples (n=70), and found that successively stringent decontamination improved recognition of concomitant tissues before they became unrecognizable (FIGS. 13c-f).

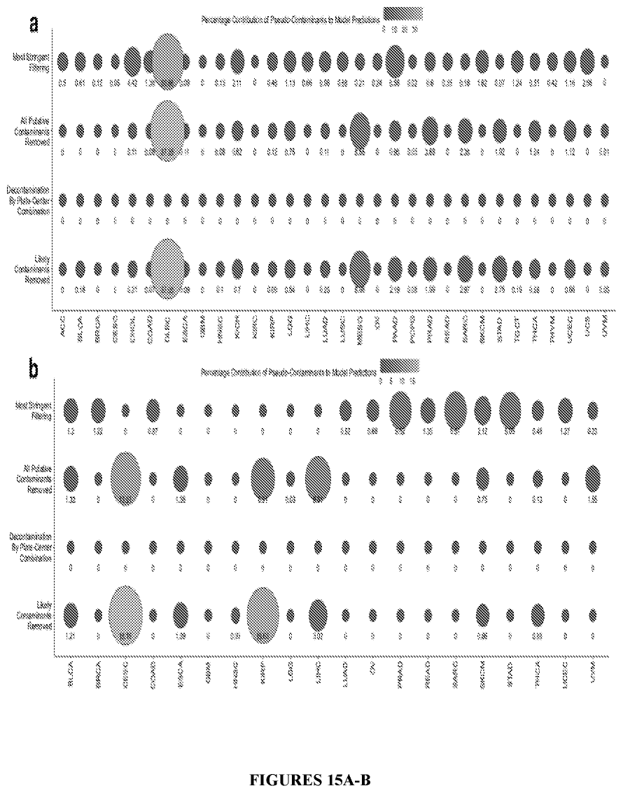

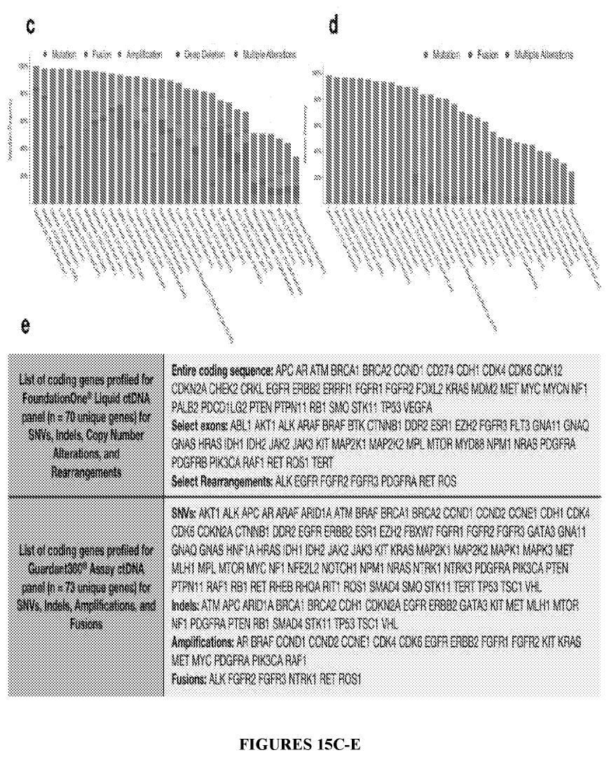

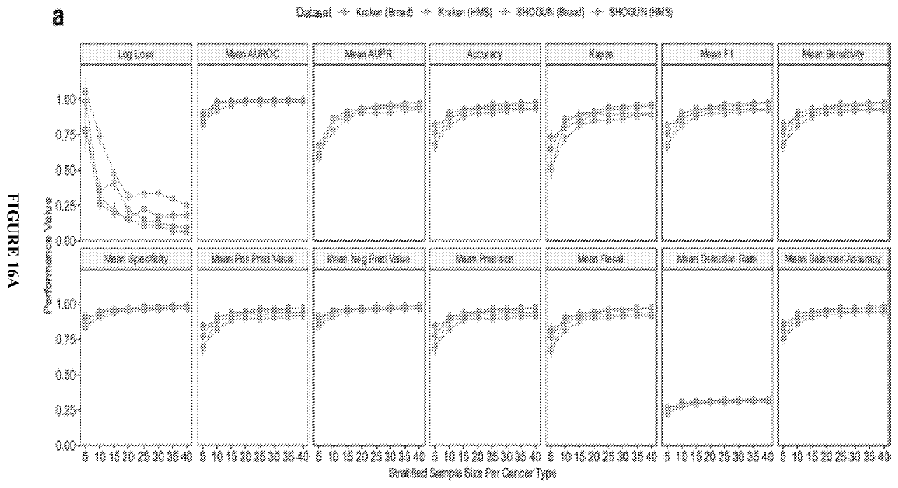

ML models shown in FIG. 4f-h were recalculated and compared their performances before and after each decontamination approach (FIGS. 13g-l). Most models did not rely on spiked pseudocontaminants (FIG. 15a), although the lymphoid neoplasm diffuse large B cell lymphoma (DLBC) and mesothelioma (MESO) models (with very few available samples) appear to be exceptions and may be unreliable. As expected, comparisons where knowledge about the tissue type is informative (for example, COAD versus all other cancer types) generally performed less well with stringent decontamination, but within-tissue comparisons (for example, tumor versus normal) often performed equally well or better. These results suggest that stringent filtering may be desirable in certain comparisons, but a universal approach to decontamination may preclude biologically informative results.

### Predictions Using Microbial DNA in Blood

There is mounting evidence that blood-based microbial DNA (mbDNA) can be clinically informative in cancer, including those featuring blood-barrier or lymphatic disruptions (for example, COAD), but it is unclear how broadly this applies based on the current state of the art. Using WGS data from TCGA blood samples, ML strategies were applied to the full data set and four decontaminated data sets and found that blood-borne mbDNA could discriminate between numerous types of cancer (FIG. 6a), regardless of the microbial taxonomic algorithm and database used for classification or when using only genomic-alignment-filtered Kraken data (FIG. 11g, FIG. 11h, FIG. 11s, and FIG. 11t). Retrospective analysis showed that few models included spiked pseudo-contaminants for predictions (FIG. 15b); models that did (CESC, KIRP, LIHC) may be less trustworthy.

Spurred by these findings, ML models were benchmarked against existing ctDNA assays, focusing on circumstances under which ctDNA assays fail: stage Ia-IIc cancers and tumors without detectable genomic alterations. After removal of all blood-derived normal samples from patients harboring stage III or IV cancers, new ML models were built and found that they were able to discriminate well between types of cancer using blood mbDNA (FIG. 6b). Gene lists from the Guardant360 and FoundationOne Liquid assays were further used to filter out TCGA patients with one or more targeted modifications (about 70%; FIGS. 15c-e) and found that the same ML approach showed good discrimination for most remaining cancer types (FIGS. 6c-d).

These analyses are limited by the fact that ctDNA assays use plasma rather than whole blood, and that the distribution of mbDNA among blood compartments is unknown. It is impossible to tell whether mbDNA came from live or dead microorganisms, as RNA data were unavailable, or whether mbDNA is cell-free in host leukocytes, as TCGA standard operating procedures (SOPs) allow whole bloody or buffy coat extraction (see Methods). It is also impossible to know the origin of blood mbDNA without examining primary specimens and, possibly, matched gut epithelia, as certain types of cancer may leak’ mbDNA in unexpected ways (e.g., gut bacterial translation in leukemia). There is likely to be a continuum of ideal decontamination, as the effect of decontamination on model performance varied across types of cancer, but filtering was limited by (i) not having access to the primary specimens, (ii) genus-level taxonomic resolution, and (iii) not knowing which non-TCGA samples were concurrently processed.

### Validating Microbial Signatures in Blood

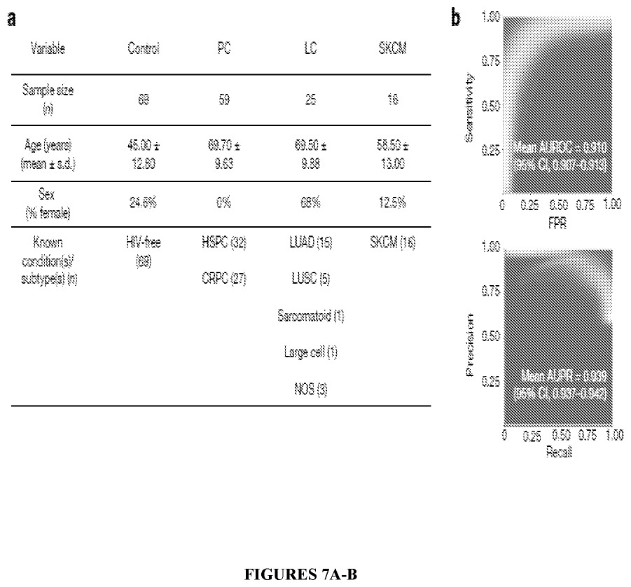

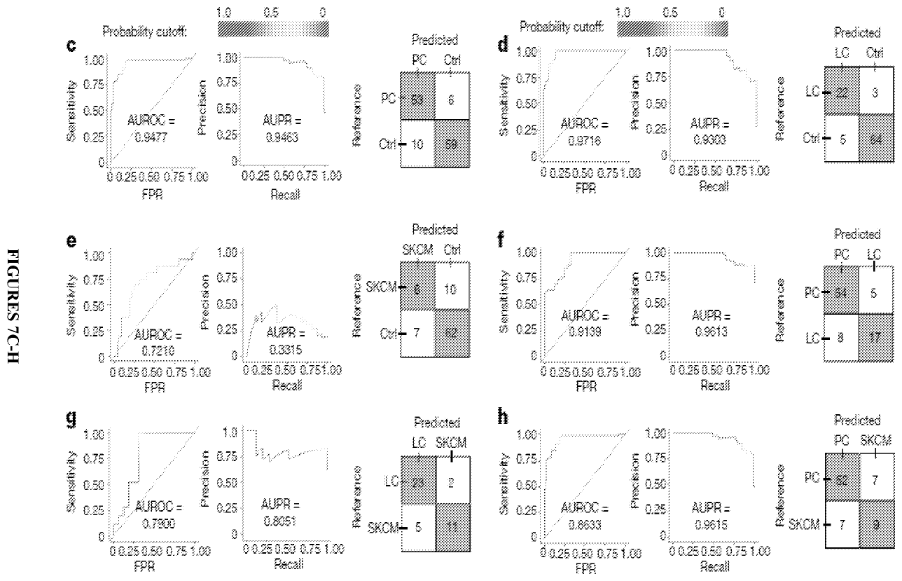

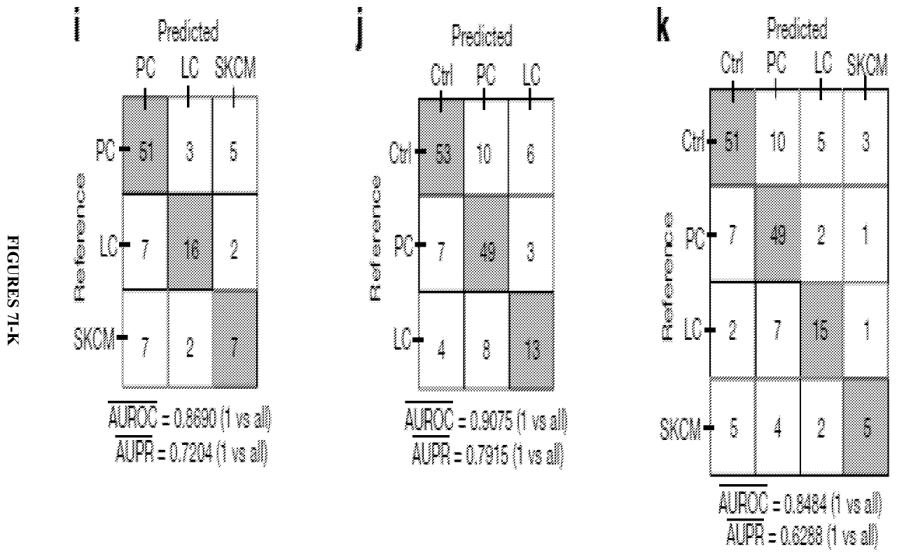

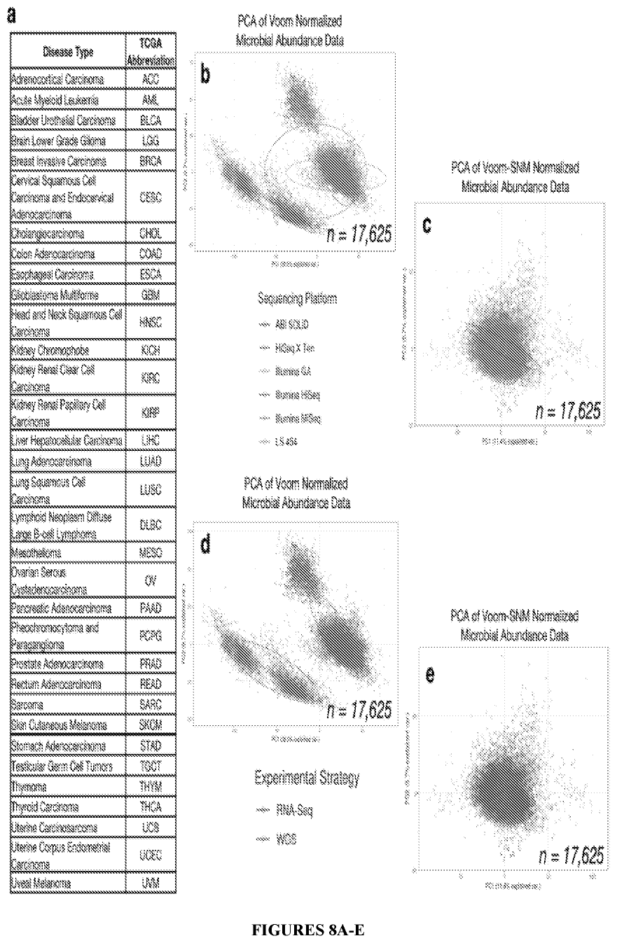

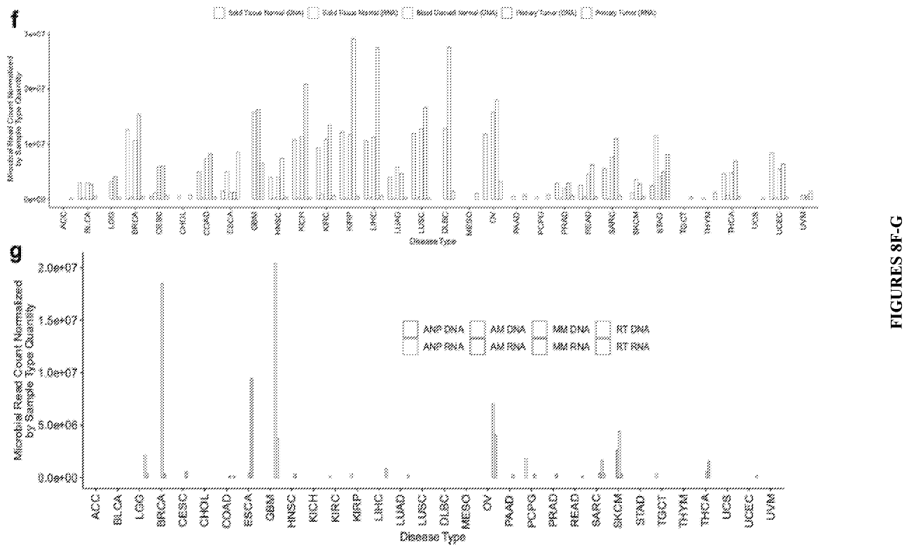

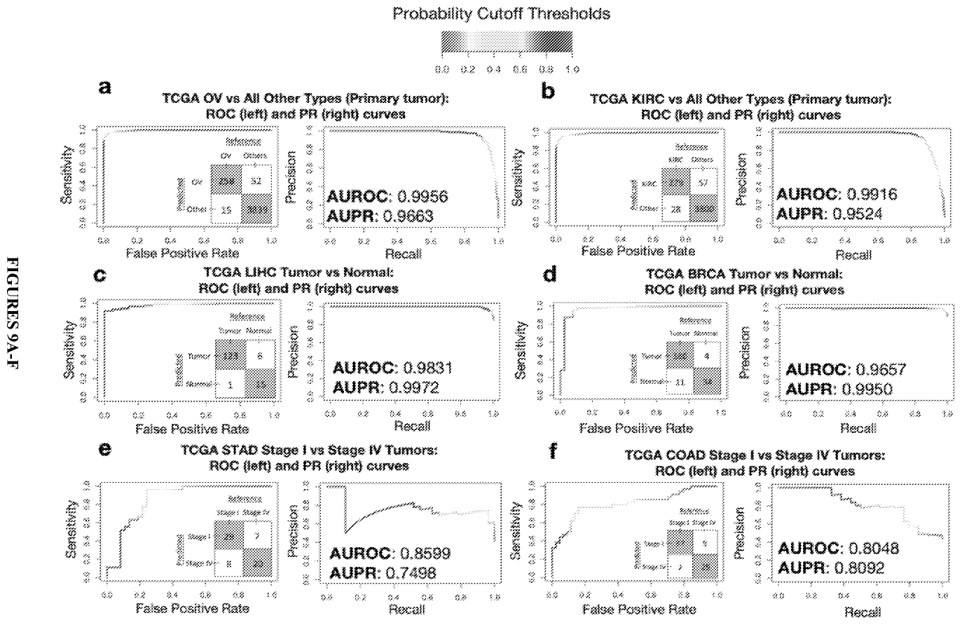

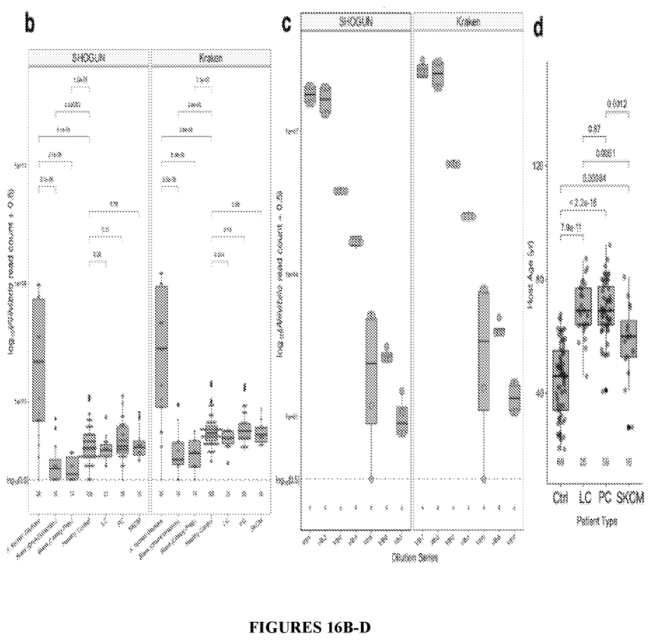

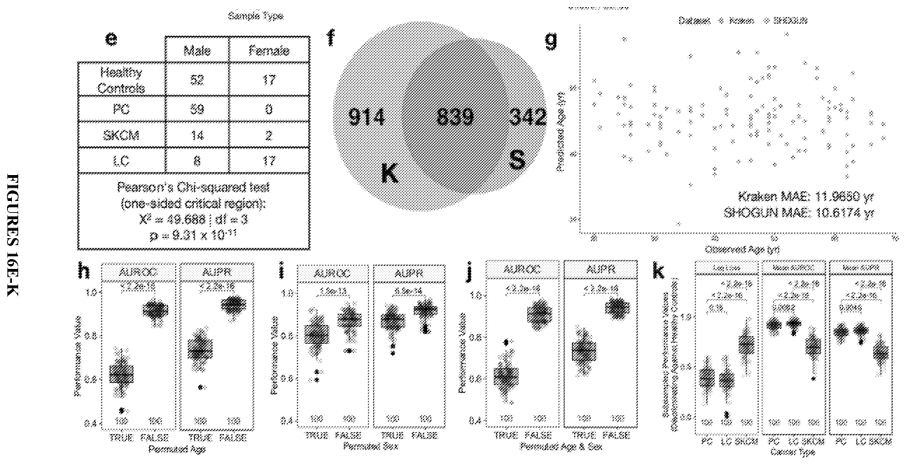

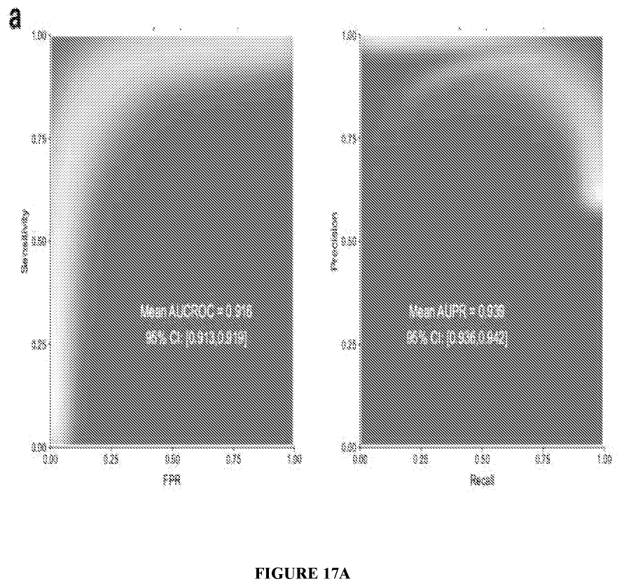

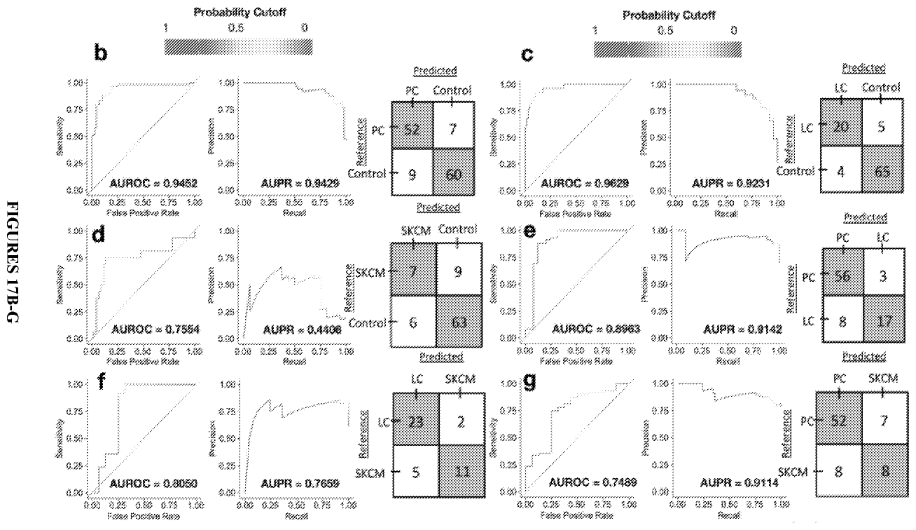

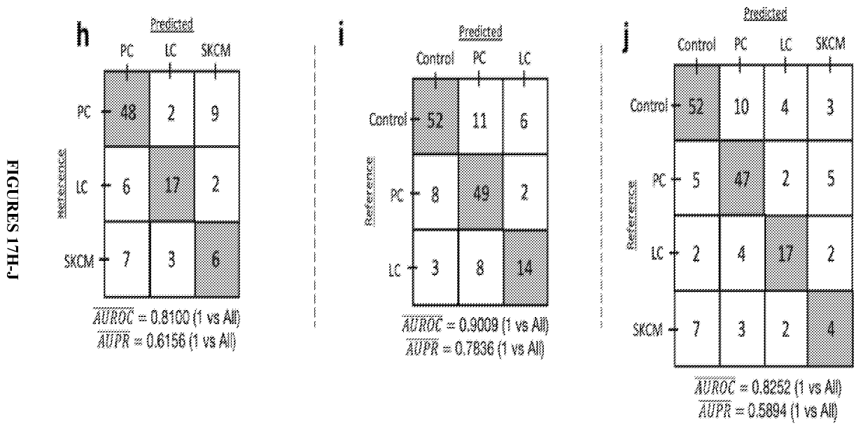

To demonstrate the real-world utility of these results while benchmarking against plasma-based ctDNA assays, the use of plasma-derived, cell-free mbDNA signatures to discriminate among healthy individuals and multiple types of cancer was used in a validation study while implementing gold-standard microbiology controls for low biomass studies. Although plasma represents a distinct subset of whole blood that is not studied in TCGA, limiting direct comparability, it carries major advantages in archival stability (for example, freezability), biorepository availability, and biological interpretation (that is, non-living material). The cohort included 69 cancer- and HIV-free individuals and 100 patients with one of three types of high-grade (stage III-IV) cancer: prostate cancer (n=59; PC); lung cancer (n=25; LC), and melanoma (n=16; SKCM) (FIG. 7a). Without prior literature to estimate effect sizes, independent simulations on TCGA blood samples from matched types of cancer at The Broad Institute and HMS to estimate minimum sample sizes (FIG. 16; see Methods). Cell-free DNA was extracted from these plasma samples with extensive controls (FIGS. 16b-c), and processed for whole metagenomic sequencing by a limited set of users, using a single library preparation method, in a single batch, in one deep-sequencing run. In various embodiments, techniques involved performing human-read removal, classification of remaining reads by Kraken, stringent decontamination using both DNA concentrations and negative blanks, and Voom-SNM. Demographic comparisons and permutation analyses suggested necessary normalization for age and sex (FIGS. 16d-e, h-j; see Methods), and direct age regression performance showed mean absolute errors similar to the gut microbiome (FIG. 16g). ‘Bootstrapping’ the same ML protocol used in the TCGA analyses showed strong, generalizable discrimination between healthy control individuals and grouped patients with cancer (FIG. 7b; see Methods). Because of the small sample sizes being used, leave-one-out (LOO) iterative ML was performed on the normalized data and found high discriminatory performance in pairwise and multiclass comparisons between and among healthy samples and types of cancer except for the smallest SKCM cohort (FIGS. 7c-k). Therefore, iteratively subsampled PC and LC groups to match the SKCM cohort size and performed pairwise LOO discrimination of each type of cancer against subsampled healthy controls (FIG. 16k; see Methods). The PC and LC cohorts were still separable at the same cohort size as SKCM (mean (95% confidence interval (CI)) AUROC=0.891 (0.879-0.903); mean (95% CI) AUPR=0.827 (0.815-0.839); 100 iterations), revealing universal deficits in SKCM performance. This deficit may have a biological basis, as SKCM was the second-worst performer in TCGA blood discriminations for four of five data sets tested (FIG. 6a), although this warrants further confirmation. To ensure that the microbial assignments by Kraken were valid, all bioinformatic, normalization, and ML steps using bacterial assignments from SHOGUN and its separate database were repeated, which showed highly concordant performances (FIG. 17). Refinements of the taxonomic assignments for cfDNA signatures are contemplated as microbial databases improve. The plasma microbial abundances detected can be explored at cancermicrobiome.ucsd.edu/CancerMicrobiome_DataBrowser (FIGS. 12d-e).

Collectively, the data suggest that there are widespread associations between diverse types of cancer and specific microbiota. These microbial profiles appear to discriminate within and between most types of cancer, including when using blood-based mbDNA at low-grade tumor stages and in patients without any detectable genomic alterations on commercial ctDNA assays. These results often remain valid even after extensive internal validation checks and decontamination, which at times discards more than 90% of the total data. The high discriminatory performance among healthy control individuals and patients with multiple types of cancer using only cell-free mbDNA in plasma, while adopting more extensive internal and external contamination controls than TCGA, suggests that clinically relevant and retrospective testing using widely available samples are feasible and generalizable. Nonetheless, results suggest that a new class of microbiome-based cancer diagnostic tools can provide substantial future value to patients.

In comparison to prior art methods that rely on human information to diagnose a metastasis's tissue of origin, with 69-79% accuracy (PMID: 23287002), the present invention provides at least about 94% accuracy based on microbial information. It is envisaged that this accuracy can be raised even further, such as 95% to 100% accuracy, by combining microbial information with host information. The accuracy was determined using the dataset previously published by inventor (PMID: 32214244), in which it was explored whether metastatic cancer types could be separated on the basis of their intratumoral or blood-derived microbiota. Since this dataset had samples from several known metastatic cancer types (e.g., breast cancer, thyroid cancer, melanoma) that had been harvested for microbial DNA and RNA, machine learning was employed to characterize the performance of distinguishing cancer type solely using microbial DNA and RNA.

The machine learning methods described herein had been developed for and previously published by the inventor (PMID: 32214244), as well as published in PCT application WO2020093040A1 (each incorporated by reference herein in its entirety). As an example, breast cancer metastases were compared to thyroid cancer metastases solely using intratumoral microbial nucleic acids and showed high discriminatory performance (area under ROC curve=0.889, area under PR curve=0.943, accuracy=94.4%). Effectively, one embodiment of the invention provides diagnosis of a tissue of origin for metastatic cancers using microbial information. In other embodiments, the invention provides analyses to distinguish hosts with primary tumors from hosts with metastatic tumors, thereby diagnosing the presence of a metastasis.

### Methods

### TCGA Data Accession

All TCGA sequence data were accessed via the Cancer Genomics Cloud (CGC) as sponsored by SevenBridges. SOPs for TCGA were accessed via the NCI Biospecimen Research Database. Matched patient metadata, including molecular subtypes, were accessed via the CGC through both SevenBridges and the Institute for Systems Biology (ISB), via the TCGA-Mutations R package, or were taken directly from the supplementary data of the respective TCGA publications. Genomic alteration statuses for all TCGA patients were queried and downloaded via cBioPortal. Gene panels for commercial ctDNA assays were access from company white papers for the Guardant360 assay and the FoundationOne Liquid assay. For TCGA metadata accession and transformation from hierarchical formats to flat tables, the SevenBridges's metadata ontology was queried and organize the data where possible; for information not stored in that ontology, the ISB CGC R programming language API was used to access its recent metadata release.

Bioinformatic tools were either loaded directly from the CGC platform (for example, samtools, BWA) or uploaded and run as separate Docker containers in order to create customized workflows. These workflows take sample BAM files as inputs and label which DNA or RNA reads within each sample are microbial.

Sequence reads that did not align to known human reference genomes (based on mapping information in the raw BAM files) were mapped against all known bacterial, archaeal, and viral microbial genomes using the Kraken algorithm. A total of 71,782 microbial genomes were downloaded using RepoPhlan, of which 5,503 were viral and 66,279 were bacterial or archaeal. On the basis of prior literature, bacterial and archaeal genomes were filtered for quality scores of 0.8 or better, which left 54,471 of them for subsequent analysis, or a total of 59,974 microbial genomes.

As previously described, the Kraken algorithm breaks each sequencing read into k-mers (default 31-mers, for example) and exactly matches each k-mer against a database of microbial k-mers, which was built from the 59,974 microbial genomes described above before running the algorithm. The set of exact k-mer matches for a given read, in turn, provides a putative taxonomy assignment of the lowest common ancestor for that read, most accurately to the genus level, to which is summarized in the data presented herein. The matching and classification operations are orders of magnitude faster than performing direct genome alignments. As a safeguard against false positives and to properly benchmark the pipeline, four types of cancer (STAD, CESC, OV and LUAD) were selected and aligned the reads Kraken classified as microbial to the 59,974 microbial genomes using BWA, which is computationally more expensive but yields a result with higher specificity and taxonomic resolution (that is, to species and strain level). The four types of cancer that were directly aligned included CESC as a putative positive viral control (for HPV), STAD as a putative positive bacterial control (for H. pylori), and two others (LUAD, OV) based on microbial signatures in the literature and/or available mass-spectrometry proteomic information (data not shown). It was determined that 98.91% of reads that were classified to genus level or lower by Kraken (on which various findings are based) also aligned with BWA to the microbial data (bacteria, archaea, viruses), or a false positive rate of 1.09%, suggesting that the genus-level, Kraken-labelled, pan-cancer microbial reads were sufficiently usable for future analyses.

### SHOGUN TCGA Bioinformatic Processing

To evaluate the robustness of cancer type discriminations using different taxonomic identification algorithms, a previously published shallow shotgun taxonomic assignment approach was utilized and a separate, phylogeny-centric database called Web of Life (WoL; PMID: 31792218; n=10,575 bacterial and archaeal genomes) on TCGA samples. SHOGUN utilizes computationally intensive direct genomic alignments for taxonomy assignments rather than an ultrafast k-mer-based approach like that used by Kraken. To reduce processing time for TCGA samples, reads classified as microbial in origin by Kraken were used as input for the SHOGUN alignment function, which used Bowtie2 to map reads against the WoL database to generate taxonomy profiles. In total, 13,517 samples (WGS: n=3,434; RNA-seq: n=10,083) were processed, covering all TCGA types of cancer (n=32), sample types (n=7), sequence centers (n=8) and sequencing platforms (n=6) under study in the Kraken analysis, including 21 TCGA types of cancer (n=9,444 sample) that had all samples in the Kraken analysis re-analyzed by SHOGUN. Profiles were then collapsed to the genus level using QIIME 2. Analyses were run on a local compute cluster that comprised 1,024 Intel Ivy-bridge compute cores, as well as 384 AMD compute cores, and 12 TB of total RAM for approximately 5 months of computational wall-time. Typical job submissions for a single cancer type used were −30 cores and −250 GB of RAM.

### Quantitative Measurement and Normalization of TCGA Technical Variation

Cognizant of how technical variations between TCGA sequencing centers (n=8), sequencing platforms (n=6), experimental strategy (WGS vs. RNA-seq), and possible contamination could confound the results, a pipeline was developed to quantify and remove batch effect while maintaining or increasing the signal attributed to biological variables. In brief, samples with poor metadata quality were filtered out (that is, missing race or ethnicity, ICD10 codes, DNA/RNA analyte amounts, or FFPE status information); transformed the discrete taxonomical count data to approximately normally distributed, log-count per million (log-cpm) data using the Voom algorithm, which models and removes the data's heteroscedasticity; and lastly, performed supervised normalization (SNM) on the data to remove all significant batch effects while preserving biological effects. Voom is traditionally used in combination with limma for differential expression (or abundance) analysis of discrete count data, but was used for the algorithmic transformation to ‘microarray-like’ data, which permitted subsequent SNM. The Voom and SNM model matrices were equivalent and built using sample type as the target biological variable (n=7; for example, primary tumor tissue) owing to expected biological differences between them, for which signal should be preserved using the SNM; conversely, the following were modeled as technical covariates to be mitigated during SNM: sequencing center (n=8), sequencing platform (n=6), experimental strategy (n=2), tissue source site (n=191), and FFPE status (n=2; yes or no). It was not possible to model disease type as the target biological variable owing to complete confounding between certain types of cancer and sequencing centers (that is, some types of cancer were only sequencing at one TCGA site). During the Voom transformation, weighted trimmed mean of M-values (TMM) normalization from the edgeR package was used for most data (‘full dataset’, ‘likely contaminants removed’ data, ‘plate-center decontaminated’ data, and ‘all putative contaminants removed’ data) while dropping unvarying features (filterByExpr( ) function; edgeR), as shown by limma's user guide. In other cases (‘most stringently filtered’ data, ‘SHOGUN TCGA data’, ‘Kraken TCGA data matched to SHOGUN TCGA data’ and both plasma microbiome data sets), quantile normalization was used because downstream SNM correction was not compatible with stringently filtered TMM normalized, feature-dropped data, as these data sets already had significantly reduced or low feature counts. With the exception of ‘most stringently filtered’ data, all quantile-normalized data sets were compared only to other quantile-normalized data sets. Principal components were calculated before and after SNM correction of the Voom-adjusted data, and principal variance components analysis (PVCA) quantified these changes between raw count data, Voom-adjusted data, and Voom-SNM normalized data. The mathematical basis for PVCA is well described by the NIEHS and the one tunable parameter was set to 80% based on their recommendation of 60-90%.

### Using SourceTracker2 as a Validation Analysis to Address Contamination Concerns

Shotgun sequencing data from the NIH's HMP2 Project, which swabbed eight body sites among 217 total samples, were downloaded and run against the same TCGA Kraken microbial-detection pipeline as described above, including against the same microbial database (n=59,974 bacterial, archaeal, and viral metagenomes) for taxonomy assignments. HMP2 data were summarized at the genus level, per the TCGA cancer microbiome data, and then were used to train a Bayesian source tracking model (SourceTracker2). Using SourceTracker parlance, these HMP2 samples served as ‘sources’ while the Voom-SNM-normalized samples acted as ‘sinks’, and the SourceTracker algorithm was used to calculate the proportion of each source attributable to each sink. In lay terms, the proportion of body sites from HMP2 data attributable to each Voom-SNM-normalized cancer microbiome sample using the Bayesian model was estimated. After (i) intersecting the genera in the cancer microbiome data set with themes in HMP2, (ii) converting the log₂(cpm)-normalized values to scaled relative abundances (scaled by 106 to give approximately 1 million total reads, as HMP2 data has 917,450 reads), and (iii) converting the data to BIOM table format, the model was applied to solid-tissue normal samples from the TCGA COAD cohort (n=70) and on primary tumor SKCM samples (n=122). SKCM primary tumor samples were chosen instead of solid-tissue normal samples as the best proxy of skin flora, as only one adjacent solid-tissue normal sample for SKCM was available. SourceTracker2 default settings were used for both runs. The outputs were calculated in terms of mean fractional contributions of each source to each sink; averages and standard errors of these values were subsequently calculated. Statistical differences between the fecal contributions to COAD and SKCM samples (FIG. 12b) were calculated using a one-sided Mann-Whitney U-test. The above protocol was repeated for the four decontaminated data sets to generate FIGS. 13c-f.

### TCGA ML Benchmarking and Generalizability

As a benchmarking and generalizability assessment, TCGA was split into two stratified data halves (across sequencing center, sample type, and disease type) of raw Kraken-derived, genus-level microbial count data (split #1: n=8,814; split #2, n=8,811), ran them both separately through the Voom-SNM protocol, built separate ML models on each normalized half, and then tested these tuned ML models on each other's normalized data. These model performances were then compared against a third ML model that was built on the full Voom-SNM-normalized data set (n=17,635 samples) and used 50-50% training and testing splits. Final performance was compared across all three approaches using their respective 50% holdout test set AUROC and AUPR. For additional internal validation, models were built to predict one cancer type versus all others using just (i) RNA samples or (ii) DNA samples, as well as on (iii) samples from one sequencing center that only did RNA-seq (UNC) or (iv) DNA-seq (HMS) (FIG. 10)

### TCGA Decontamination Analyses

Broadly speaking, there are two classes of possible contamination that affect next-generation sequencing data: external contamination (for example, reagents, investigators' or subjects' bodies, environmental contributions) and internal contamination (that is, cross-contamination between samples during processing or sequencing). In at least one embodiment, an overall decontamination approach attempts to (i) simulate contamination to estimate its contribution to predictive performance and/or model unreliability, (ii) mitigate external contamination as much as possible, and (iii) measure the degree of internal contamination using sensible positive and negative controls. External contaminants were identified and removed using sample analyte concentrations for all TCGA samples (n=17,625), as recently described and by using a blacklist of microorganisms identified from reagents in sequencing kits similar to those used in TCGA. Internal contaminants are particularly difficult to identify without having access to the primary samples or knowing which other samples (especially non-cancer samples) were run at the same time. As such, the only internal contaminants that were identified and removed as clear cross-contaminants were four reads assigned to the Ebolavirus genus (two reads from one TCGA-LGG sample at The Broad Institute and two reads from one TCGA-HNSC sample at HMS), almost certainly from concurrent studies on the 2014 West Africa outbreak at these same sequencing centers during the TCGA study collection period (2006-2016), and four reads assigned to the Marburgvirus genus (from two TCGA-OV samples at The Broad Institute), also probably of similar origin or as false positives (that is, Ebolavirus and Marburgvirus are both of the Filoviridae family). Doing so is in line with previously published work that removes microbial assignments that cannot be related to the biology at hand. It is further unlikely that such cross-contaminants, especially of extremely low abundance, would drive uniform discriminatory signals between and within types of cancer collected over many centers and years. For other possible cross-contaminants, estimates of their contribution using Bayesian analyses (described above) of ecologically expected communities rather than their identification and removal.

First, five pseudo-contaminants were spiked into the raw data set (FIG. 13a, top right) to track them through decontamination, SNM, and ML. This included the following: (1) 1,000 reads across all samples from HMS; (2) 1,000 reads across all samples from HMS, Baylor College of Medicine, Washington University School of Medicine, and Canada's Michael Smith Genome Sciences Centre; (3) 1,000 reads across all samples from all sequencing centers; (4) 106 reads spiked across 100 randomly selected samples from HMS; and (5) 106 reads spiked across 1,000 randomly selected samples from all sequencing centers. The mean raw read count across all samples and taxa was 1,481.20, so pseudo-contaminants containing 1,000 reads can be considered ‘low-level’ background while those with 106 reads are considered ‘high-abundance’ spikes. If pseudo-contaminants are present in downstream ML models after training, three interpretations are available: evaluate the percent predictive contribution of the pseudo-contaminants via feature importance scores and decide whether it is negligible or not; eliminate any ranked model features below the pseudo-contaminant; or, most conservatively, flag the entire model as being unreliable.

As TCGA did not include any negative blank reagent tubes during sample processing, techniques described herein attempted to pair a microbial blacklist at the genus level that used similar reagents and/or library preparation kits. TCGA SOPs mainly used QIAGEN products (Qiagen, Valencia, CA) for extracting DNA and RNA in tissues (DNA/RNA AllPrep kit) and DNA in blood (QiaAmp Blood Midi Kit). Salter and colleagues described such a list (n=94 genera) for DNA extraction kits in metagenomic experiments, including from QiaAmp kits that used the same silica membrane-based DNA purification as those used in TCGA blood extractions, obtained across four years of ‘negative blank’ sequencing and three high-throughput sequencing centers. Additional putative external contamination was identified on the basis that sequences from contaminants generally have frequencies that are inversely correlated with sample analyte concentration. A robust statistical framework recently validated this principle16, providing the opportunity to exploit sample DNA or RNA concentrations recorded by TCGA as a means to identify putative contaminants. The two main assumptions of this framework are (i) the contaminants are added in uniform amounts across samples; and (ii) the amount of contaminant DNA or RNA is small relative to the true sample DNA or RNA (microbial or host). Filtering was then conducted using the associated decontam R package (s://github.com/benjjneb/decontam) using the recommended hyperparameter threshold (P*=0.1) and a more stringent approach (P*=0.5). Note, P*=0.5 means that taxonomies are classified as ‘contaminant’ or ‘not’ if the contaminant model or non-contaminant model fit the distribution better. As it was found that sequencing center contributed substantial variation to the raw count data, the data was processed in batches corresponding to them, whereby a taxon identified as a contaminant at any center was subsequently discarded for all centers (that is, batch.combine=“minimum” in decontam). Putative lists of contaminants (P*=0.1: n=283 genera; P*=0.5: n=1,818 genera) were then combined/intersected with the microbial blacklist (n=94 genera) and subtracted from the full data set. Manual literature inspection of the smaller combined contaminant list (n=377) re-allowed 89 genera that were potentially pathogens or commensals. This resulted in three new data sets: ‘likely contaminants removed’, ‘all putative contaminants removed’, and ‘most stringent filtering’. As a further conservative measure, TCGA sample barcodes (for example, TCGA-02-0001-01C-01D-0182-01 were taken; as shown on NCI's documentation s://docs.gdc.cancer.gov/Encyclopedia/pages/TCGA_Barcode/) and extracted all sequencing plate—sequencing center combinations, as named by the barcode's last two sets of integers (that is, plate 0182 from center 01, or 0182-01, in this example). As decontam calculates the equivalent of a linear regression between taxon read fractions and analyte concentrations for all samples in a batch to determine whether a given taxon is classified as a contaminant, more than 10 samples per plate-center combination were required to qualify as a batch, giving 351 total plate-center batches. P*=0.1 was used (default value), and, as before, if a taxon was identified as a contaminant in any one of the 351 batches (batch.combine=“minimum”), it was removed from the data set (n=421 taxa removed). After intersecting with the microbial blacklist, a total of 497 genera were removed. This provided the fourth decontaminated data set, and all of them were then processed through the same SNM and ML pipelines described above.

### Comparing ML Performances Between BWA, SHOGUN, and Kraken Data

BWA filtering occurred against the same database used to generate the Kraken-based assignments (n=59,974 microbial genomes (bacteria, archaea and viruses)). Then, filtered BWA microbial count data were batch corrected in the same way as the Kraken data via Voom-SNM, except that DNA and RNA data were normalized separately owing to confounding between experimental strategy and sequencing center of the reduced sample count. Samples from the raw Kraken-derived data were then matched against samples processed by BWA and normalized in the same way as the BWA data. This resulted in a total of four normalized data sets: DNA BWA data, RNA BWA data, DNA Kraken-subsetted data, and RNA Kraken-subsetted data. All four normalized data sets were then inputted for ML and their performances were compared to each other (FIGS. 11a-h).

The ‘Web of Life’ database used for SHOGUN taxonomy assignments did not contain viruses, and SHOGUN processed a subset of all TCGA samples evaluated by Kraken (13,517 versus 17,625 samples). Thus, to make a fair comparison between their downstream ML performances, raw Kraken count data were subsetted to remove all identified viruses and to match the same samples processed by SHOGUN. Both data sets were then identically normalized by Voom (using quantile normalization) and SNM algorithms (using the same biological and technical variables as in the main TCGA analysis described above) before being fed into the ML pipelines for discrimination between and within types of cancer.

### Complementary Diagnostic Analyses

When evaluating the applicability of blood mbDNA to low-grade cancers, all patients with stage Ia-c and IIa-c classified tumors were grouped together and all others were discarded. For comparisons against the Guardant360 and FoundationOne Liquid ctDNA assays, all TCGA patients with at least one genomic alteration evaluated on their coding gene panels were filtered out; this included whether mutations were considered to be passengers or drivers. Remaining patients were used for ML analyses as described above.

### TCGA Simulations to Estimate Required Sample Sizes for Validation Study

To estimate the number of required samples from prostate, lung, and skin cancer (melanoma) for discrimination, empirical simulations on TCGA blood samples were performed at two different sequencing centers (Broad, HMS) that were all sequenced on one type of platform (Illumina HiSeq). First, Kraken-derived microbial count data was used and then repeated the simulations with SHOGUN-derived microbial count data. This most closely mimicked the expected real-world experimental conditions of the validation study.

First, all TCGA PRAD, LUAD, LUSC, and SKCM blood samples at Broad and HMS that were sequenced on Illumina HiSeq machines were subsetted from the raw Kraken data of microbial counts (Broad: n=99; HMS: n=288). Lung cancer samples used were of mixed origin, so LUAD and LUSC blood samples were combined into a single non-small-cell lung cancer (NSCLC) umbrella disease type; however, this applied only to Broad samples, as all blood-derived lung cancer samples at HMS were LUAD in origin. This left a breakdown of samples as follows: HMS: 66 LUAD, 104 PRAD, 118 SKCM; Broad: 42 NSCLC (24 LUAD, 18 LUSC), 17 PRAD, 40 SKCM. Then, each raw count data set for HMS and Broad was independently normalized through Voom (using quantile normalization) and SNM algorithms, using disease type as the biological variable of interest and tissue source site as the technical variable, as all other technical factors were precluded by picking a single sequencing center, data type, and platform.

The simulations were performed as follows on the normalized data sets: (1) random stratified sampling picked equal numbers of samples from the three classes; (2) one sample of the three-class subsample was left out; (3) an ML model was built on all the remaining samples in the subsample and applied on the left-out sample to make a prediction with a certain probability; (4) steps 2-3 were repeated until all samples had been iterated through; (5) using the list of observed classes and list of predicted classes along with their probabilities, multi-class performance metrics were estimated; (6) another stratified random sample was selected of the same sample size and steps 2-5 were repeated nine more times (a total of ten times) to estimate standard errors of the multi-class performance metrics; (7) steps 1-6 were repeated for individual class sample sizes of 5-40 with a step size of five samples. In cases where the stratified sampling size was larger than the number of samples in a class, all samples in that class were used. Collectively, this provided an estimate of the number of samples required to perform multi-cancer discrimination well (FIG. 16a). The empirical performance estimates (mean AUROC, mean AUPR) suggest that having at least 15 samples per cancer class should be sufficient. Note that it was not possible to estimate an ideal sample size for healthy controls because TCGA did not include them.

### Clinical Cohort Selection and IRB Protocols Numbers

Biobanked, frozen plasma samples from 169 patients were analyzed as part of this study, all from UC San Diego. All studies were approved by the Institutional Review Board (IRB) at UC San Diego, and under their respective IRB-approved protocols, patients provided written informed consent for sample donation and study. All prostate cancer plasma samples (n=59) came under IRB protocol 131550. All lung cancer and melanoma plasma samples came under IRB protocol 150348. All cancer- and HIV-free healthy control subjects (n=69) came under the following IRB protocol numbers: 130296, 091054, 172092, 151057, and 182064.

### Plasma-Derived, Cell-Free Microbial DNA Sample Processing, and Sequencing

Total circulating DNA was extracted from a volume of 250 μl plasma from each sample using the QIAamp Circulating Nucleic Acid Kit (QIAGEN) according to the manufacturer's instructions, and purified with AMPure XP SPRI paramagnetic beads (Beckman Coulter). Sequencing libraries were prepared from purified cfDNA using the KAPA HyperPlus Kit (Kapa Biosystems) with standard Illumina indexed adapters (IDT) as described. Sample libraries were characterized using the Agilent 4200 TapeStation System (High Sensitivity DNA Kit) and quantified by qPCR using the NEBNext Library Quant Kit for Illumina (New England Biolabs). Paired-end 2×150-bp sequencing (S4 flow cell) was performed on a NovaSeq 6000 instrument (Illumina), and samples were pooled across all four lanes during sequencing.

### Bioinformatic Processing for Plasma Microbiome Samples

A total of 21,600,141,264 reads were generated on the single NovaSeq 6000 sequencing run across all samples. Of these, 19,046,611,360 reads were assigned to human samples (that is, negative and positive controls removed), and 2.186% of the total reads were classified as non-human. Raw sequencing data were demultiplexed and adaptor-trimmed using Atropos. Additional quality filtering was done using Trimmomatic with the following settings—(ILLUMINACLIP:TruSeq3-PE-2.fa:2:30:7, MINLEN:50, TRAILING:20, AVGQUAL:20, SLIDINGWINDOW:20:20). An additional adaptor sequence consisting of a string of only G was added to the standard TruSeq3 adapters to remove trailing G stretches from the 5′ ends of reads. Read pairs were discarded if either mate mapped to the human genome (major-allele-SNP reference from 1000 Genomes Project) using Bowtie2 with the fast-local parameter set. Paired-end reads were then merged using FLASH with the following parameters—(minimum overlap: 20, maximum overlap: 150, mismatch ratio: 0.01).

The filtered, merged reads were then processed either by Kraken, using the same workflow and database (n=59,974 microbial genomes) detailed above, or with SHOGUN as detailed here. Samples were processed on individual plasma microbiome samples (that is, on a per-sample-per-lane basis, as samples were pooled across all four sequencing flow cells during the run). After per-sample-per-lane taxonomy assignment by Kraken or SHOGUN, microbial counts across lanes were aggregated for each sample after hierarchical clustering procedures showed consistent grouping by sample IDs rather than by flow cell lane. For SHOGUN-derived data, both successfully merged and unmerged reads were used as input for the SHOGUN align function, using Bowtie2 to map reads against the WoL database to generate taxonomy profiles, which were then collapsed to the genus level using QIIME 2. The taxonomy profiles of each sample were then filtered to remove all taxa whose relative abundance was less than 0.01%.

### Plasma Microbiome Technical Validation and Data Decontamination

To evaluate the performance of the sequencing run and bioinformatic microbial-detection pipelines, spiked wells and experimental serial dilutions of Aliivibrio fischeri (genus: Aliivibrio) included on the sequencing plate were examined against other sample types for differential abundance and in isolation for log-fold changes in abundance across dilutions. These technical positive controls are plotted in FIGS. 16b-c for both Kraken and SHOGUN-derived taxonomy assignments.

Three kinds of negative blank controls were included on the sequencing plate: (1) DNA extraction blanks, which had reagents from the DNA extraction stage through sequencing; (2) DNA library preparation blanks, which had reagents from the library preparation stage through sequencing; and (3) empty control wells, which had water added to them and then reagents during library preparation and would contain splashed and/or aerosolized microbial nucleic acids. As in the TCGA analysis, decontam was again used to decontaminate the plasma microbial data, except that it had access to both negative blank controls and DNA concentrations for all samples (excluding empty control wells for the latter). As a conservative measure, P*=0.5 hyperparameter value was selected for decontam for both ‘prevalence’ (that is, blank-based) and ‘frequency’ (that is, concentration-based) modes of decontamination; this hyperparameter value is equivalent to the most stringent decontamination in TCGA that discarded>90% of the total data. For prevalence mode, P*=0.5 will flag any taxon that is more prevalent in negative controls than biological ones as a contaminant; for frequency mode, P*=0.5 will flag any taxon whose model (that is, a regression model) fits a contaminant distribution better than a non-contaminant distribution using read fractions and DNA concentrations. For Kraken count data, prevalence mode discarded 21 taxa and frequency mode discarded 1,261 taxa (out of 1,753 original assignments); for SHOGUN count data, prevalence mode discarded 57 taxa and frequency mode discarded 244 taxa (out of 1,181 original assignments). Decontaminated data for both Kraken and SHOGUN were fed into downstream normalization and ML pipelines.

### Plasma Microbiome Data Normalization, Permutation Testing, and ML

An attempt to predict age using raw microbial count data was performed using GBM ML models (architectures same as those described above for TCGA) and leave-one-out (LOO) iterative ML (FIG. 16g).

To confirm the importance of normalizing for age and gender in this cohort, a permutation analysis was performed with 100 iterations for each factor and then simultaneously for both factors (FIGS. 16h-j). In brief, the following four steps were performed: (1) randomly swap age and/or sex labels among all samples; (2) run Voom-SNM on the raw data, using disease type as the biological variable of interest and permuted age and/or sex as the technical factors; (3) perform an ML analysis to discriminate grouped cancer samples from healthy controls using 70%-30% training—testing splits with a fixed random number seed and internal fourfold cross validation to obtain a two-class performance estimate (AUROC, AUPR); (4) repeat steps 1-3 for a total of 100 times to create a null performance distribution. Next, using correct, fixed age and/or sex assignments, steps 2-3 were run a total of 100 times while randomly selecting the random number seed in step 3. Last, this performance distribution was directly compared to its null distribution for significance using a two-sided Mann-Whitney U-test. As all of these tests were extremely significant (all P≤1.5×10⁻¹³), age and sex were incorporated as technical factors in the Voom-SNM while holding disease type as the biological variable of interest. Note, all lung cancer samples were labelled with a consolidated disease type label during normalization regardless of pathological subtype, as done in the TCGA cancer simulations (described above). All negative blank and positive monoculture controls were removed before Voom-SNM.

ML on the Voom-SNM normalized plasma microbiome samples was done exactly as previously described for TCGA samples, except for the sampling schema, because the sample sizes were smaller by orders of magnitude. First, to estimate generalization of healthy versus grouped cancer discriminations, ‘bootstrapping’ with 70%-30% training—testing splits with fourfold cross-validation during training for 500 iterations. Sampling with replacement was allowed in that every training—testing split (that is, every iteration) was unique; however, in no case was a sample allowed to be both a training case and a testing case. Summary statistics on the resultant performance metrics from all 500 iterations estimated the AUROC and AUPR distributions and confidence intervals (Cis) (FIG. 7b, FIG. 17a). Second, pairwise and multi-class discriminations between and among healthy controls and individual types of cancer were done with LOO ML. In other words, one sample was iteratively left out, a model was iteratively trained on the remaining samples with fourfold cross-validation for hyperparameter tuning, and a prediction was iteratively made on the left-out sample with a probability given by the model. The final list of actual classes for all samples was compared to the list of predicted classes and their probabilities to estimate AUROC and AUPR metrics, as described previously using the PRROC R package. Multi-class performance was estimated by taking the mean of all one-versus-all-others comparisons, as reported by the multiClassSummary( ) function in the caret R package.

Iterative subsampling to evaluate the contribution of smaller samples sizes to the melanoma cohort performance (FIG. 16k) was done as follows: (1) perform random stratified sampling of a single cancer type and healthy controls of 16 samples each (32 total); (2) perform LOO iterative ML and evaluate performance on those 32 samples for healthy versus cancer discrimination; (3) repeat steps 1-2 100 times to estimate performance standard errors; (4) repeat steps 1-3 for each of the three types of cancer. The same process was also done for iterative subsampling of PC and LC cohorts to study the impact of decreased sample size on their discrimination. Note that the entire melanoma cohort was used during each stratified subsampling, as the goal was to compare its cohort size to the other sample sizes.

### Statistical Analyses

All statistical analyses were done using R version 3.4.3. The ggpubr package (s://github.com/kassambara/ggpubr) performed nonparametric statistical testing between groups and accounted for multiple hypothesis testing correction when necessary. Note that P values less than 2.2×10⁻¹⁶ cannot be accurately calculated by R, so P values less than this are listed as <2.2×10⁻¹⁶; it is not a range of P values. Measurements were taken from distinct samples and not by repeatedly measuring samples. Sample size estimates for the validation study came from empirical simulations with TCGA blood samples and relied on the GBM package, Caret package, and MLmetrics package (s://github.com/yanyachen/MLmetrics) for performing ML and multi-class performance estimation. All other multi-class performance estimates were calculated using the Caret and MLmetrics packages.

### Training and Inferencing Using Machine-Learning Models

Various techniques may be used to train and inference (e.g., predict) using machine-learning models, such as neural networks, according to at least one embodiment. In at least one embodiment, an untrained neural network is trained using a training dataset. Initial weight parameters of an untrained neural network may be set to an initial predetermined value, random numbers, etc. In at least one embodiment, a training framework is used to train a neural network using the training data set and update one or more weights of the neural network. The training framework may be any suitable training framework, such as a PyTorch framework, TensorFlow, Boost, Caffe, Microsoft Cognitive Toolkit/CNTK, MXNet, Chainer, Keras, Deeplearning4j, or other training framework. In at least one embodiment, training framework trains an untrained neural network and enables it to be trained using processing resources described herein to generate a trained neural network. In at least one embodiment, weights may be chosen randomly or by pre-training using a deep belief network. In at least one embodiment, training may be performed in either a supervised, partially supervised, or unsupervised manner.

In at least one embodiment, untrained neural network is trained using supervised learning, wherein training dataset includes an input (e.g., microbial profile) paired with a desired output for an input (e.g., tissue of origin prediction), or where training dataset includes input having a known output and an output of neural network is manually graded. In at least one embodiment, untrained neural network is trained in a supervised manner and processes inputs from training dataset and compares resulting outputs against a set of expected or desired outputs. In at least one embodiment, errors are then propagated back through untrained neural network. In at least one embodiment, training framework adjusts weights that control the untrained neural network during the training process. In at least one embodiment, training framework includes tools to monitor how well untrained neural network is converging towards a model, such as trained neural network, suitable to generating correct answers, such as in result, based on input data such as a new dataset. In at least one embodiment, training framework trains untrained neural network repeatedly while adjust weights to refine an output of untrained neural network using a loss function and adjustment algorithm, such as stochastic gradient descent. In at least one embodiment, training framework trains untrained neural network until untrained neural network achieves a desired accuracy. In at least one embodiment, trained neural network can then be deployed to implement any number of machine learning operations.

In at least one embodiment, untrained neural network is trained using unsupervised learning, wherein untrained neural network attempts to train itself using unlabeled data. In at least one embodiment, unsupervised learning training dataset will include input data without any associated output data or “ground truth” data. In at least one embodiment, untrained neural network can learn groupings within training dataset and can determine how individual inputs are related to untrained dataset. In at least one embodiment, unsupervised training can be used to generate a self-organizing map in trained neural network capable of performing operations useful in reducing dimensionality of new dataset. In at least one embodiment, unsupervised training can also be used to perform anomaly detection, which allows identification of data points in new dataset that deviate from normal patterns of new dataset.

In at least one embodiment, semi-supervised learning may be used, which is a technique in which in training dataset includes a mix of labeled and unlabeled data. In at least one embodiment, training framework may be used to perform incremental learning, such as through transferred learning techniques. In at least one embodiment, incremental learning enables trained neural network to adapt to new dataset without forgetting knowledge instilled within trained neural network during initial training.

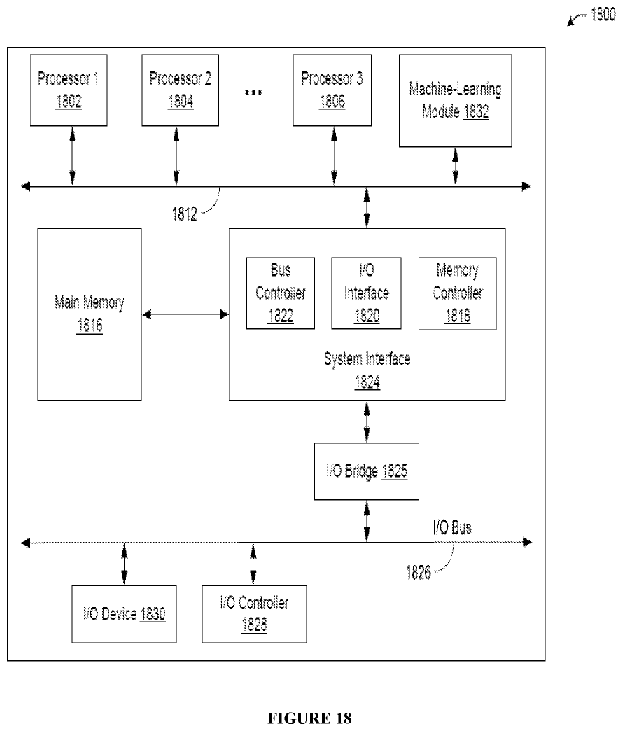

FIG. 18 is a block diagram illustrating an example of a computing device or computer system 1800 upon which any of one or more techniques (e.g., methods) may be performed, in accordance with one or more example embodiments of the present disclosure.

For example, the computing system 1800 of FIG. 18 may include one or more processors 1802-1806. Processors 1802-1806 may include one or more internal levels of cache (not shown) and a bus controller (e.g., bus controller 1822) or bus interface (e.g., I/O interface 1820) unit to direct interaction with the processor bus 1812.

Processor bus 1812, also known as the host bus or the front side bus, may be used to couple the processors 1802-1806 with the system interface 1824. System interface 1824 may be connected to the processor bus 1812 to interface other components of the system 1800 with the processor bus 1812. For example, system interface 1824 may include a memory controller 1818 for interfacing a main memory 1816 with the processor bus 1812. The main memory 1816 typically includes one or more memory cards and a control circuit (not shown). System interface 1824 may also include an input/output (I/O) interface 1820 to interface one or more I/O bridges 1825 or I/O devices 1830 with the processor bus 1812. One or more I/O controllers and/or I/O devices may be connected with the I/O bus 1826, such as I/O controller 1828 and I/O device 1830, as illustrated.

I/O device 1830 may also include an input device (not shown), such as an alphanumeric input device, including alphanumeric and other keys for communicating information and/or command selections to the processors 1802-1806. Another type of user input device includes cursor control, such as a mouse, a trackball, or cursor direction keys for communicating direction information and command selections to the processors 1802-1806 and for controlling cursor movement on the display device.

System 1800 may include a dynamic storage device, referred to as main memory 1816, or a random access memory (RAM) or other computer-readable devices coupled to the processor bus 1812 for storing information and instructions to be executed by the processors 1802-1806. Main memory 1816 also may be used for storing temporary variables or other intermediate information during execution of instructions by the processors 1802-1806. System 1800 may include read-only memory (ROM) and/or other static storage device coupled to the processor bus 1812 for storing static information and instructions for the processors 1802-1806. The system outlined in FIG. 18 is but one possible example of a computer system that may employ or be configured in accordance with aspects of the present disclosure.

According to one embodiment, the above techniques may be performed by computer system 1800 in response to processor 1804 executing one or more sequences of to one or more instructions contained in main memory 1816. These instructions may be read into main memory 1816 from another machine-readable medium, such as a storage device. Execution of the sequences of instructions contained in main memory 1816 may cause processors 1802-1806 to perform the process steps described herein. In alternative embodiments, circuitry may be used in place of or in combination with the software instructions. Thus, embodiments of the present disclosure may include both hardware and software components.

According to one embodiment, the processors 1802-1806 may include tensor processing units (TPUs) and/or other artificial intelligence accelerator application-specific integrated circuits (ASICs) that may allow for neural networking and other machine learning techniques. In at least one embodiment, machine-learning module 1832 refers to software and/or hardware that performs machine-learning techniques described herein, which may include training and/or inferencing stages. For example, machine-learning module 1832 may be trained to discriminate between different types and/or stages of metastatic cancer.

Various embodiments may be implemented fully or partially in software and/or firmware. This software and/or firmware may take the form of instructions contained in or on a non-transitory computer-readable storage medium. Those instructions may then be read and executed by one or more processors to enable the performance of the operations described herein. The instructions may be in any suitable form, such as, but not limited to, source code, compiled code, interpreted code, executable code, static code, dynamic code, and the like. Such a computer-readable medium may include any tangible non-transitory medium for storing information in a form readable by one or more computers, such as but not limited to read-only memory (ROM); random access memory (RAM); magnetic disk storage media; optical storage media; a flash memory, etc.

A machine-readable medium includes any mechanism for storing or transmitting information in a form (e.g., software, processing application) readable by a machine (e.g., a computer). Such media may take the form of, but is not limited to, non-volatile media and volatile media and may include removable data storage media, non-removable data storage media, and/or external storage devices made available via a wired to or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, solid state devices (SSDs), and the like. The one or more memory devices (not shown) may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in main memory 1816, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

The following references are hereby incorporated by reference:
Bullman, S. et al. Analysis of Fusobacterium persistence and antibiotic response in colorectal cancer. Science 358, 1443-1448 (2017).
Dejea, C. M. et al. Patients with familial adenomatous polyposis harbor colonic biofilms containing tumorigenic bacteria. Science 359, 592-597 (2018).
Geller, L. T. et al. Potential role of intratumor bacteria in mediating tumor resistance to the chemotherapeutic drug gemcitabine. Science 357, 1156-1160 (2017).
Gopalakrishnan, V. et al. Gut microbiome modulates response to anti-PD-1 immunotherapy in melanoma patients. Science 359, 97-103 (2018).
Jin, C. et al. Commensal microbiota promote lung cancer development via to γδ T cells. Cell 176, 998-1013.e16 (2019).
Ma, C. et al. Gut microbiome-mediated bile acid metabolism regulates liver cancer via NKT cells. Science 360, eaan5931 (2018).
Matson, V. et al. The commensal microbiome is associated with anti-PD-1 efficacy in metastatic melanoma patients. Science 359, 104-108 (2018).
Meisel, M. et al. Microbial signals drive pre-leukaemic myeloproliferation in a Tet2-deficient host. Nature 557, 580-584 (2018).
Routy, B. et al. Gut microbiome influences efficacy of PD-1-based immunotherapy against epithelial tumors. Science 359, 91-97 (2018).
Ye, H. et al. Subversion of systemic glucose metabolism as a mechanism to support the growth of leukemia cells. Cancer Cell 34, 659-673.e6 (2018).
The Cancer Genome Atlas Research Network et al. The Cancer Genome Atlas Pan-Cancer analysis project. Nat. Genet. 45, 1113-1120 (2013).
Hanahan, D. & Weinberg, R. A. The hallmarks of cancer. Cell 100, 57-70 (2000).
Hanahan, D. & Weinberg, R. A. Hallmarks of cancer: the next generation. Cell 144, 646-674 (2011).
Salter, S. J. et al. Reagent and laboratory contamination can critically impact sequence-based microbiome analyses. BMC Biol. 12, 87 (2014).
Glassing, A., Dowd, S. E., Galandiuk, S., Davis, B. & Chiodini, R. J. Inherent bacterial DNA contamination of extraction and sequencing reagents may affect interpretation of microbiota in low bacterial biomass samples. Gut Pathog. 8, 24 (2016).
Davis, N. M., Proctor, D. M., Holmes, S. P., Reiman, D. A. & Callahan, B. J. Simple statistical identification and removal of contaminant sequences in marker-gene and metagenomics data. Microbiome 6, 226 (2018).
Robinson, K. M., Crabtree, J., Mattick, J. S. A., Anderson, K. E. & Dunning Hotopp, J. C. Distinguishing potential bacteria-tumor associations from contamination in a secondary data analysis of public cancer genome sequence data. Microbiome 5, 9 (2017).
Eisenhofer, R. et al. Contamination in low microbial biomass microbiome studies: issues and recommendations. Trends Microbiol. 27, 105-117 (2019).
The Cancer Genome Atlas Research Network. Comprehensive molecular characterization of gastric adenocarcinoma. Nature 513, 202-209 (2014).
The Cancer Genome Atlas Research Network. Integrated genomic and molecular characterization of cervical cancer. Nature 543, 378-384 (2017).
Tang, K.-W., Alaei-Mahabadi, B., Samuelsson, T., Lindh, M. & Larsson, E. The landscape of viral expression and host gene fusion and adaptation in human cancer. Nat. Commun. 4, 2513 (2013).
Minich, J. J. et al. KatharoSeq enables high-throughput microbiome analysis from low biomass samples. mSystems 3, e00218-17 (2018).
Wood, D. E. & Salzberg, S. L. Kraken: ultrafast metagenomic sequence classification using exact alignments. Genome Biol. 15, R46 (2014).
Zhang, H. et al. Integrated proteogenomic characterization of human high-grade serous ovarian cancer. Cell 166, 755-765 (2016).
Choi, J.-H., Hong, S.-E. & Woo, H. G. Pan-cancer analysis of systematic batch effects on somatic sequence variations. BMC Bioinformatics 18, 211 (2017).
Lauss, M. et al. Monitoring of technical variation in quantitative high-throughput datasets. Cancer Inform. 12, 193-201 (2013).
Law, C. W., Chen, Y., Shi, W. & Smyth, G. K. voom: precision weights unlock linear model analysis tools for RNA-seq read counts. Genome Biol. 15, R29 (2014).
Mecham, B. H., Nelson, P. S. & Storey, J. D. Supervised normalization of microarrays. Bioinformatics 26, 1308-1315 (2010).
Boedigheimer, M. J. et al. Sources of variation in baseline gene expression levels from toxicogenomics study control animals across multiple laboratories. BMC Genomics 9, 285 (2008).
Scherer, A. Batch Effects and Noise in Microarray Experiments: Sources and Solutions (Wiley, 2009).
Hillmann, B. et al. Evaluating the information content of shallow shotgun metagenomics. mSystems 3, e00069-18 (2018).
Knights, D. et al. Bayesian community-wide culture-independent microbial source tracking. Nat. Methods 8, 761-763 (2011).
Integrative HMP (iHMP) Research Network Consortium. The Integrative Human Microbiome Project: dynamic analysis of microbiome-host omics profiles during periods of human health and disease. Cell Host Microbe 16, 276-289 (2014).
Yamamura, K. et al. Human microbiome Fusobacterium nucleatum in esophageal cancer tissue is associated with prognosis. Clin. Cancer Res. 22, 5574-5581 (2016).
Hsieh, Y.-Y. et al. Increased abundance of Clostridium and Fusobacterium in gastric microbiota of patients with gastric cancer in Taiwan. Sci. Rep. 8, 158 (2018).
Kostic, A. D. et al. PathSeq: software to identify or discover microbes by deep sequencing of human tissue. Nat. Biotechnol. 29, 393-396 (2011).
Svircev, Z. et al. Molecular aspects of microcystin-induced hepatotoxicity and hepatocarcinogenesis. J. Environ. Sci. Health C Environ. Carcinog. Ecotoxicol. Rev. 28, 39-59 (2010).
Jervis-Bardy, J. et al. Deriving accurate microbiota profiles from human samples with low bacterial content through post-sequencing processing of Illumina MiSeq data. Microbiome 3, 19 (2015).
Kwong, T. N. Y. et al. Association between bacteremia from specific microbes and subsequent diagnosis of colorectal cancer. Gastroenterology 155, 383-390.e8 (2018).
Blauwkamp, T. A. et al. Analytical and clinical validation of a microbial cell-free DNA sequencing test for infectious disease. Nat. Microbiol. 4, 663-674 (2019).
Hong, D. K. et al. Liquid biopsy for infectious diseases: sequencing of cell-free plasma to detect pathogen DNA in patients with invasive fungal disease. Diagn. Microbiol. Infect. Dis. 92, 210-213 (2018).
Burnham, P. et al. Urinary cell-free DNA is a versatile analyte for monitoring infections of the urinary tract. Nat. Commun. 9, 2412 (2018).
De Vlaminck, I. et al. Temporal response of the human virome to immunosuppression and antiviral therapy. Cell 155, 1178-1187 (2013).
Huang, Y.-F. et al. Analysis of microbial sequences in plasma cell-free DNA for early-onset breast cancer patients and healthy females. BMC Med. Genomics 11 (Suppl. 1), 16 (2018).
Bettegowda, C. et al. Detection of circulating tumor DNA in early- and late-stage human malignancies. Sci. Transl. Med. 6, 224ra24 (2014).
Clark, T. A. et al. Analytical validation of a hybrid capture-based next-generation sequencing clinical assay for genomic profiling of cell-free circulating tumor DNA. J. Mol. Diagn. 20, 686-702 (2018).
Sanders, J. G. et al. Optimizing sequencing protocols for leaderboard metagenomics by combining long and short reads. Genome Biol. 20, 226 (2019).
Huang S. et al. Human skin, oral, and gut microbiomes predict chronological age. mSystems 5, e00630-19 (2020).
Zhu, Q. et al. Phylogenomics of 10,575 genomes reveals evolutionary proximity between domains Bacteria and Archaea. Nat. Commun. 10, 5477 (2019).
Chiu, K.-P. & Yu, A. L. Application of cell-free DNA sequencing in characterization of bloodborne microbes and the study of microbe-disease interactions. PeerJ 7, e7426 (2019).
Lau, J. W. et al. The Cancer Genomics Cloud: collaborative, reproducible, and democratized—a new paradigm in large-scale computational research. Cancer Res. 77, e3-e6 (2017).
Hoadley, K. A. et al. Cell-of-origin patterns dominate the molecular classification of 10,000 tumors from 33 types of cancer. Cell 173, 291-304.e6 (2018).
Reynolds, S. M. et al. The ISB Cancer Genomics Cloud: a flexible cloud-based platform for cancer genomics research. Cancer Res. 77, e7-e10 (2017).
Ellrott, K. et al. Scalable open science approach for mutation calling of tumor exomes using multiple genomic pipelines. Cell Syst. 6, 271-281.e7 (2018).
The Cancer Genome Atlas Network. Comprehensive molecular portraits of human breast tumors. Nature 490, 61-70 (2012).
Cerami, E. et al. The cBio cancer genomics portal: an open platform for exploring multidimensional cancer genomics data. Cancer Discov. 2, 401-404 (2012).
Gao, J. et al. Integrative analysis of complex cancer genomics and clinical profiles using the cBioPortal. Sci. Signal. 6, pl1 (2013).
Land, M. L. et al. Quality scores for 32,000 genomes. Stand. Genomic Sci. 9, 20 (2014).
Li, H. & Durbin, R. Fast and accurate short read alignment with Burrows-to Wheeler transform. Bioinformatics 25, 1754-1760 (2009).
Greathouse, K. L. et al. Interaction between the microbiome and TP53 in human lung cancer. Genome Biol. 19, 123 (2018).
Shanmughapriya, S. et al. Viral and bacterial aetiologies of epithelial ovarian cancer. Eur. J. Clin. Microbiol. Infect. Dis. 31, 2311-2317 (2012).
Banerjee, S. et al. The ovarian cancer oncobiome. Oncotarget 8, 36225-36245 (2017).
Langmead, B. & Salzberg, S. L. Fast gapped-read alignment with Bowtie 2. Nat. Methods 9, 357-359 (2012).
Bolyen, E. et al. Reproducible, interactive, scalable and extensible microbiome data science using QIIME 2. Nat. Biotechnol. 37, 852-857 (2019).
Ritchie, M. E. et al. limma powers differential expression analyses for RNA-sequencing and microarray studies. Nucleic Acids Res. 43, e47 (2015).
Robinson, M. D., McCarthy, D. J. & Smyth, G. K. edgeR: a Bioconductor package for differential expression analysis of digital gene expression data. Bioinformatics 26, 139-140 (2010).
McDonald, D. et al. The Biological Observation Matrix (BIOM) format or: how I learned to stop worrying and love the ome-ome. 1, 2047-217X-1-7 (2012).
Friedman, J. H. Stochastic gradient boosting. Comput. Stat. Data Anal. 38, 367-378 (2002).
Friedman, J. H. Greedy function approximation: a gradient boosting machine. Ann. Stat. 29, 1189-1232 (2001).
Kuhn, M. Building predictive models in R using the caret package. J. Stat. Softw. 28, 1-26 (2008).
Grau, J., Grosse, I. & Keilwagen, J. PRROC: computing and visualizing to precision-recall and receiver operating characteristic curves in R. Bioinformatics 31, 2595-2597 (2015).
Gire, S. K. et al. Genomic surveillance elucidates Ebola virus origin and transmission during the 2014 outbreak. Science 345, 1369-1372 (2014).
Matranga, C. B. et al. Enhanced methods for unbiased deep sequencing of Lassa and Ebola RNA viruses from clinical and biological samples. Genome Biol. 15, 519 (2014).
Gonzalez, A. et al. Avoiding pandemic fears in the subway and conquering the platypus. mSystems 1, e00050-16 (2016).
Didion, J. P., Martin, M. & Collins, F. S. Atropos: specific, sensitive, and speedy trimming of sequencing reads. PeerJ 5, e3720 (2017).
Bolger, A. M., Lohse, M. & Usadel, B. Trimmomatic: a flexible trimmer for Illumina sequence data. Bioinformatics 30, 2114-2120 (2014).
The 1000 Genomes Project Consortium. A global reference for human genetic variation. Nature 526, 68-74 (2015).
Magoc̆, T. & Salzberg, S. L. FLASH: fast length adjustment of short reads to improve genome assemblies. Bioinformatics 27, 2957-2963 (2011).
Gonzalez, A. et al. Qiita: rapid, web-enabled microbiome meta-analysis. Nat. Methods 15, 796-798 (2018).

1. A method for determining a presence or lack thereof metastatic cancer of a subject, comprising:
(a) detecting a microbial presence in a biological sample of a subject with cancer;
(b) removing contaminated microbial features from the microbial presence, thereby producing a decontaminated microbial presence;
(c) comparing the decontaminated microbial presence to a microbial presence of one or more biological samples from one or more subjects with cancer, thereby generating a microbial-cancer comparison dataset; and
(d) determining the presence or lack thereof metastatic cancer of the subject from the microbial cancer comparison dataset.

2. The method of claim 1, wherein determining further comprising identifying a tissue of origin of the metastatic cancer.

3. The method of claim 1, wherein the one or more subjects with cancer of step (c) comprise primary tumors, metastatic tumors, or any combination thereof.

4. The method of claim 1, wherein the microbial presence further comprises a microbial abundance.

5. The method of claim 4, wherein the microbial presence or abundance comprises the following non-mammalian domains of life: bacteria, fungi, viruses, archaea, protozoa, bacteriophages, or any combination thereof.

6. The method of claim 4, wherein the microbial presence or abundance is measured by ecological shotgun sequencing, quantitative polymerase chain reaction, immunohistochemistry, in situ hybridization, flow cytometry, host whole genome sequencing, host transcriptomic sequencing, cancer whole genome sequencing, cancer transcriptomic sequencing, or any combination thereof.

7. The method of claim 4, wherein the microbial presence or abundance is measured by amplification of the following nucleic acid regions of microbial origin: V1, V2, V3, V4, V5, V6, V7, VS, V9 variable domain region of 16S rRNA, the internal transcribed spacer (ITS) region of the 18S rRNA, or any combination thereof.

8. The method of claim 4, wherein the microbial presence or abundance is detected by nucleic acid measurement that targets microbial DNA, RNA, or any combination thereof, wherein the nucleic acid measurement that targets microbial DNA, RNA, or any combination thereof, occurs simultaneously with a measurement of the subject's mammalian DNA, RNA, or any combination thereof.

9. The method of claim 1, wherein the metastatic cancer comprises: Acute Myeloid Leukemia, Adrenocortical Carcinoma, Bladder Urothelial Carcinoma, Brain Lower Grade Glioma, Breast Invasive Carcinoma, Cervical Squamous Cell Carcinoma and Endocervical Adenocarcinoma, Cholangiocarcinoma, Colon Adenocarcinoma, Lymphoid Neoplasm Diffuse Large B-cell Lymphoma, Esophageal Carcinoma, Glioblastoma Multiforme, Head and Neck Squamous Cell Carcinoma, Kidney Chromophobe, Kidney Renal Clear Cell Carcinoma, Kidney Renal Papillary Cell Carcinoma, Liver Hepatocellular Carcinoma, Lung Adenocarcinoma, Lung Squamous Cell Carcinoma, Mesothelioma, Ovarian Serous Cystadenocarcinoma, Pancreatic Adenocarcinoma, Pheochromocytoma and Paraganglioma, Prostate Adenocarcinoma, Rectum Adenocarcinoma, Sarcoma, Skin Cutaneous Melanoma, Stomach Adenocarcinoma, Testicular Germ Cell Tumors, Thyroid Carcinoma, Thymoma, Uterine Carcinosarcoma, Uterine Corpus Endometrial Carcinoma, Uveal Melanoma, or any combination thereof.

10. The method of claim 1, wherein the metastatic cancer comprises a cancer type, wherein the cancer type comprises: lung cancer, prostate cancer, melanoma cancer, breast cancer, thyroid cancer, or any combination thereof.

11. The method of claim 1, wherein the contaminated microbial features comprise taxonomic assignment of the microbial presence.

12. The method of claim 1, wherein step (b) improves an accuracy of determining the tissue of origin of the metastatic cancer.

13. The method of claim 1, wherein step (b) is omitted.

14. The method of claim 1, wherein the microbial-cancer comparison dataset further comprises mammalian features, wherein the mammalian features comprise: immunohistochemistry protein markers of tumor tissue, tumor tissue DNA, tumor tissue RNA, tumor tissue methylation patterns, cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, methylation patterns of circulating tumor cell derived RNA, or any combination thereof.

15. The method of claim 1, wherein the biological sample comprises a tissue sample, liquid biopsy, whole blood biopsy, or any combination thereof.

16. The method of claim 15, wherein the biological sample comprises one or more constituents of whole blood comprising: plasma, white blood cells, red blood cells, platelets, or any combination thereof.

17. A method of administering a treatment to treat metastatic cancer of a subject based on microbial presence, comprising:
(a) detecting a microbial presence m a biological sample from the subject with metastatic cancer;
(b) removing contaminated microbial features of the microbial presence, thereby producing a decontaminated microbial presence;
(c) generating an association between the decontaminated microbial presence and the metastatic cancer of the subject; and
(d) administering to the subject the treatment determined by the association between the decontaminated microbial presence and the metastatic cancer.

18. The method of claim 17, wherein the microbial presence further comprises a microbial abundance, wherein the microbial presence or abundance comprise the following non-mammalian domains of life: bacteria, fungi, viruses, archaea, protozoa, bacteriophages, or any combination thereof.

19. The method of claim 17, wherein the contaminated microbial features comprise taxonomic assignment of the microbial presence.

20. The method of claim 17, wherein step (b) is omitted.

21. The method of claim 17, wherein the biological sample comprises a tissue sample, liquid biopsy, whole blood biopsy, or any combination thereof.

22. The method of claim 21, wherein the biological sample comprises one or more constituents of whole blood comprising: plasma, white blood cells, red blood cells, platelets, or any combination thereof.

23. The method of claim 17, wherein the treatment is not metabolized or rendered inactive by the decontaminated microbial presence.

24. The method of claim 17, wherein the treatment comprises: a small molecule, a hormone therapy, a biologic, an engineered host-derived cell type or types, a probiotic, an engineered bacterium, a natural-but-selective virus, an engineered virus, a bacteriophage, or any combination thereof.

25. The method of claim 17, wherein the metastatic cancer comprises: Acute Myeloid Leukemia, Adrenocortical Carcinoma, Bladder Urothelial Carcinoma, Brain Lower Grade Glioma, Breast Invasive Carcinoma, Cervical Squamous Cell Carcinoma and Endocervical Adenocarcinoma, Cholangiocarcinoma, Colon Adenocarcinoma, Lymphoid Neoplasm Diffuse Large B-cell Lymphoma, Esophageal Carcinoma, Glioblastoma Multiforme, Head and Neck Squamous Cell Carcinoma, Kidney Chromophobe, Kidney Renal Clear Cell Carcinoma, Kidney Renal Papillary Cell Carcinoma, Liver Hepatocellular Carcinoma, Lung Adenocarcinoma, Lung Squamous Cell Carcinoma, Mesothelioma, Ovarian Serous Cystadenocarcinoma, Pancreatic Adenocarcinoma, Pheochromocytoma and Paraganglioma, Prostate Adenocarcinoma, Rectum Adenocarcinoma, Sarcoma, Skin Cutaneous Melanoma, Stomach Adenocarcinoma, Testicular Germ Cell Tumors, Thyroid Carcinoma, Thymoma, Uterine Carcinosarcoma, Uterine Corpus Endometrial Carcinoma, Uveal Melanoma, or any combination thereof.

26. The method of claim 17, wherein the treatment comprises an adjuvant given m combination with a primary treatment against the metastatic cancer to improve efficacy of the primary treatment.

27. The method of claim 26, wherein the adjuvant is an antibiotic or an anti-microbial.

28. The method of claim 17 wherein, the treatment is based on microbial constituents or antigens associated with the metastatic cancer or the metastatic cancer's environment.

29. The method of claim 28, wherein the treatment comprises an adoptive cell transfer to target microbial antigens, a cancer vaccine against microbial antigens, a monoclonal antibody against microbial antigens, an antibody-drug-conjugate designed to at least partially target microbial antigens, a multi-valent antibody, antibody fragment, antibody derivative thereof designed to at least partially target one or more microbial antigens, or any combination thereof.

30. The method of claim 17, wherein the treatment comprises an antibiotic targeted against a class of functionally or biologically similar microbes of the microbial presence.

31. The method of claim 28, wherein the treatment comprises two or more treatment types, wherein the two or more treatment types are combined such that at least one type of the two or more treatment types exploits the microbial presence or abundance associated with the metastatic cancer or the metastatic cancer environment to enhance therapeutic efficacy.

32. The method of claim 17, wherein the association between the decontaminated microbial presence and the metastatic cancer further comprises the origin, type, or any combination thereof the metastatic cancer.

33. A computer system configured to determine a presence or absence of metastatic cancer of a subject, comprising:
one or more processors; and
a non-transient computer readable storage medium including software, wherein the software comprises executable instructions that, as a result of execution, cause the one or more processors of the computer system to:
(a) obtain one or more nucleic acid molecules of a biological sample from the subject with cancer;
(b) separate microbial nucleic acids from non-microbial nucleic of the one or more nucleic acids of the biological sample;
(c) identify a microbial presence of the microbial nucleic acids;
(d) remove contaminated microbial features of the microbial presence, thereby producing a table of decontaminated microbial presence;
(e) input the table of decontaminated microbial presence into a machine-learning model; and
(f) receive from the machine-learning model, an output that indicates the presence or the absence of the metastatic cancer.

34. The computer system of claim 33, wherein the microbial presence further comprises a microbial abundance, wherein the microbial presence or abundance comprise the following non-mammalian domains of life: bacteria, fungi, viruses, archaea, protozoa, bacteriophages, or any combination thereof.

35. The computer system of claim 33, wherein the decontaminated microbial features comprise taxonomic assignment of the microbial presence.

36. The computer system of claim 33, wherein step (d) is omitted.

37. The computer system of claim 33, wherein microbial and non-microbial nucleic acids are separated by aligning the one or more nucleic acid molecules against a reference database of microbial and non-microbial genomes.

38. The computer system of claim 33, wherein the microbial and non-microbial nucleic acids are separated without aligning the one or more nucleic acid molecules against a reference genome database.

39. The computer system of claim 33, wherein the table of decontaminated microbial presence further comprise mammalian features, wherein the mammalian features comprise: immunohistochemistry protein markers of tumor tissue, tumor tissue DNA, tumor tissue RNA, tumor tissue methylation patterns, cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, methylation patterns of circulating tumor cell derived RNA, or any combination thereof.

40. The computer system of claim 33, wherein the metastatic cancer comprises: Acute Myeloid Leukemia, Adrenocortical Carcinoma, Bladder Urothelial Carcinoma, Brain Lower Grade Glioma, Breast Invasive Carcinoma, Cervical Squamous Cell Carcinoma and Endocervical Adenocarcinoma, Cholangiocarcinoma, Colon Adenocarcinoma, Lymphoid Neoplasm Diffuse Large B-cell Lymphoma, Esophageal Carcinoma, Glioblastoma Multiforme, Head and Neck Squamous Cell Carcinoma, Kidney Chromophobe, Kidney Renal Clear Cell Carcinoma, Kidney Renal Papillary Cell Carcinoma, Liver Hepatocellular Carcinoma, Lung Adenocarcinoma, Lung Squamous Cell Carcinoma, Mesothelioma, Ovarian Serous Cystadenocarcinoma, Pancreatic Adenocarcinoma, Pheochromocytoma and Paraganglioma, Prostate Adenocarcinoma, Rectum Adenocarcinoma, Sarcoma, Skin Cutaneous Melanoma, Stomach Adenocarcinoma, Testicular Germ Cell Tumors, Thyroid Carcinoma, Thymoma, Uterine Carcinosarcoma, Uterine Corpus Endometrial Carcinoma, Uveal Melanoma, or any combination thereof.

41. The computer system of claim 33, wherein the metastatic cancer comprises a cancer type, wherein the cancer type comprises: lung cancer, prostate cancer, melanoma cancer, breast cancer, thyroid cancer, or any combination thereof.

42. The computer system of claim 33, wherein the biological sample comprises a tissue sample, liquid biopsy, whole blood biopsy, or any combination thereof.

43. The computer system of claim 33, wherein the biological sample comprises constituents of whole blood comprising: plasma, white blood cells, red blood cells, platelets, or any combination thereof.

44. The computer system of claim 33, wherein the machine-learning model is trained to discriminate between non-metastatic and metastatic cancerous tissue or blood samples.

45. The computer system of claim 33, wherein the machine-learning model is trained to differentiate one or more cancer types.

46. The computer system of claim 45, wherein the one or more cancer types comprises: lung cancer, prostate cancer, melanoma cancer, breast cancer, thyroid cancer, or any combination thereof.

47. The computer system of claim 33, wherein the output further comprises an indication of type, tissue of origin, or any combination thereof the metastatic cancer.
